SEQUENCE LISTING (20 sequences)

SEQ ID NO: 1 - protein, 5 aa, synthetic construct
  REGION 1..5: note = Linker Sequence
RTVAA

SEQ ID NO: 2 - protein, 5 aa, synthetic construct
  REGION 1..5: note = Linker Sequence
GGGGS

SEQ ID NO: 3 - protein, 6 aa, synthetic construct
  REGION 1..6: note = Linker Sequence
GGSGGS

SEQ ID NO: 4 - protein, 9 aa, synthetic construct
  REGION 1..9: note = Linker Sequence
GGSGGSGGS

SEQ ID NO: 5 - protein, 10 aa, synthetic construct
  REGION 1..10: note = Linker Sequence
GGGGSGGGGS

SEQ ID NO: 6 - protein, 122 aa, synthetic construct [sequence omitted]

SEQ ID NO: 7 - protein, 107 aa, synthetic construct [sequence omitted]

SEQ ID NO: 8 - protein, 124 aa, synthetic construct
  REGION 1..124: note = VH Sequence
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSS

SEQ ID NO: 9 - protein, 111 aa, synthetic construct
  REGION 1..111: note = VL Sequence
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 10 - protein, 237 aa, synthetic construct
  REGION 1..237: note = Polypeptide A
EIVLTQSPATLSVTPGERATLSCSASSSVSYMHWYQKKPGQAPRLLIYDTSKLASGIPARFSGSGSGTDFTLTISSLEPEDFAVYYCFQGSVYPFTFGCGTKLEIKRTVAAEVQLVESGGGLVQPGGSLRLSCAASGFTFNTYAMNWVRKAPGKGLEWVGRIRSKYNNYATYYADSVDKGRFTISRDDSKNSLYLQMNSLKTEDTAVYYCVRHGNFGNSYVSWFAYWGQGTLVTVSS

SEQ ID NO: 11 - protein, 467 aa, synthetic construct
  REGION 1..467: note = Polypeptide B
EAVVTQEPSLTVSPGGTVTLTCRSSTGAVTTSNYANWVQDKPGQAPRGLIGGTNKRAPWTPARFSGSLLGGKAALTITGAQPEDEAGYYCALWYSNLWVFGGGTKLTVLRTVAAQVQLQESGPGLVKPSQTLSLTCTVSGGSISTSGMGVGWIRDHPGKCLEWIGHIWWDDDKRYNPALKSRVTISVDTSKNQFSLKLSSVTAADTAVYYCARMELWSYYFDYWGQGTTLTVSSASEPKSDKTHTCPPCPAPPVAGGPSVPLPPPKPKDTLMISRTPEVTCVVVDVSQEDPEVKFNWYVDGVGVHNAKTKPREEQFNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 227 aa, synthetic construct
  REGION 1..227: note = Polypeptide C
DKTHTCPPCPAPPVAGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 13 - protein, 106 aa, synthetic construct
  REGION 1..106: note = CD19 VL
EIVLTQSPATLSVTPGERATLSCSASSSVSYMHWYQQKPGQAPRLLIYDTSKLASGIPARFSGSGSGTDFTLTISSLEPEDFAVYYCFQGSVYPFTFGQGTKLEIK

SEQ ID NO: 14 - protein, 126 aa, synthetic construct
  REGION 1..126: note = CD19 VH
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTTLTVSSASEPKS

SEQ ID NO: 15 - protein, 109 aa, synthetic construct
  REGION 1..109: note = CD3 VL
EAVVTQEPSLTVSPGGTVTLTCRSSTGAVTTSNYANWVQQKPGQAPRGLIGGTNKRAPWTPARFSGSLLGGKAALTITGAQPEDEAEYYCALWYSNLWVFGGGTKLTVL

SEQ ID NO: 16 - protein, 126 aa, synthetic construct
  REGION 1..126: note = CD3 VH
EVQLVESGGGLVQPGGSLRLSCAASGFTFNTYAMNWVRQAPGKGLEWVGRIRSKYNNYATYYADSVDKGRFTISRDDSKNSLYLQMNSLKTEDTAVYYCVRHGNFGNSYVSWFAYWGQGTLVTVSS

SEQ ID NO: 17 - protein, 451 aa, synthetic construct
  REGION 1..451: note = Polypeptide D
QVQLVESGGGVVQPGRSLRLSCAASGPTFSDFGMNWVRKAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 18 - protein, 213 aa, synthetic construct
  REGION 1..213: note = Polypeptide E
DIQMTQSPSSLSASVGDRVTITCKGSQDINNYLAWYQDKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 107 aa, synthetic construct
  REGION 1..107: note = CD8 VL
DIKMTQSPSSLSASVGDRVTITCRTSRSISQYLAWYQEKPGKTNKLLIYSGSTLQSGIPSRFSGSGSGTDFTLTISSLQPEDFATYYCQQHNENPLTFGAGTKLEIK

SEQ ID NO: 20 - protein, 118 aa, synthetic construct
  REGION 1..118: note = CD8 VH
EVQLQQSGAEVKKPGASVKVSCKASGFNIKDTYIHWVRQAPGKGLEWMGRIDPANDNTLYASKFQGRVTITADTSTNTAYMELSSLRSEDTAVYYCGRGYGYYVFDHWGQGTTVTVSS

(19) United States
(12) Patent Application Publication
(10) Pub. No.: US 2026/0327347 A1
(43) Pub. Date: Oct. 22, 2026

(54) COVALENT MULTI-SPECIFIC ANTIBODIES

(71) Applicant: Chimagen Biosciences, Ltd., Chengdu (CN)

(72) Inventors: Zhenhao ZHOU, Chengdu (CN); Jie ZHANG, Chengdu (CN)

(21) Appl. No.: 19/733,788

(22) Filed: Jul. 7, 2026

Related U.S. Application Data
(63) Continuation of application No. 15/733,195, filed on Dec. 20, 2018.

(30) Foreign Application Priority Data
Dec. 22, 2017 (CN) 201711415979.9

(51) Int. Cl. C07K 16/46; A61K 39/00; C07K 16/28

(52) U.S. Cl. CPC C07K 16/468; C07K 16/2803; C07K 16/2809; A61K 2039/505; C07K 2317/31; C07K 2317/56; C07K 2317/73; C07K 2317/92; C07K 2317/94

(57) ABSTRACT

The present invention relates to novel covalent multi-specific antibodies with increased stability, and use thereof for therapy, such as for immunotherapy.

### CROSS REFERENCE TO RELATED APPLICATIONS

This application is a Continuation application of U.S. patent application Ser. No. 15/733,195, filed on Jun. 8, 2020, which is a 371 National Phase Application of PCT Application No. PCT/CN2018/122321 entitled “COVALENT MULTI-SPECIFIC ANTIBODIES”, filed on Dec. 20, 2018, which claims the benefit of, and priority to, Chinese Patent Application Serial No. 201711415979.9, entitled “COVALENT MULTI-SPECIFIC ANTIBODIES”, filed on Dec. 22, 2017, the entire disclosures of which are incorporated herein by reference in their entireties.

### INCORPORATION BY REFERENCE OF SEQUENCE LISTING

The instant application contains a Sequence Listing which has been submitted electronically in XML format and is hereby incorporated by reference in its entirety. Said XML copy, created on Jun. 18, 2026, is named JUN-001US3_SL.xml and is 28,341 bytes in size.

### FIELD OF THE INVENTION

The present invention relates to novel covalent multi-specific antibodies with increased stability, and use thereof for therapy, such as for immunotherapy.

### BACKGROUND OF THE INVENTION

Monoclonal antibodies (mAbs) have wide diagnostic and therapeutic potentials in clinical practices against cancer and other diseases. Monoclonal antibodies play a central role in cancer immunotherapy, either in naked forms, or as conjugates to cytotoxic agents, such as radioisotopes, drugs, toxins, or prodrug-converting enzymes. These approaches are under active evaluation, with different levels of developmental and clinical successes. Naked mAbs potentially may achieve clinical responses by inducing a cytotoxic effect upon binding to cell surface proteins that are over-expressed on cancer cells. Studies have shown that these therapeutic effects were accomplished by controlling tumor growth via programmed cell death (apoptosis), or by the induction of anti-tumor immune responses.

Because its unique features of specific targeting and mediating effector functions, antibody was explored as drug for targeting immunotherapy against diseases since the invention of monoclonal antibody technology by Cesar Milstein and Georges J. F. Kohler on 1975. There are currently more than 60 approved antibody-based biologic drugs with global annual sales of >$50 billion. The successful application of the current generation of antibody drugs has shaped the pharmaceutical industry and has been greatly improving public health. The development of optimal combinational therapies and innovative bi-specific antibodies, in addition to the development of antibody drugs against novel targets, are among the perspective future directions.

Therapeutic antibodies have been used in clinical applications for over twenty years. Currently, there are many anti-tumor antibody drugs in clinic, including Rituxan (1997), Herceptin (1998), Mylotarg (2000), Campath (2001), Zevalin (2002), Bexxer (2003), Avastin (2004), Erbitux (2004), Vectibix (2006), Arzerra (2009); Benlysta (2011); Yervoy (2011), Adcetris (2011), Perjeta (2012), Kadcyla (2013), Opdivo (2014), Keytruda (2014), Tecentriq (2016). These antibodies target mainly EGFR, Her2, CD20 or VEGF, and more recently PD1 or PD-L1.

Multi-functional antibodies are constructed based on traditional antibodies through sophisticated design and molecular engineering, which enable the antibodies to bind to more than one antigen. Practically one single molecule is capable of delivering therapeutic effects same as that from a combination of several conventional antibodies. However, advantages of multi-functional antibodies go beyond the simple additive effect. Simultaneous engagement of multiple targets of selection can deliver benefits superior than classic antibodies via novel and unique mechanisms. For example, Blinatumomab (CD3×CD19, Amgen) that targets CD3 and CD19 can efficiently engage T cells in the killing of CD19-expressing tumor cells via its CD3-recognizing Fv and showed superior efficacy over conventional antibodies in treating ALL (acute lymphoid leukemia) etc. Blinatumomab was approved to launch for ALL treatment by FDA in 2014.

A number of bi-specific antibody technology platforms have been developed, to name a few among them: BiTE Bi-specific T-cell Engaging (Micromet, acquired by Amgen in 2012), CrossMab (Roche), DVD-Ig (Abbvie), TandAb (Affimed), DART (Dual Antigen Re-Targeting, Macrogenics). These platforms adopt different methods for antibody construction; each has distinct merit yet bears its own disadvantage: BITE antibodies are less stable with the tendency for aggregation in spite of their high potency; the CrossMab platform engages complicated methods for antibody construction and requires customized modifications for individual parental antibodies involved; the proximal Fv of DVD-Ig antibodies can only bind to soluble antigens due to its incapability to interact with membrane proteins; the TandAb platform produces antibodies of which the two chains are only connected through the VH-VL interaction (through formation of a hydrophobic core at the interphase), while the antibodies have very high avidity and affinity in vitro, they quickly lose activity due to dissociation of the double-chain once they are in vivo and end up with a fairly short half-life. Besides, relatively high level of mismatch is a common problem for some bi-specific antibody platforms. This excludes the possibility of using the traditional classic procedures for antibody purification and poses obstacles to downstream development processes. Furthermore, in most cases construction of a bi-specific antibody impairs the bivalent binding capability of the antibody for individual antigens, thus lowers its selectivity and avidity for the antigens to various extent.

Bispecific antibodies have been produced by chemical cross-linking, by hybrid-hybridomas or transfectomas, or by disulfide exchange at the hinge of two different Fab′. The first method yields heterogeneous and ill-defined products. The second method requires extensive purification of the bispecific antibodies from many hybrid-antibody side products, the presence of which may interfere with the cell cross-linking activity. The disulfide exchange method applies essentially only to F(ab′)2, and is thus limited by the susceptibility of the monoclonal antibodies to cleavage by enzyme digestion. Further, since Fab′ have little affinity for each other, very high protein concentrations are required for the formation of the inter-Fab′ disulfide bonds. The disulfide exchange method has been improved by the use of Ellman's reagent to modify one of the Fab′ prior to oxidation with the other Fab′, reducing the incidence of homodimerization. However, even with this improvement, heterodimeric F(ab′)2 can rarely be produced in better than 50% yield.

However, adverse safety issues, low response rate and limited effectiveness are general reality of the current antibody drugs. These disadvantages can be from off-target effect to normal tissues/cells because the antibody's epitope is from self antigen, inhibitory microenvironment for immune effector cells, unexpected Fc-mediated effector functions, etc. Thus, there remains a significant need for improved methods for efficiently producing bispecific antibodies at high purity.

### SUMMARY OF THE INVENTION

In one aspect, the present invention provides an engineered antibody, comprising: (i) a first polypeptide comprises a first light chain variable domain (VL1) that binds a first target and a second heavy chain variable domain (VH2) that binds a second target, wherein the VL1 is covalently linked to the VH2; and (ii) a second polypeptide comprises a second light chain variable domain (VL2) that binds the second target and a first heavy chain variable domain (VH1) that binds the first target, wherein the VL2 is covalently linked to the VH1; and wherein the VL2 and the VH2 are covalently linked, and wherein each of the VL2 and the VH2 comprises one or more substitutions that introduce charged amino acids that are electrostatically unfavorable to homodimer formation.

In some embodiments, C-terminus of the VL1 is covalently linked to N-terminus of the VH2, and C-terminus of the VL2 is covalently linked to N-terminus of the VH1. In some embodiments, N-terminus of the VL1 is covalently linked to C-terminus of the VH2, and N-terminus of the VL2 is covalently linked to C-terminus of the VH1.

In some embodiments, the VL1 is linked to the VH2 via a first peptide linker, and wherein the VL2 is linked to the VH1 via a second peptide linker. In some embodiments, the first peptide linker and the second peptide linker each independently comprises 5 to 9 amino acids.

In some embodiments, the VL2 and the VH2 are covalently linked via a disulfide bond. In some embodiments, the FR of the VL2 and FR of the VH2 are covalently linked via the disulfide bond.

In some embodiments, at least one, and preferably only one, of the residues of the FR of the VL2 is substituted with a negatively charged amino acid, and at least one, and preferably only one, of the residues of the FR of the VH2 is substituted with a positively changed amino acid. In some embodiments, at least one, and preferably only one, of the residues of the FR of the VL2 is substituted with a positively charged amino acid, and at least one, and preferably only one, of the residues of the FR of the VH2 is substituted with a negatively charged amino acid. In some embodiments, the negatively charged amino acid is aspartic acid (D) or glutamic acid (E), and the positively charged amino acid is lysine (K) or arginine (R).

In some embodiments, either of the first polypeptide and the second polypeptide is independently linked at its C terminus to a hinge region of IgG1, IgG2, IgG3, or IgG4.

In another aspect, the present invention provides an engineered antibody, comprising a dimer of the antibody provided herein, and each unit of the dimer is connected via the hinge region.

In some embodiments, either of the first polypeptide and the second polypeptide is independently linked at its C terminus to a Fc region. In some embodiments, either of the first polypeptide and the second polypeptide is independently linked at its C terminus to an albumin, or a PEG.

In a further aspect, the present invention provides an engineered antibody, comprising: (i) a first polypeptide comprises a second light chain variable domain (VL2) that binds a second target and a first heavy chain variable domain (VH1) that binds a first target, wherein the VL2 is covalently linked to the VH1; (ii) a second polypeptide comprises a first light chain variable domain (VL1) that binds the first target, a second heavy chain variable domain (VH2) that binds the second target, a hinge domain, and a CH2-CH3 domain of IgG, wherein the VL1 is covalently linked to the VH2; (iii) a third polypeptide comprises a third heavy chain variable domain (VH3) that bind a third target, a CH1domain, a cysteine-containing hinge domain, and a CH2-CH3 domain of IgG; and (iv) a fourth polypeptide comprises a fourth light chain variable domain (VL3) that binds the third target, and a cysteine-containing CL domain; wherein the VL1 and VH1 associate to form a domain capable of binding the first target; wherein the VL2 and VH2 associate to form a domain capable of binding the second target; wherein the VL3 and VH3 associate to form a domain capable of binding the third target; wherein the VL2 and the VH2 are covalently linked via a disulfide bond; wherein the VL2 and the VH2 independently comprise one or more substitutions that introduce charged amino acids that are electrostatically unfavorable to homodimer formation; wherein the CH1 and the CL are covalently linked via a disulfide bond; and wherein the second and third polypeptide chain are covalently linked via the hinge domains and the CH3 domains.

In some embodiments, the C-terminus of the VL2 is covalently linked to N-terminus of the VH1 and C-terminus of the VL1 is covalently linked to N-terminus of the VH2.

In some embodiments, the N-terminus of the VL2 is covalently linked to C-terminus of the VH1 and N-terminus of the VL1 is covalently linked to C-terminus of the VH2.

In some embodiments, the third target and the first target are the same target. In some embodiments, the third target and the second target are the same target. In some embodiments, the first target and the second target are the same target.

In some embodiments, the CH2-CH3 domain of the second polypeptide and the CH2-CH3 domain of the third polypeptide are different. In some embodiments, the second polypeptide and the third polypeptide are engineered through modification to CH3 domain interface with different mutations on each domain.

In some embodiments, one of the CH3 domains comprises a replacement of Thr366 with Trp, and the other the CH3 domain comprises a replacement of Thr366, Leu368, Tyr407 with Ser, Ala and Val respectively.

In some embodiments, one of the CH3 domains comprises a replacement of Asp399 and Glu356 with Lys, and the other the CH3 domain comprises a replacement of Lys392 and Lys409 with Asp.

In some embodiments, one of the CH3 domains comprises a replacement of Glu356, Glu357 and Asp399 with Lys, ant the other the CH3 domain comprises a replacement of Lys370, Lys409 and Lys439 with Glu, Asp and Glu respectively.

In some embodiments, one of the CH3 domains comprises a replacement of Ser364 and Phe405 with His and Ala respectively, and the other the CH3 domain comprises a replacement of Tyr349 and Thr394 with Thr and Phe respectively.

In some embodiments, one of the CH3 domains comprises a replacement of Lys370 and Lys409 with Asp, and the other the CH3 domain comprises a replacement of Glu357 and Asp399 with Lys.

In some embodiments, one of the CH3 domains comprises a replacement of Leu351 and Leu368 with Asp and Glu respectively, and the other CH3 domain comprises a replacement of Leu361 and Thr366 with Lys.

In another aspect, the present invention provides a method for treating a subject in need of treatment using an antibody provided herein.

In some embodiments, the treatment results in a sustained response in the individual after cessation of the treatment.

In some embodiments, the immunotherapeutic is administered continuously, intermittently.

In some embodiments, the individual has cancer, including colorectal cancer, melanoma, non-small cell lung cancer, ovarian cancer, breast cancer, pancreatic cancer, a hematological malignant tumor, and renal cell carcinoma, and autoimmune diseases, hematopoietic diseases, metabolic diseases, etc.

In some embodiments, wherein the antibody is administered intravenously, intramuscularly, subcutaneously, topically, orally, transdermally, intraperitoneally, intraorbitally, by implantation, by inhalation, intrathecally, intraventricularly, or intranasally.

In some embodiments, the therapeutic combination or pharmaceutical composition of the present invention further comprise an effective amount of an additional therapeutic agent, such as an anticancer agent.

In some embodiments, the anticancer agent is an antimetabolite, an inhibitor of topoisomerase I and II, an alkylating agent, a microtubule inhibitor, an antiandrogen agent, a GNRh modulator or mixtures thereof.

In some embodiments, the additional therapeutic agent is a chemotherapeutic agent selected from the group consisting of tamoxifen, raloxifene, anastrozole, exemestane, letrozole, imatanib, paclitaxel, cyclophosphamide, lovastatin, minosine, gemcitabine, cytarabine, 5-fluorouracil, methotrexate, docetaxel, goserelin, vincristine, vinblastine, nocodazole, teniposide etoposide, gemcitabine, epothilone, vinorelbine, camptothecin, daunorubicin, actinomycin D, mitoxantrone, acridine, doxorubicin, epirubicin, or idarubicin.

In another aspect, the present invention provides a method for treating a disease condition in a subject that is in need of such treatment, comprising administering to the subject the therapeutic combination or pharmaceutical composition provided herein.

In some embodiments, the diseases condition is tumor. In some embodiments, the disease condition comprises abnormal cell proliferation.

In some embodiments, the abnormal cell proliferation comprises a pre-cancerous lesion. In some embodiments, the abnormal proliferation is of cancer cells.

In some embodiments, the cancer is selected from the group consisting of: breast cancer, colorectal cancer, diffuse large B-cell lymphoma, endometrial cancer, follicular lymphoma, gastric cancer, glioblastoma, head and neck cancer, hepatocellular cancer, lung cancer, melanoma, multiple myeloma, ovarian cancer, pancreatic cancer, prostate cancer, and renal cell carcinoma.

In a further aspect, the present invention provides a kit that contains the therapeutic combination provided herein, and optionally with an instruction.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts two forms of DICAD with diagrammatical structures. Format A: the C-terminus of VL1 connects to the N-terminus of VH2 via a linker to form the first polypeptide, and the C-terminus of VL2 connects to the N-terminus of VH1 via a linker to form the second polypeptide. Format B: the C-terminus of VH2 connects to the N-terminus of VL1 via a linker to form the first polypeptide, and the C-terminus of VH1 connects to the N-terminus VL2 via a linker to form the second polypeptide.

FIG. 2 depicts structure of elements of DICAD. A: antibody variable domains used to construct DICAD. B: An example of the first and second polypeptides. C. 4 main constructions of the variable domain in DICAD regarding disulfide bond and charged amino acids introduced.

FIG. 3 depicts diagrammatically the structure of DICAD with the Fc domain (A and B) and structure of a classic antibody (C), diabody heterodimer structure (D) and scDb (single-chain diabody) (E) where C-terminus of one chain was linked to N-terminus of the other chain using a peptide of 15 amino acids.

FIG. 4A depicts diagrammatically the structure of TRIAD with Format A (Tn-specific Antibody), and the 4 polypeptides of TRIAD (Format A). FIG. 4B depicts diagrammatically the structure of TRIAD with Format B (Tri-specific Antibody), and the 4 polypeptides of TRIAD (Format B).

FIG. 4C depicts two forms of TRIAD with diagrammatical structures with polypeptides as a sequence of N-terminal to C-terminal. Format A: first polypeptide: VL2, linker, VH1; second polypeptide: VL1, linker, VH2, hinge region, CH2 and CH3; third polypeptide: VH3, CH1, hinge region, CH2 and CH3; fourth polypeptide: VL3 and CL. Format B: first polypeptide: VH2, linker, VL1; second polypeptide: VH1, linker, VL2, hinge region, CH2 and CH3; third polypeptide: VH3, CH1, hinge region, CH2 and CH3; fourth polypeptide: VL3 and CL.

FIG. 5 depicts positions of hydrogen bonds that could be changed into disulfide bonds to modify electrostatic interaction at the VH-VL interface.

FIG. 6 depicts cytotoxic effect on Raji with Jurkat depending on antibody 4, 9, 25 and 49 as measured by LDH. FIG. 6A: hollow square: antibody 25; solid circle: antibody 4; solid square: antibody 9. FIG. 6B: solid circle: antibody 25; solid square: antibody 49.

FIG. 7 depicts cytotoxic effect of Jurkat cells on Raji cells mediated by TRIAD antibodies 50 and 54 as assayed by LDH release. solid circle: antibody #54; solid triangle: antibody #50.

FIG. 8 depicts gating strategies adopted in the Raji killing assays for calculation of the absolute number of remaining CFSE-stained Raji cells.

FIG. 9 depicts antibody-mediated cytotoxic effect of PBMC, CD4+ and CD8+ on Raji cells depicted in percentage of Raji cells undergoing apoptosis induced by antibody #25 at 1, 100 pM concentration after 4 h(A), 20 h(B) and 40 h(C) co-incubation.

FIG. 10 depicts antibody-mediated cytotoxic effect of PBMC, CD4+ and CD8+ on Raji cells depicted in fold of killing effect induced by antibody #25 at 1, 100 pM concentration after 4 h(A), 20 h(B) and 40 h(C) co-incubation; for each cell group (PBMC etc.), samples without antibody (concentration=0) were used as control thus fold=1.

FIG. 11 depicts antibody-mediated cytotoxic effect of PBMC, CD4+ and CD8+ on Raji cells depicted in absolute count of remaining live Raji cells after killing induced by antibody #25 at 1, 100 pM concentration after 4 h, 20 h and 40 h co-incubation.

FIG. 12 depicts antibody-mediated cytotoxic effect of PBMC, CD4+ and CD8+ on Raji cells depicted in LDH secretion induced by antibody #25 at 1, 100 pM concentration after 4 h, 20 h and 40 h co-incubation.

FIG. 13 depicts tumor inhibition effect of antibodies on Jeko-1 xenograft model in Nod-SCID mice as measured by the volume of tumors. Hollow circle: vehicle, intravenous injection, twice weekly for 3 weeks; hollow triangle(up): antibody 49, 0.5 mg/kg intravenous injection, once per day for 10 days; hollow triangle(down): antibody 1, 0.5 mg/kg intravenous injection, twice weekly for 3 weeks; hollow diamond: antibody 25, 0.5 mg/kg intravenous injection, twice weekly for 3 weeks; solid diamond: antibody 50, 0.5 mg/kg intravenous injection, twice weekly for 3 weeks; solid square: antibody 54, 0.5 mg/kg intravenous injection, twice weekly for 3 weeks.

FIG. 14 depicts diagrammatically the structure of DICAD constructed according to another example as described herein.

FIG. 15 depicts the killing effect of sample antibody #63—CD3×CD19 bispecific, Blinatumomab, MGD011 and RG6026 on target cells.

FIG. 16 depicts tumor inhibition effect of sample antibody #63—CD3×CD19 bispecific, Blinatumomab, MGD011 and RG6026.

FIG. 17 depicts effect of sample antibody #63—CD3×CD19 bispecific, Blinatumomab, MGD011 and RG6026 on mice body weight.

FIG. 18 depicts the killing effect of sample antibody #63—CD3×CD19 and sample antibody #55—CD3×CD19×CD8 on Raji cells.

FIG. 19 depicts effect of sample antibody #63—CD3×CD19 and sample antibody #55-CD3×CD19×CD8 at various concentrations on mice body weight.

FIG. 20 depicts tumor inhibition effect of sample antibody #63—CD3×CD19 and sample antibody #55—CD3×CD19×CD8 at various concentrations.

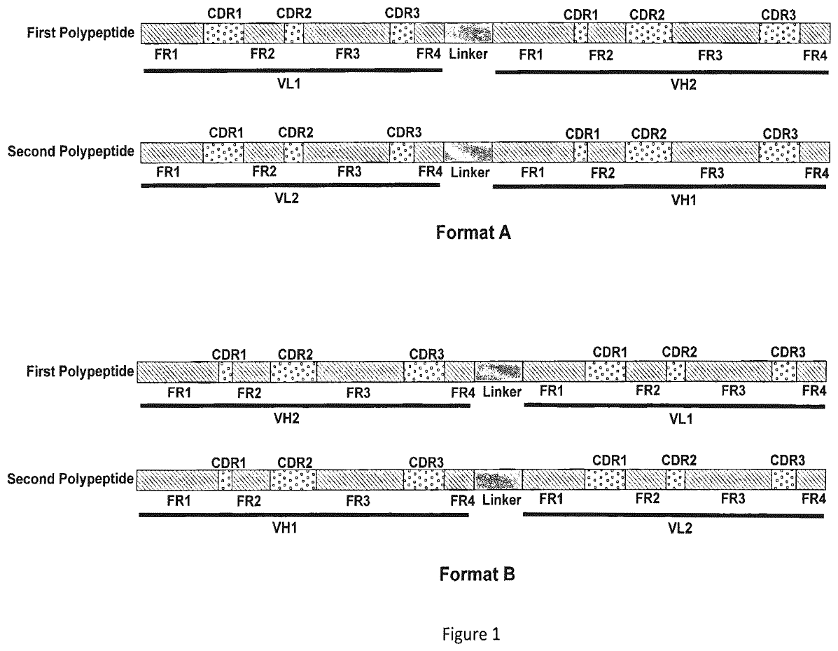

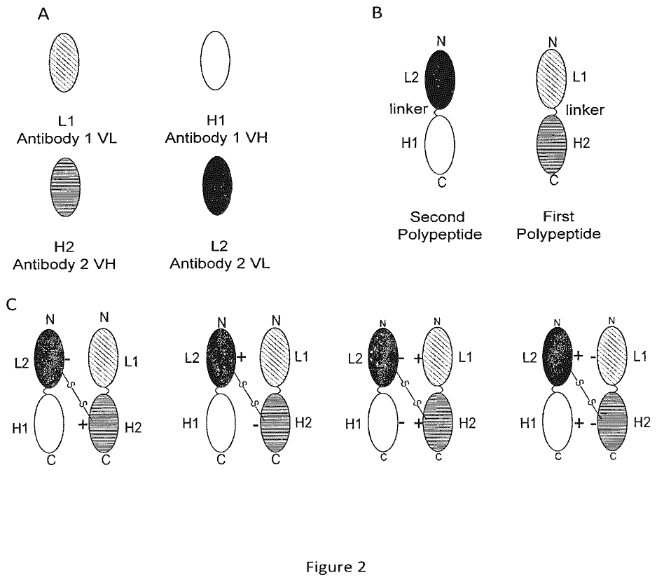

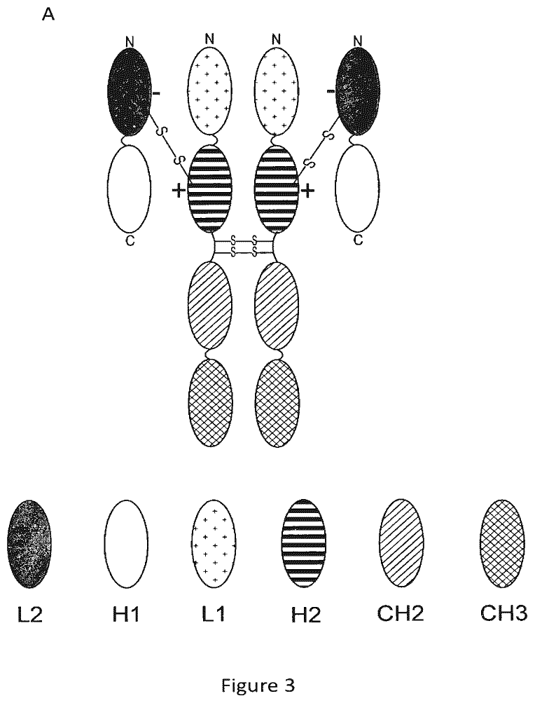

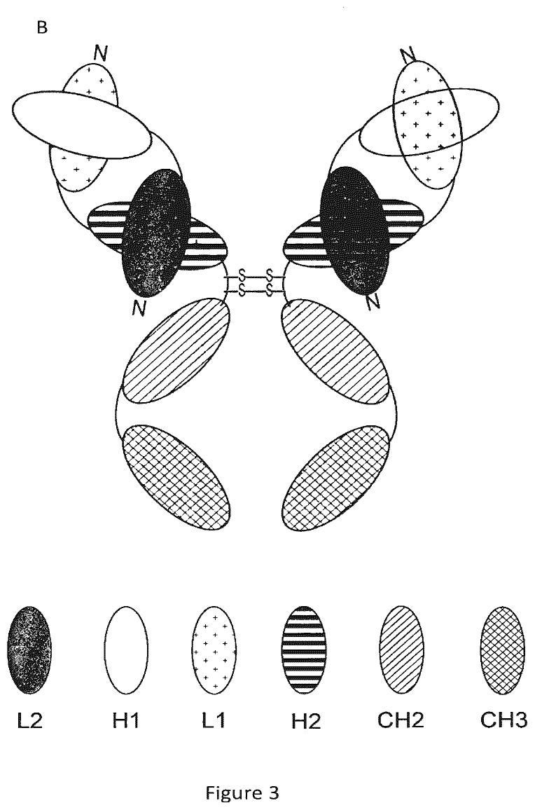

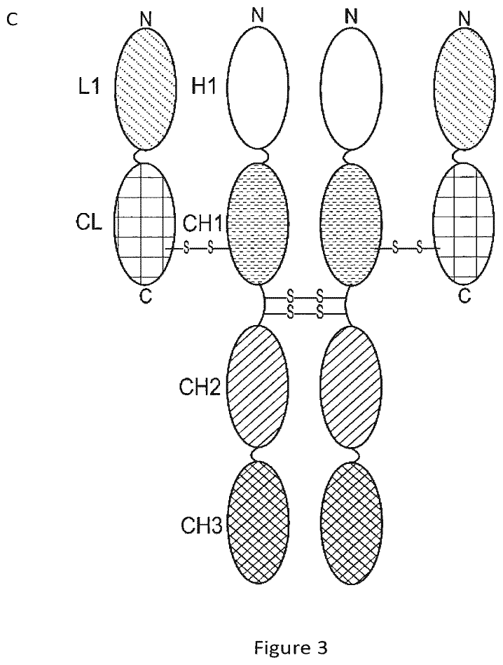

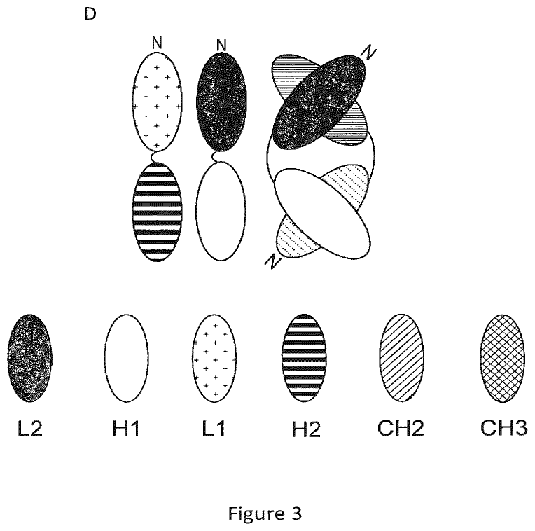

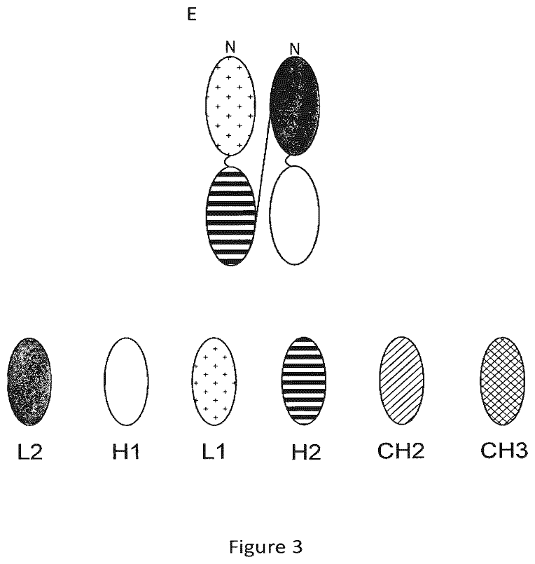

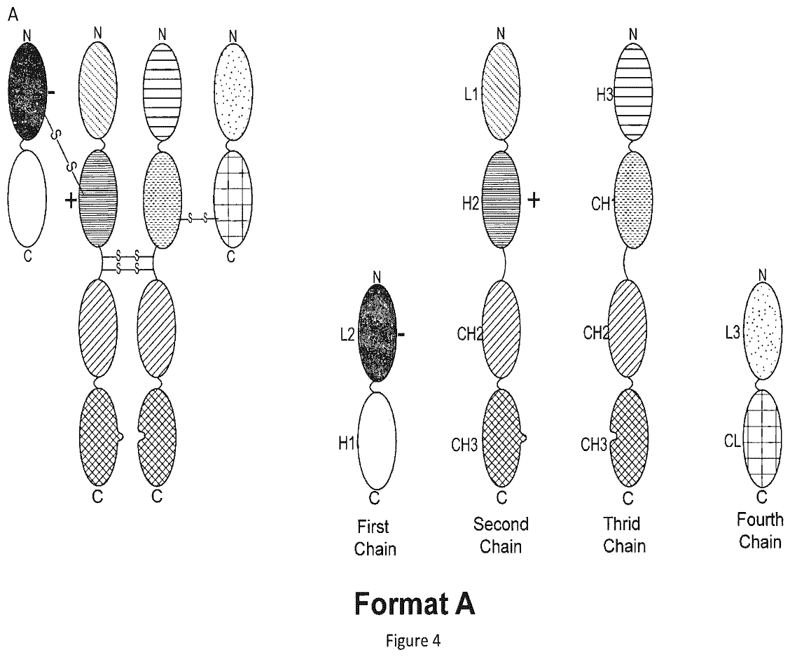

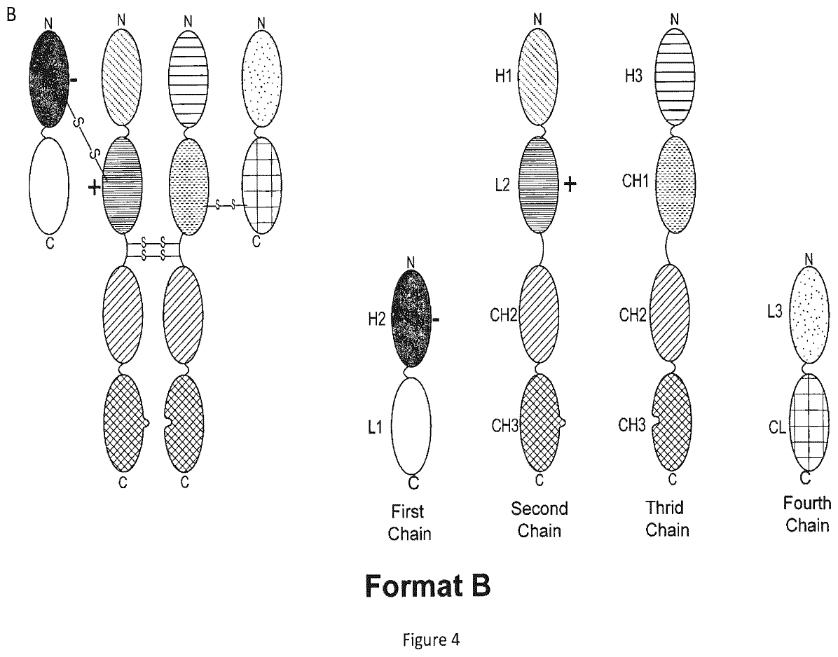

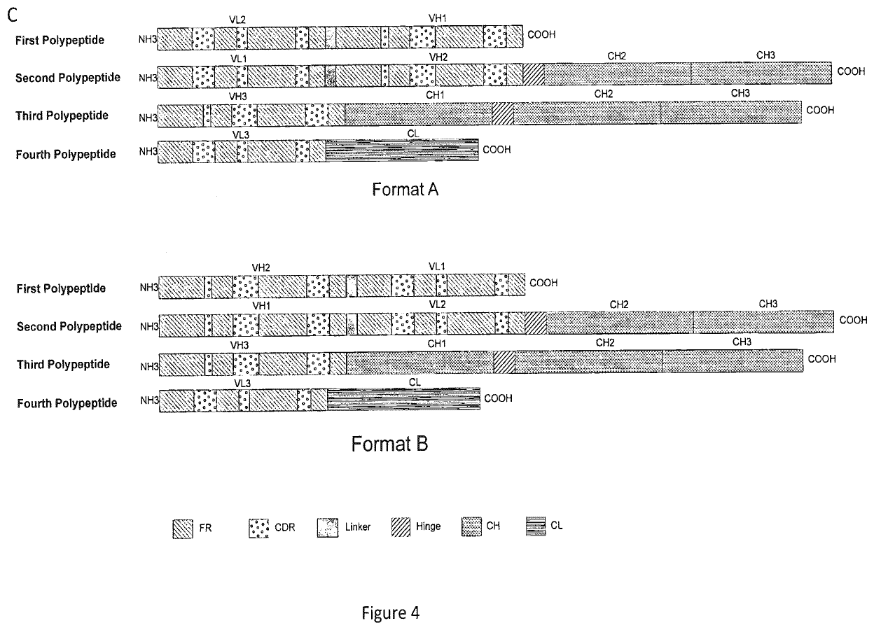

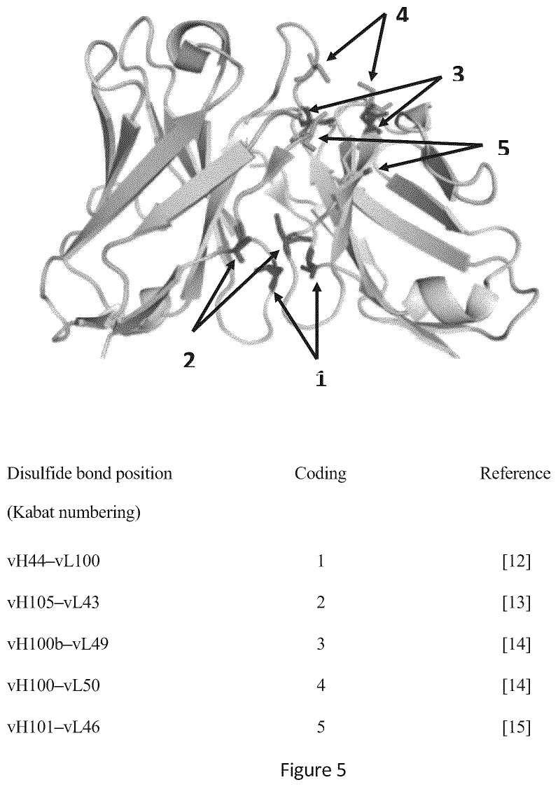

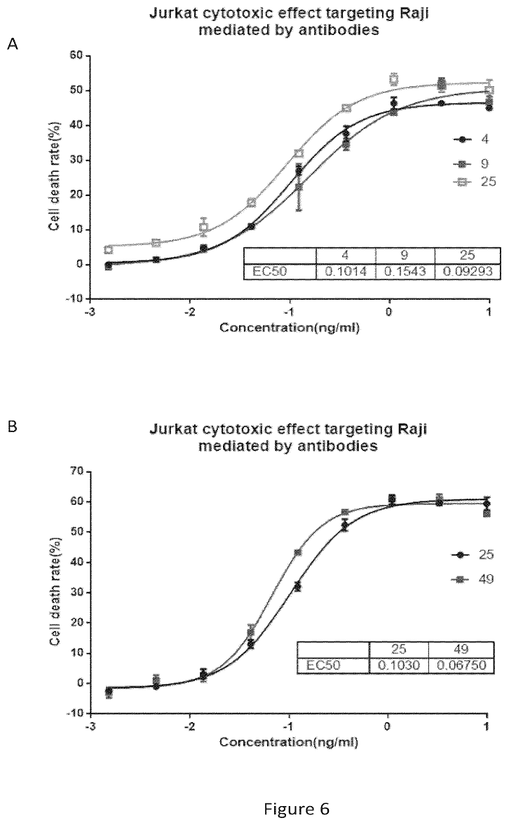

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events.

Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

### I. DEFINITIONS AND ABBREVIATIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well-known and commonly employed in the art. Standard techniques are used for nucleic acid and peptide synthesis. The techniques and procedures are generally performed according to conventional methods in the art and various general references, which are provided throughout this document. The nomenclature used herein and the laboratory procedures in analytical chemistry, and organic synthetic described below are those well-known and commonly employed in the art. Standard techniques, or modifications thereof, are used for chemical syntheses and chemical analyses.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the disclosure.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The terms “polypeptide”, “peptide”, and “protein” are used interchangeably herein to designate a linear series of amino acid residues connected one to the other by peptide bonds, which includes proteins, polypeptides, oligopeptides, peptides, and fragments thereof. The protein may be made up of naturally occurring amino acids and/or synthetic (e.g. modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Furthermore, it should be noted that a dash at the beginning or end of an amino acid residue sequence indicates either a peptide bond to a further sequence of one or more amino acid residues or a covalent bond to a carboxyl or hydroxyl end group. However, the absence of a dash should not be taken to mean that such peptide bonds or covalent bond to a carboxyl or hydroxyl end group is not present, as it is conventional in representation of amino acid sequences to omit such.

By “nucleic acid” herein is meant either DNA or RNA, or molecules which contain deoxy- and/or ribonucleotides. Nucleic acid may be naturally occurring or synthetically made, and as such, includes analogs of naturally occurring polynucleotides in which one or more nucleotides are modified over naturally occurring nucleotides.

The terms “conjugated” and “joining” generally refer to a chemical linkage, either covalent or non-covalent that proximally associates one molecule with second molecule.

The term “isolated” is intended to mean that a compound is separated from all or some of the components that accompany it in nature. “Isolated” also refers to the state of a compound separated from all or some of the components that accompany it during manufacture (e.g., chemical synthesis, recombinant expression, culture medium, and the like).

The term “purified” is intended to mean a compound of interest has been separated from components that accompany it in nature or during manufacture and provided in an enriched form.

The “potent” or “potency” used in the context of a compound herein refers to ability or capacity of the compound to exhibit a desired activity.

The term “concentration” used in the context of a molecule such as peptide fragment refers to an amount of molecule present in a given volume. In some embodiments, a concentration of a molecule is given in a molar concentration where the number of moles of the molecules present in a given volume of solution is indicated.

The terms “antigen” and “epitope” interchangeably refer to the portion of a molecule (e.g., a polypeptide) which is specifically recognized by a component of the immune system, e.g., an antibody. As used herein, the term “antigen” encompasses antigenic epitopes, e.g., fragments of an antigen which are antigenic epitopes.

The term “antibody” encompasses polyclonal and monoclonal antibody where the antibody may be of any class of interest (e.g., IgG, IgM, and subclasses thereof), as well as hybrid antibodies, altered antibodies, F(ab′)2 fragments, F(ab) molecules, Fv fragments, single chain fragment variable displayed on phage (scFv), single chain antibodies, single domain antibodies, diabodies, chimeric antibodies, humanized antibodies, and a fragment thereof. In some embodiments, the fragments of an antibody may be functional fragments which exhibit immunological binding properties of the parent antibody molecule. The antibodies described herein can be detectably labeled, e.g., with a radioisotope, an enzyme which generates a detectable product, a fluorescent protein, and the like. Detectable labels that find use in in vivo imaging are of interest. The antibodies may be further conjugated to other moieties, such as a cytotoxic molecule or other molecule, members of specific binding pairs, and the like.

A typical antibody structural unit, especially when it is in full length, is known to include a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively.

An “antigen-binding site” or “binding portion” refers to the part of an antibody molecule or fragment thereof that participates in antigen binding. The antigen binding site is formed by amino acid residues of the N-terminal variable heavy chain (VH) and variable light chain (VL). Three highly divergent stretches within the variable regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three-dimensional space to form an antigen binding “surface”. This surface mediates recognition and binding of the target antigen. The three hypervariable regions of each of the heavy and light chains are referred to as “complementarity determining regions” or “CDRs”. The CDRs are primarily responsible for binding to an epitope of an antigen.

Antibody and fragments thereof according to the present disclosure encompass bispecific antibodies and fragments thereof. Bispecific antibodies may resemble single antibodies (or antibody fragments) but have two different antigen binding sites. Bispecific antibodies may have binding specificities for at least two different epitopes. Bispecific antibodies and fragments can also be in form of heteroantibodies. Heteroantibodies are two or more antibodies, or antibody binding fragments (e.g., Fab) linked together, each antibody or fragment having a different specificity.

Antibody conjugates are also provided. The conjugates include any antibody of the present disclosure and an agent. The agent may be selected from a therapeutic agent, an imaging agent, a labeling agent, or an agent useful for therapeutic and/or labeling purposes.

The strength or affinity of immunological binding interactions between an antibody (or fragment thereof) and the specific antigen (or epitope) can be expressed in terms of the dissociation constant (Kn) of the interaction, wherein a smaller Kn represents a greater affinity. Immunological binding properties of selected polypeptides can be quantified using methods well known in the art. One such method entails measuring the rates of antigen binding site/antigen complex formation and dissociation, wherein those rates depend on the concentrations of the complex partners, the affinity of the interaction, and on geometric parameters that equally influence the rate in both directions. Thus, both the “on rate constant” (kₒₙ) and the “off rate constant” (k_{off}) can be determined by calculation of the concentrations and the actual rates of association and dissociation. The ratio of kofflkon enables cancellation of all parameters not related to affinity and is thus equal to the equilibrium dissociation constant K_{D} (see, generally, Davies et al. Ann. Rev. Biochem. 1990, 59: 439-15 473).

The term “specific binding of an antibody” or “antigen-specific antibody” in the context of a characteristic of an antibody refers to the ability of an antibody to preferentially bind to a particular antigen that is present in a mixture of different antigens. In certain embodiments, a specific binding interaction will discriminate between desirable and undesirable antigens (or “target” and “non-target” antigens) in a sample, in some embodiments more than about 10 to 100-fold or more (e.g., more than about 1000- or 10,000-fold). In certain embodiments, the affinity between an antibody and antigen when they are specifically bound in an antibody antigen complex is characterized by a K_{D} (dissociation constant) of less than 10⁻⁶M, less than 10⁻⁷ M, less than 10⁻⁸ M, less than 10⁻⁹ M, less than 10⁻⁹ M, less than 10⁻¹¹ M, or less than about 10⁻¹² M or less.

The term “monoclonal antibody” refers to an antibody composition having a homogeneous antibody population. The term encompasses whole antibody molecules, as well as Fab molecules, F(ab′)2 fragments, Fv fragments, single chain fragment variable displayed on phage (scFv), fusion proteins comprising an antigen-binding portion of an antibody and a non-antibody protein, and other molecules that exhibit immunological binding properties of the parent monoclonal antibody molecule. Methods of making and screening polyclonal and monoclonal antibodies are known in the art.

The terms “derivative” and “variant” refer to without limitation any compound or antibody which has a structure or sequence derived from the compounds and antibodies of the present disclosure and whose structure/sequence is sufficiently similar to those disclosed herein and based upon that similarity, would be expected, by one skilled in the art, to exhibit the same or similar activities and utilities as the claimed and/or referenced compounds or antibody, thereby also interchangeably referred to “functional equivalent”. Modifications to obtain “derivative” or “variant” includes, for example, by addition, deletion and/or substitution of one or more of the amino acid residues. The functional equivalent or fragment of the functional equivalent may have one or more conservative amino acid substitutions. The term “conservative amino acid substitution” refers to substitution of an amino acid to another amino acid that has similar properties to the original amino acid. The groups of conservative amino acids are known in the art.

Conservative substitutions may be introduced in any position of a preferred predetermined peptide or fragment thereof. It may however also be desirable to introduce nonconservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

“Percentage of sequence identity” is determined by comparing two optimally aligned sequences over a comparison window, wherein the portion of the polynucleotide or polypeptide sequence in the comparison window may have additions or deletions (i.e., gaps) as compared to the reference sequence (which does not have additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences or individual domains of the polypeptides), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 5 to 50 nucleotides or polypeptide sequences in length, or more preferably over a region that is 100 to 500 or 1000 or more nucleotides or polypeptide sequences in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full-length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482, by the homology alignment algorithm of Needleman and Wunsch (1970) J Mol. Biol. 48:443, by the search for similarity method of Pearson and Lipman (1988) Proc. Natl. Acad Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information (http://www.ncbi.nlm.nih.gov/).

“Cell(s) of interest” or “target cell(s)” used herein interchangeably refers to a cell or cells where one or more signaling pathways are intended to modulated. In some embodiments, the target cell(s) includes, but not limited to, a cancer cell(s). In some other embodiments, the target cell(s) includes immune effector cells such as natural killer cell(s), T cell(s), dendritic cell(s) and macrophage(s).

A “cancer cell” as used herein refers to a cell exhibiting a neoplastic cellular phenotype, which may be characterized by one or more of, for example, abnormal cell growth, abnormal cellular proliferation, loss of density dependent growth inhibition, anchorage independent growth potential, ability to promote tumor growth and/or development in an immunocompromised non-human animal model, and/or any appropriate indicator of cellular transformation. “Cancer cell” may be used interchangeably herein with “tumor cell” or “cancerous cell”, and encompasses cancer cells of a solid tumor, a semi-solid tumor, a primary tumor, a metastatic tumor, and the like.

By “treatment” in the context of disease or condition is meant that at least an amelioration of the symptoms associated with the condition afflicting an individual is achieved, where amelioration is used in a broad sense to refer to at least a reduction in the magnitude of a parameter, e.g. symptom, associated with the condition (e.g., cancer) being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease, e.g., so as to decrease tumor load, which decrease can include elimination of detectable cancerous cells, or so as to protect against disease caused by bacterial infection, which protection can include elimination of detectable bacterial cells; and/or (iii) relief, that is, causing the regression of clinical symptoms.

The term “effective amount” of a composition as provided herein is intended to mean a non-lethal but sufficient amount of the composition to provide the desired utility. For instance, for eliciting a favorable response in a cell(s) of interest (“target cell(s)”) such as modulating a signaling pathway, the effective amount of an (active, effective, potent or functional) antibody is the amount which results in notable and substantial change in the level of the activity of the signaling pathway, including downregulation and upregulation of the signaling pathway, when compared to use of no antibody or a control (inactive, ineffective, or non-functional) antibody. The measurement of changes in the level of the activity of the signaling pathway can be done by a variety of methods known in the art. In another example, for eliciting a favorable response in a subject to treat a disease (e.g., cancer), the effective amount is the amount which reduces, eliminates or diminishes the symptoms associated with the disorder, e.g., so as to provide for control of cancer metastasis, to eliminate cancer cells, and/or the like. As well be understood by a person having ordinary skill in the art, the exact amount required will vary from subject to subject, depending on the species, age, and general condition of the subject, the severity of the condition or disease that is being treated, the particular composition used, its mode of administration, and the like. Thus, it is not possible to specify an exact “effective amount.” However, an appropriate effective amount may be determined by one of ordinary skill in the art using only routine experimentation.

The term “pharmaceutically acceptable excipient” as used herein refers to any suitable substance which provides a pharmaceutically acceptable compound for administration of a compound(s) of interest to a subject. “Pharmaceutically acceptable excipient” can encompass substances referred to as pharmaceutically acceptable diluents, pharmaceutically acceptable additives and pharmaceutically acceptable carriers.

The terms “individual” or “subject” are intended to cover humans, mammals and other animals. The terms “individual” or “subject” are used interchangeably herein to refer to any mammalian subject to whom antibodies or fragments thereof in the present disclosure is subjected.

Certain embodiments feature a bispecific antibody, antigen binding fragment, or recombinant protein thereof, which is capable of modulating of the activity of one or more signaling pathway in a cell or cells of interest. The modulation of the one or more signaling pathway may lead to certain changes in target cell(s)'s behavior, such as stimulating or reducing cell proliferation, cell growth, cell differentiation, cell survival, cell secretion, modulation of adhesion and/or motility of cells.

As used herein, the term “pharmaceutically acceptable salts” refers to salts that retain the biological effectiveness and properties of the compounds of this invention and, which are not biologically or otherwise undesirable. In many cases, the compounds of the present invention are capable of forming acid and/or base salts by virtue of the presence of amino and/or carboxyl groups or groups similar thereto (e.g., phenol or hydroxyamic acid). Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985), which is herein incorporated by reference.

As used herein, the term “pharmaceutically acceptable carrier/excipient” includes any and all solvents, dispersion media, coatings, surfactants, antioxidants, preservatives (e.g., antibacterial agents, antifungal agents), isotonic agents, absorption delaying agents, salts, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except in so far as any conventional carrier is incompatible with the active ingredient, its use in the therapeutic or pharmaceutical compositions is contemplated.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “therapeutic combination” or “combination” refers to a combination of one or more active drug substances, i.e., compounds having a therapeutic utility. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen.

### II. COMPOSITIONS

In general, the present invention provides a DICAD (Disulfide and Charge Adjusted Diabody) platform that is designed on basis of the Diabody technology. By introducing covalent bonds and adjusting amino acids to modify electrostatic charges at the VH-VL interface, this new platform is able to produce multivalent antibodies with two or more specificities. With stability and pharmacokinetic properties analogue to classic antibodies, DICADs can be efficiently expressed and purified using traditional procedures. The antibodies proved to have very high potency both in vitro and in vivo, and have a long half-life compared to most other bi-specific antibodies.

In general, the antibodies provided herein have a structure that was designed based on Diabody. They have a disulfide bonding between VH and VL, as well as mutation on selected amino acid based on their electrostatic properties. For example, some diabodies have modifications at VL2-VH2, which can also be VL1-VH1, or both. Both to improve association and desired pairing of VH and VL.

In some embodiments, there are both VL1-VH1 and VL2-VH2, purity of product will increase but yield will decrease, thus may not be preferred.

In some embodiments, an Fc fragment with knob-into-hole domain is included.

In general, the antibodies provided herein (DICADs) resemble classic antibody. They are more stable, have a longer half-life, and are easy for downstream purification.

In general, the antibodies provided herein have the following advantages: (1) retain the properties of bivalent bi-specific antibodies: avidity, affinity, potency etc.; (a) have high stability and less aggregation; (3) are easy for expression and purification, in comparison to other bi-specific antibodies; and (4) have a structure that similar to native IgG, and thus have decreased immunogenicity.

### A. Disulfide and Charge Adjusted Diabody

In one aspect, the present invention provides antibodies, such as disulfide and charge adjusted diabodies (DICADs).

The present invention provides an engineered antibody, comprising: (i) a first polypeptide comprises a first light chain variable domain (VL1) that binds a first target and a second heavy chain variable domain (VH2) that binds a second target, wherein said VL1 is covalently linked to said VH2; and (ii) a second polypeptide comprises a second light chain variable domain (VL2) that binds said second target and a first heavy chain variable domain (VH1) that binds said first target, wherein said VL2 is covalently linked to said VH1; and wherein said VL2 and said VH2 are covalently linked, and wherein each of said VL2 and said VH2 comprises one or more substitutions that introduce charged amino acids that are electrostatically unfavorable to homodimer formation.

### Diabody

The antibodies provided herein have various superior properties in comparison to other common form of diabodies.

Diabody is an scFv (single chain Fv) heterodimer structure, which was reported by Holliger et al as early as in 1993 (1). Either chain was constructed with one VL and one VH originated from Fvs of different antibodies respectively, linked via a peptide of 5-10 amino acids. Short length of the linker peptides brings the fragments to close proximity, together with affinity between fragments, drive scFvs to dimerize into molecules of 55-60 kDa that are capable of recognizing two antigens simultaneously. Later the system had been further modified and optimized; its core fundamental structure and methods for construction were thus established (2, 3). Diabodies have been shown to have very high affinity for their targets. However, the structure lacks inter-fragment covalent binding or the Fc regions, resulting in poor stability and short half-life of the molecules, which make diabodies far from a mature and qualified industry product.

To improve druggability of diabodies, Zhu and colleagues added an Fc region to C terminus of each chain of the diabody through a hinge, and the modified structure then dimerized and resulted in a Di-diabody (54, 5). Comparing to Diabodies, Di-diabodies have significantly longer half-life in vivo and much more simplified purification procedures, owing to their Fc regions. The new structure also retained bivalent binding to individual antigens and was able to maintain most of avidity and selectivity of the original (mono-specific) antibodies. All made Di-diabodies better resemble traditional antibodies regarding their pharmacokinetic properties.

But stability remained a problem for Di-diabody, as it was for Diabody. Once in vivo, di-diabodies rapidly lost their activities due to dissociation of the dimers. Also, concentration and proportion of undesirable side production in serum were very difficult to determine by regular experimental methods such as ELISA, casting a shadow on the druggability study.

To improve homogeneity of Diabody products, Kontermann et al developed the scDB (single-chain diabody) platform (6, 7, 8, 9). C-terminus of one chain was linked to N-terminus of the other chain using a peptide of 15 amino acids. This change enhanced the stability of the original diabodies and improved homogeneity of the product. Kumagai et al at Tohoku University further modified the scDb system by linking the N-terminus of an Fc region to the C-terminus of a Diabody peptide via a hinge structure to improve the expression/purification process and increase product in vivo half-life. The modified system largely maintained affinity and selectivity of the original antibodies, though there was inevitable loss of affinity in the distal end of the HC arm. However, under this type of construction the only connection between two pairs of VL-VH peptides depended on the linker peptide that was flexible, which allowed aggregation of the molecules and resulted in poor stability. Moreover, these aggregates usually beared higher immunogenicity and tended to induce ADAs (anti-drug antibodies) in vivo, posing big challenges for formulation.

### Various Format of Disulfide and Charge Adjusted Diabody (DICAD)

The antibodies provided herein can have various formats or structures.

In some embodiments, C-terminus of the VL1 is covalently linked to N-terminus of the VH2, and C-terminus of the VL2 is covalently linked to N-terminus of the VH1.

In some embodiments, the VL1 is linked to the VH2 via a first peptide linker, and the VL2 is linked to the VH1 via a second peptide linker.

In some embodiments, the first polypeptide has the following structure from N terminus to C terminus:
VL1-First Peptide Linker-VH2.

In some embodiments, the second polypeptide has the following structure from N terminus to C terminus:
VL2-Second Peptide Linker-VH1.

In some embodiments, N-terminus of said VL1 is covalently linked to C-terminus of the VH2, and N-terminus of the VL2 is covalently linked to C-terminus of the VH1.

In some embodiments, the the first polypeptide has the following structure from N terminus to C terminus:
VH2-First Peptide Linker-VL1.

In some embodiments, the second polypeptide has the following structure from N terminus to C terminus:
VH1-Second Peptide Linker-VL2.

In some embodiments, the first peptide linker and the second peptide linker each independently comprises 1 to 15, 20, or 30, and preferably 5 to 9 amino acids.

In some embodiments, the linker has a sequence of: RTVAA (SEQ ID NO.:1), GGGGS (SEQ ID NO.:2), GGSGGS (SEQ ID NO.:3), GGSGGSGGS (SEQ ID NO.:4), or GGGGSGGGGS (SEQ ID NO.:5).

### Disulfide Bond

The antibodies provided herein generally comprises a covalent link to link the polypeptides.

In some embodiments, the VL2 and the VH2 are covalently linked via a disulfide bond.

In some embodiments, FR of the VL2 and FR of the VH2 are covalently linked via said disulfide bond.

Analysis on crystal structure of antibodies revealed that cysteine mutations could be introduced in some of the relatively conserved sequences at the VL-VH interface to form disulfide bonds between VL and VH, so they are covalently connected. Covalent bonds between VLs and VHs significantly improved stability of the antibodies, as for both scDb and Diabody; level of aggregation was also decreased in the latter.

The initial dsFv (disulfide Fv) was constructed by introducing disulfide bonds to VH-VL interface via covalent interaction between cysteine residues in CDRs of each fragment respectively (20). Though activity of antibodies was not affected using this method, detailed structure information on the CDRs of the original antibodies was required for “customized” design to avoid interference with antigen-recognizing/binding capability of the CDRs, which made it difficult for the method to become a universal solution for construction of various antibodies. To ensure broad application of the method, it is crucial that only amino acids at selected sites in conserved FR are engaged in the construction of dsFv.

TABLE 1
Summary of disulfide bond position

| Disulfide bond position (Kabat numbering) | Reference |
|---|---|
| vH44-vL100 | [12] |
| vH105-vL43 | [13] |
| vH100b-vL49 | [14] |
| vH100-vL50 | [14] |
| vH101-vL46 | [15] |

Since 1993, several paired sites for VH-VL covalent bond formation have been discovered, including VH44-VL100, VH105-VL43, VH100b-VL49 and VH100-VL150 etc. (ref 12,13,14,15). Among them VH44-VL100 and VH105-VL43 are, to different extent, superior to the others in many aspects such as protein expression level, mono rate, Tm and affinity etc., and are thus subjected to broader application. In the process of construction of DICAD, we have also come to the realization that VH44-VL100 has clear advantages over its peer.

In some embodiments, the VL1 and the VH1 are covalently linked via a disulfide bond, wherein the disulfide bond links FR4 of the VL1 to FR2 of the VH1.

In some embodiments, position 100 of the VL1 (Kabat) and position 44 of the VH1 (Kabat) are substituted with cystine.

In some embodiments, the VL1 and said VH1 are covalently linked via a disulfide bond, wherein the disulfide bond links FR2 of the VL1 to FR4 of the VH1.

In some embodiments, position 43 of said VL1 (Kabat) and position 105 of the VH1 (Kabat) are substituted with cystine.

The substituted cystines form disulfide bonds that linked the heavy chains and the lights chains of the antibodies provided herein.

Substitution with Charged Amino Acids

In another aspect, the antibodies provided here comprise one or more amino acids substitutions with different charge properties that results in superior properties.

In some embodiments, FR of the VL2 is substituted with a negatively charged amino acid, and FR of the VH2 is substituted with a positively changed amino acid.

In some embodiments, FR of the VL2 is substituted with a positively charged amino acid, and FR of the VH2 is substituted with a negatively charged amino acid.

Formation of covalent interaction in between the two chains of Diabody by introducing disulfide bonds could significantly improve homogeneity and stability of the product. Our earlier research did show that introduction of disulfide bonds substantially increased the mono rate. However, there was always a portion of heavy and light chains paired up in a mistaken, non-covalent manner. Covalent interaction alone did not guarantee purity of the production. Based on the finding, we revised the system and took into consideration the influence of regional electrostatic forces, to improve purity of the product.

In folded water-soluble polypeptides, side chains of hydrophobic amino acids cluster together within the structure, forming “hydrophobic cores” that are buries from water. Meanwhile, side chains of hydrophilic amino acids are situated on the solvent-exposed surface where they interact with surrounding water molecules. Hydrophobic cores, together with hydrophilic surfaces, drive the folding of water-soluble polypeptides. Exposure of hydrophobic amino acids on surface of the polypeptide increase entropy and free energy thus destabilized the structure and vice versa. Similar hydrophobic interactions exist in between VH and VL of an antibody, and residues involved are relatively conserved: H37, H45, H47, H89, H91, H104 from FR of the heavy chain and L36, L44, L46, L85, L87, L98 from FR of the light chain. Side chains from these amino acids cluster and form a hydrophobic core, in addition to a hydrogen bond formed in between two glutamines at H39 and L38, respectively. Both H39 and L38 are fairly conserved residues: in human H39 is a Q 95% of the time, and the frequency is 94% for KVL and 95% for λVL. By replacing glutamine residues at H39 and L38 with selected hydrophobic/hydrophilic residues, we were able to promote desired VH-VL pairing while at the same time inhibit undesired association.

Tan et al (16) managed to influence stability of the scFv (single chain F variant) by adjusting amino acids at VH-VL interface based on their electrostatic properties. Later, Igawa (21,22) et al adapted the method to modify scDb. Two pairs of Q39-Q38 in 4 V fragments respectively were replaced with amino acids with proper electrostatic charge to either promote or inhibit certain isoforms, in order to improve homogeneity of the product. Similar method was applied to Fc arms of the antibodies: modified electrostatic properties at the CH3 interface promoted interaction between homogeneous CH3 domains (21, 22). Gunasekaran et al at Amgen did further research on the method and engaged it in modification of the Fab arms of antibodies. Adjusting electrostatic steering at the CH1-CL interface, together with modifications at 38-39 of VH-VL, facilitated specific interaction between CH1-VH and CL-VL (23, 24). Each HC was thus able to interact with two LCs and resulted in antibodies that could bind to two antigens at the same time.

The DICAD provided herein modified electrostatic steering of selected regions in addition to insertion of disulfide bonds, and by doing so managed to minimize unwanted non-specific interactions. This platform improved pharmacokinetic properties of the molecules, helped to remove obstacles in downstream development process, and increased probability of success in development of bi-specific antibodies.

W103 of VH and P44 of VL are both at the side chain of the hydrophobic core and positioned in close proximity. Electrostatic interaction between W103-P44 was also examined during development of DICAD and found to be superior.

In some embodiments, the FR2 of the VL1 is substituted with a negatively charged amino acid, and FR2 or FR4 of the VH1 is substituted with a positively changed amino acid.

In some embodiments, the FR2 of the VL1 is substituted with a positively charged amino acid, and FR2 or FR4 of the VH1 is substituted with a negatively changed amino acid.

In some embodiments, the negatively charged amino acid is aspartic acid (D) or glutamic acid (E), and the positively charged amino acid is lysine (K) or arginine (R).

In some embodiments, the FR2 of the VL1 is substituted at P44, and the FR4 of the VH1 is substituted at W103.

In some embodiments, the FR2 of said VL1 is substituted at Q38, and said FR2 of said VH1 is substituted at Q39.

Introduction of positively or negatively charged amino acid into an antibody are known in the art.

### Additional Features

In another aspect, the present invention provides an engineered antibody, comprising a dimer of the antibody provided herein and each unit of said dimer is connected via a hinge region.

In some embodiments, either of the first polypeptide and the second polypeptide is independently linked at its C terminus to a hinge region of IgG1, IgG2, IgG3, or IgG4.

In some embodiments, either of the first polypeptide and the second polypeptide is independently linked at its C terminus to a hinge region and CH2-CH3 of IgG1, IgG2, IgG3, IgG4, or IgA, to form a classic antibody like homodimer.

The hinge region that links the Fc and DICAD portions of the antibody molecule is in reality a flexible tether, allowing independent movement of the two DICAD arms.

In some embodiments, either of the first polypeptide and the second polypeptide is independently linked at its C terminus to a Fc region.

In some embodiments, either of the first polypeptide and the second polypeptide is independently linked at its C terminus to an albumin, or a PEG.

In some embodiments, the the molecular weight of PEG is about 1 to 40 KDa.

### B. Trivalent Antibodies

In another aspect, the present invention provides TRIAD (Tri-specific Adjusted Diabody), a platform for construction of antibodies with molecular weight of about 153 kDa and that were able to simultaneously recognized 3 antigens. It was developed on the basis of DICAD, through a series of modifications adopting the “knob-into-hole” technology.

Briefly, the CH3 domain in the polypeptide containing the Fc segment was modified into a “knob” structure, the CH3 domain of the y chain of a traditional antibody was modified into a “hole” structure, then co-expressed with a light chain (LC) from a traditional antibody: all together to realize the construction of TRIAD antibodies. Point mutations at multiple sites were engaged and screened for further optimization, and thus determined the structure and construction method of TRIADs. Through addition of a third antigen-recognizing functional domain, we were able to realize either AAB (2:1) or ABC (1:1:1) manner (A, B, and C each represent a target of selection) in target binding, which resulted in MOA and pharmacokinetic properties different from that of DICADs.

The AAB (2:1) type of construction consists of two pairs of VH1-VL1 that target antigen A, and one pair of VH2-VL2 that targets antigen B. Bi-covalent interaction between CD3 antibodies and T cells induces T cell apoptosis and greatly increases clinical CRS risk due to large release of cytokines. Thus, to lower CRS risk, single valent interaction for CD3 antibody is always adopted when constructing T cell-engaged antibodies. The bi-valent interaction for the other antigen increased avidity of the antibody for the antigen and resulted in two advantages: (1) the antibody is able to recognize antigens with low abundance when single chain antibody has high affinity for the antigen; and (2) the antibody is highly selective when interacting with antigens, i.e. it only binds to antigens with high abundance but not to those with low abundance, when single chain antibody has low affinity for the antigen.

In another aspect, the present invention provides an engineered antibody, comprising: (i) a first polypeptide comprises a second light chain variable domain (VL2) that binds a second target and a first heavy chain variable domain (VH1) that binds a first target, wherein the VL2 is covalently linked to the VH1; (ii) a second polypeptide comprises a first light chain variable domain (VL1) that binds the first target, a second heavy chain variable domain (VH2) that binds the second target, a hinge domain, and a CH2-CH3 domain of IgG, wherein the VL1 is covalently linked to of the VH2; (iii) a third polypeptide comprises a third heavy chain variable domain (VH3) that bind a third target, a CH1domain, a cysteine-containing hinge domain, and a CH2-CH3 domain of IgG; and (iv) a fourth polypeptide comprises a fourth light chain variable domain (VL3) that binds the third target, and a cysteine-containing CL domain; wherein said VL1 and VH1 associate to form a domain capable of binding said the target; wherein the VL2 and VH2 associate to form a domain capable of binding the second target; wherein the VL3 and VH3 associate to form a domain capable of binding the third target; wherein the VL2 and the VH2 are covalently linked via a disulfide bond; wherein the VL2 and the VH2 independently comprises one or more substitutions that introduce charged amino acids that are electrostatically unfavorable to homodimer formation; wherein the CH1 and the CL are covalently linked via a disulfide bond; and wherein the second and third polypeptide chain are covalently linked via the hinge domains and the CH3 domains.

In some embodiments, the C-terminus of the VL2 is covalently linked to N-terminus of the VH1 and C-terminus of the VL1 is covalently linked to N-terminus of the VH2.

In some embodiments, the N-terminus of the VL2 is covalently linked to C-terminus of the VH1 and N-terminus of the VL1 is covalently linked to C-terminus of the VH2.

In some embodiments, the first polypeptide is a first light chain variable domain (VL1) that binds the first antigen and the second polypeptide is a second heavy chain variable domain (VH2) that binds the second antigen, wherein the VL1 and the VH2 is linked via a first peptide linker.

In some embodiments, the first polypeptide is linked via hinge region of its C terminus to the N terminus of a Fc region. The hinge region comprises a hinge from IgG1, IgG2, IgG3, IgG4 or IgA. The Fc region comprises CH2 and CH3 of IgG1, IgG2, IgG3, IgG4 or IgA.

In some embodiments, the third target and the first target are the same target.

In some embodiments, the third target and the second target are the same target. In some embodiments, the first target and the second target are the same target.

In some embodiments, the CH2-CH3 domain of the second polypeptide and the CH2-CH3 domain of the third polypeptide are different.

In some embodiments, the second polypeptide and the third polypeptide are engineered through modification to CH3 domain interface with different mutations on each domain.

In some embodiments, one of the CH3 domains comprises a replacement of Thr366 with Trp, and the other CH3 domain comprises a replacement of Thr366, Leu368, Tyr407 with Ser, Ala and Val, respectively.

In some embodiments, one of the CH3 domains comprises a replacement of Asp399 and Glu356 with Lys, and the other CH3 domain comprises a replacement of Lys392 and Lys409 with Asp.

In some embodiments, one of the CH3 domains comprises a replacement of Glu356, Glu357 and Asp399 with Lys, and the other CH3 domain comprises a replacement of Lys370, Lys409 and Lys439 with Glu, Asp and Glu, respectively.

In some embodiments, one of the CH3 domains comprises a replacement of Ser364 and Phe405 with His and Ala respectively, and the other CH3 domain comprises a replacement of Tyr349 and Thr394 with Thr and Phe, respectively.

In some embodiments, one of the CH3 domains comprises a replacement of Lys370 and Lys409 with Asp, and the other CH3 domain comprises a replacement of Glu357 and Asp399 with Lys.

In some embodiments, one of the CH3 domains comprises a replacement of Leu351 and Leu368 with Asp and Glu respectively, and the other CH3 domain comprises a replacement of Leu361 and Thr366 with Lys.

### Knob-into-Hole Structure

In some embodiments, the third polypeptide and the fourth polypeptide are covalently linked via a hinge region and form a knob-into-hole structure.

A knob-into-hole structure, also known as “protuberance-into-cavity” strategy which serves to engineer an interface between a first and second polypeptide for hetero-oligomerization.

In general, the preferred interface comprises at least a part of the CH3 domain of an antibody constant domain. “Protuberances” are constructed by replacing small amino acid side chains from the interface of the first polypeptide with larger side chains (e.g. tyrosine or tryptophan). Compensatory “cavities” of identical or similar size to the protuberances are optionally created on the interface of the second polypeptide by replacing large amino acid side chains with smaller ones (e.g. alanine or threonine). Where a suitably positioned and dimensioned protuberance or cavity exists at the interface of either the first or second polypeptide, it is only necessary to engineer a corresponding cavity or protuberance, respectively, at the adjacent interface. See U.S. Pat. No. 8,216,805, the disclosure of which is incorporated by reference in its entirety.

In some embodiments, the fourth polypeptide comprises a hole structure formed by substitutions Y407V, T366S, L368A, and Y349C.

### C. Disease Specific Target

In general, the one of the targets (e.g., first target) is a disease specific target.

By “target” herein is meant an antigen, such as a tumor antigen or cell specific biomarker (such as a protein), or an epitope of an antigen.

The disease specific target could be a tumor target (e.g Her2, Jamnani, F. R., et al. T cells expressing VHH-directed oligoclonal chimeric HER2 antigen receptors: towards tumor-directed oligoclonal T cell therapy. Biochimica et biophysica acta 1840, 378-386 (2014), Even-Desrumeaux, K., Fourquet, P., Secq, V., Baty, D. & Chames, P. Single-domain antibodies: a versatile and rich source of binders for breast cancer diagnostic approaches. Molecular bioSystems 8, 2385-2394 (2012)), neo-antigen (e.g. TRK (Patent publication U.S. Pat. No. 7,750,122 B2)), or disease-specific receptors (e.g. EGFR, see Patent WO2010037838 and Bell, A., et al. Differential tumor-targeting abilities of three single-domain antibody formats. Cancer letters 289, 81-90 (2010)).

In some embodiments, the disease specific target is selected from one of the disease markers, cytokines, or chemokines provided in Table 2.

TABLE 2
Target List

| Receptors | Cytokines | Chemokines | Disease Markers |
|---|---|---|---|
| PDL1 | IL-4 | CCL1 | HER2 |
| PDL2 | IL-16 | CCL2 | HER3 |
| CTLA4 | TGF beta | CCL3 | CEA |
| KIR | IL-1 | CCL4 | Muc-1 |
| IDO-1 | IL-6 | CCL5 | GPCR3 |
| 4-1BB | IL-10 | CCL6 | Alpha fetoprotein (AFP) |
| OX40L | IL-12 | CCL7 | CA15-3 |
| LAG3 | IL-18 | CCL8 | CA27-29 |
| CD47 | IL-17 | CCL9 | CA19-9 |
| CD80 | IL-15 | CCL10 | CA-125 |
| CD86 | IL13 | CCL11 | Calcitonin |
| B7RP1 | IL-23 | CCL12 | Calretinin |
| B7-H3 | IL21 | CCL13 | Carcinoembryonic antigen |
| HVEM | IL-32 | CCL14 | CD34 |
| CD137L | IL-9 | CCL15 | CD99MIC 2 |
| CD70 | IL28 | CCL16 | CD117 |
| GAL9 | Leptin | CCL17 | Chromogranin |
| CD4 | IL9 | CCL18 | TRK |
| TIM3 | IFN | CCL19 | Cytokeratin (various types: TPA, TPS, |
| | | | Cyfra21-1) |
| TIM4 | BAFF | CCL20 | Desmin |
| Adenosine | Oncostatin | CCL21 | Epithelial membrane antigen (EMA) |
| receptor | | | |
| TAM | VEGF | CCL22 | Factor VIII, CD31 FL1 |
| Vista | | CCL23 | Glial fibrillary acidic protein (GFAP) |
| BTLA | Type I | CCL24 | Gross cystic disease fluid protein (GCDFP- |
| | IFNs | | 15) |
| HLA-G | TNF | CCL25 | HMB-45 |
| IDO-2 | RANKL | CCL26 | Human chorionic gonadotropin (hCG) |
| ARG1 | NGF | CCL27 | immunoglobulin |
| GCP3 | CSF | CCL28 | inhibin |
| Trop-2 | TNF-alpha | CXCL1 | keratin (various types) |
| Claudin | CD30L | CXCL2 | lymphocyte marker (various types |
| FOXO | CD40L | CXCL3 | MART-1 (Melan-A) |
| BCMA | CD27L | CXCL4 | Myo D1 |
| TRK | TNFSF10 | CXCL5 | muscle-specific actin (MSA) |
| Her2 | IL-2 | CXCL6 | neurofilament |
| Her3 | BMP | CXCL7 | neuron-specific enolase (NSE) |
| EGFR | GDF | CXCL8 | placental alkaline phosphatase (PLAP) |
| GITR | GDNF | CXCL9 | prostate-specific antigen (PSA) |
| PD1 | | CXCL10 | PTPRC (CD45) |
| CD3 | | CXCL11 | S100 protein |
| CD8 | | CXCL12 | smooth muscle actin (SMA) |
| CD16 | | CXCL13 | synaptophysin |
| CD19 | | CXCL14 | thymidine kinase |
| CD20 | | CXCL15 | thyroglobulin (Tg) |
| CD21 | | CXCL16 | thyroid transcription factor-1 (TTF-1) |
| CD22 | | CXCL17 | Tumor M2-PK |
| CD23 | | FAM19 | vimentin |
| CD24 | | | CA-125 |
| CD27 | | | |
| CD33 | | | Epithelial tumor antigen (ETA) |
| CD38 | | | Tyrosinase |
| CD40 | | | Melanoma-associated antigen (MAGE) |
| CD32 | | | abnormal products of ras, p53 |
| CD64 | | | |
| CD123 | | | |
| CCR1 | | | |
| CCR2 | | | |
| CCR3 | | | |
| CCR4 | | | |
| CCR5 | | | |
| CCR6 | | | |
| CCR7 | | | |
| CCR8 | | | |
| CXCR1 | | | |
| CXCR2 | | | |
| CXCR3 | | | |
| CXCR4 | | | |
| CXCR5 | | | |
| CXCR6 | | | |
| CXCR7 | | | |
| CD116/GM-CSFR | | | |
| CD131/CSFR2B/JL3RB/IL5RB | | | |
| CD115/MCSF R/CSF1R | | | |
| CD114/G-CSFR | | | |
| BMP receptor | | | |
| GDNF receptor | | | |
| TGF-beta recepor | | | |
| FcRn | | | |
| DR | | | |
| IL6R | | | |
| IL | | | |
| GPCR | | | |
| MUC1 | | | |
| prostate stem cell antigen | | | |
| prostate membrane antigen | | | |
| Mesothelin | | | |
| CD47 | | | |
| FGFR1 | | | |
| KLB | | | |

In certain specific embodiments, a target is a tumor marker. In some embodiments, a tumor marker is an antigen that is present in a tumor that is not present in normal organs, tissues, and/or cells. In some embodiments, a tumor marker is an antigen that is more prevalent in a tumor than in normal organs, tissues, and/or cells. In some embodiments, a tumor marker is an antigen that is more prevalent in malignant cancer cells than in normal cells.

By “tumor antigen” herein is meant an antigenic substance produced in tumor cells, i.e., it triggers an immune response in the host. Normal proteins in the body are not antigenic because of self-tolerance, a process in which self-reacting cytotoxic T lymphocytes (CTLs) and autoantibody-producing B lymphocytes are culled “centrally” in primary lymphatic tissue (BM) and “peripherally” in secondary lymphatic tissue (mostly thymus for T-cells and spleen/lymph nodes for B cells). Thus, any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

In some embodiments, a target is preferentially expressed in tumor tissues and/or cells versus normal tissues and/or cells.

In some embodiments of the invention a marker is a tumor marker. The marker may be a polypeptide that is expressed at higher levels on dividing than on non-dividing cells. For example, Her-2/neu (also known as ErbB-2) is a member of the EGF receptor family and is expressed on the cell surface of tumors associated with breast cancer. Another example is a peptide known as F3 that is a suitable targeting agent for directing a nanoparticle to nucleolin (Porkka et al., 2002, Proc. Natl. Acad. Sci., USA, 99:7444; and Christian et al., 2003, J. Cell Biol., 163:871). It has been shown that targeted particles comprising a nanoparticle and the A10 aptamer (which specifically binds to PSMA) were able to specifically and effectively deliver docetaxel to prostate cancer tumors.

Antibodies or other drug that specifically target these tumor targets specifically interfere with and regulate signaling pathways of the biological behavior of tumor cells regulate directly, or block signaling pathway to inhibit tumor cell growth or induce apoptosis. To date, there are dozens of target drugs have been approved for solid tumors or hematological malignancies clinical research and treatment, and there are number of targeted drugs for hematological malignancies.

In some embodiments, the tumor antigen (or tumor target) is selected from the group consisting of: CD2, CD19, CD20, CD22, CD27, CD33, CD37, CD38, CD40, CD44, CD47, CD52, CD56, CD70, CD79, and CD137.

In some embodiments, the tumor antigen (or tumor target) is selected from the group consisting of: 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin [symbol] Integrin [symbol] , KIR, LAG-3, Lewis Y antigen, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof. The variants of the tumor antigen encompass various mutants or polympormisms known in the art and/or naturally occurred.

In some embodiments, the disease specific target is selected from antigens that are overexpressed in cancer cells, including intercellular adhesion molecule 1 (ICAM-1), ephrin type-A receptor 2 (EphA2), ephrin type-A receptor 3 (EphA3), ephrin type-A receptor 4 (EphA4), or activated leukocyte cell adhesion molecule (ALCAM).

In some embodiments, the disease specific target is selected from cancer- or tumor-associated guide antigens, include CD30, CD33, PSMA, mesothelin, CD44, CD73, CD38, Mucin 1 cell surface associated (MUC1), Mucin 2 oligomeric mucus gel-forming (MUC2), and MUC16 (CA-125).

In some embodiments, the disease specific target is selected from CD30, CD33, carcinoembroyonic antigen (CEA), mesothelin, cathepsin G, CD44, CD73, CD38, Muc1, Muc2, Muc16, preferentially expressed antigen of melanoma (PRAME), CD52, EpCAM, CEA, gpA33, Mucins, tumor associated glycoprotein 72 (TAG-72), carbonic anhydrase IX, PSMA, folate binding protein, gangliosides, Lewis-Y, immature laminin receptor, BING-4, calcium-activated chloride channel 2 (CaCC), gp100, synovial sarcoma X breakpoint 2 (SSX-2), or SAP-I.

In some embodiments, the disease specific target is selected from CD30, CD33, arcinoembroyonic antigen (CEA), mesothelin, cathepsin G, CD44, CD73, CD38, Muc1, Muc16, preferentially expressed antigen of melanoma (PRAME), CD52, EpCAM, CEA, gpA33, Mucins, TAG-72, carbonic anhydrase IX, PSMA, folate binding protein, gangliosides or Lewis-Y, ICAM-1, EphA2, or ALCAM.

### D. Immune Regulatory Function Target

In general, one of the antigen (e.g. the second antigen) is an immune regulatory function target that is related to the disease target.

The immune regulatory function target could be a checkpoint receptor (e.g. PD-L1 (patent WO2017020801-PAMPH-866 and Zhang, F., et al. Structural basis of a novel PD-L1 nanobody for immune checkpoint blockade. Cell discovery 3, 17004 (2017)), or a regulatory cytokines receptor, etc.

In some embodiments, the immune regulatory function target is selected from one of the receptors provided in Table 2.

In some embodiments, the immune regulatory function target is related to NK cell activating or inhibiting pathway, and is selected from CD16, CD38, NKG2D, NKG2A, NKp46 or Killer-cell immunoglobulin like receptors (KIRs).

In some embodiments, the immune regulatory function target is related to checkpoint inhibitory pathway (which can be active in T cell), and is selected from PD1, CTLA4, and Tim3.

The single domain of the present invention binds specifically to a target.

By “target” or “marker” herein is meant any entity that is capable of specifically binding to a particular targeted therapeutic, such as Her2/Neu. In some embodiments, targets are specifically associated with one or more particular cell or tissue types. In some embodiments, targets are specifically associated with one or more particular disease states. In some embodiments, targets are specifically associated with one or more particular developmental stages. For example, a cell type specific marker is typically expressed at levels at least 2-fold greater in that cell type than in a reference population of cells. In some embodiments, the cell type specific marker is present at levels at least 3-fold, at least 4-fold, at least 5-fold, at least 6-fold, at least 7-fold, at least 8-fold, at least 9-fold, at least 10-fold, at least 50-fold, at least 100-fold, or at least 1,000-fold greater than its average expression in a reference population. Detection or measurement of a cell type specific marker may make it possible to distinguish the cell type or types of interest from cells of many, most, or all other types. In some embodiments, a target can comprise a protein, a carbohydrate, a lipid, and/or a nucleic acid, as described herein.

By “specifically binds” or “preferably binds” herein is meant that the binding between two binding partners (e.g., between a targeting moiety and its binding partner) is selective for the two binding partners and can be discriminated from unwanted or non-specific interactions. For example, the ability of an antigen-binding moiety to bind to a specific antigenic determinant can be measured either through an enzyme-linked immunosorbent assay (ELISA) or other techniques familiar to one of skill in the art, e.g. surface plasmon resonance technique (analyzed on a BIAcore instrument) (Liljeblad et al., Glyco J 17, 323-329 (2000)), and traditional binding assays (Heeley, Endocr Res 28, 217-229 (2002)). The terms “anti-[antigen] antibody” and “an antibody that binds to [antigen]” refer to an antibody that is capable of binding the respective antigen with sufficient affinity such that the antibody is useful as a diagnostic and/or therapeutic agent in targeting the antigen. In some embodiments, the extent of binding of an anti-[antigen] antibody to an unrelated protein is less than about 10% of the binding of the antibody to the antigen as measured, e.g., by a radioimmunoassay (RIA).

In some embodiments, the antigen binding that binds to antigen has a dissociation constant (KD) of <100 μM, <10 μM, <1 μM, <100 nM, <10 nM, <1 nM, <0.1 nM, <0.01 nM, or <0.001 nM (e.g. 10⁻⁴ M or less, e.g. from 10⁻⁴ M to 10⁻¹² M, e.g., from 10⁻⁹ M to 10⁻¹³ M), and preferably from 10⁻⁴ M to 10⁻⁶ M.

### E. Antibodies and Functional Fragments

In some embodiments, the targeted therapeutic comprises an antibody, or a functional fragment thereof.

By immunoglobulin” or “antibody” herein is meant a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. An antibody or antibody fragment may be conjugated or otherwise derivatized within the scope of the claimed subject matter. Such antibodies include IgG1, IgG2a, IgG3, IgG4 (and IgG4 subforms), as well as IgA isotypes.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity and comprise an Fc region or a region equivalent to the Fc region of an immunoglobulin The terms “full-length antibody”, “intact antibody”, “and “whole antibody” are used herein interchangeably to refer to an antibody having a structure substantially similar to a native antibody structure or having heavy chains that contain an Fc region as defined herein.

By “native antibodies” herein is meant naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CHI, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The light chain of an antibody may be assigned to one of two types, called kappa (κ) and lambda (λ), based on the amino acid sequence of its constant domain.

By “antibody fragment” herein is meant a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), single-domain antibodies, and multispecific antibodies formed from antibody fragments. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B 1). Antibody fragments can be made by various techniques, including but not limited to proteolytic digestion of an intact antibody as well as production by recombinant host cells (e.g. E. coli or phage), as described herein.

By “antigen binding domain” herein is meant the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

By “variable region” or “variable domain” herein is meant the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

By “hypervariable region” or “HVR” herein is meant each of the regions of an antibody variable domain which are hypervariable in sequence and/or form structurally defined loops ““hypervariable loops”). Generally, native four-chain antibodies comprise six HVRs; three in the VH (HI, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The antibody of the present invention can be chimeric antibodies, humanized antibodies, human antibodies, or antibody fusion proteins.

By “chimeric antibody” herein is meant a recombinant protein that contains the variable domains of both the heavy and light antibody chains, including the complementarity determining regions (CDRs) of an antibody derived from one species, preferably a rodent antibody, more preferably a murine antibody, while the constant domains of the antibody molecule are derived from those of a human antibody. For veterinary applications, the constant domains of the chimeric antibody may be derived from that of other species, such as a subhuman primate, cat or dog.

By “humanized antibody” herein is meant a recombinant protein in which the CDRs from an antibody from one species; e.g., a rodent antibody, are transferred from the heavy and light variable chains of the rodent antibody into human heavy and light variable domains. The constant domains of the antibody molecule are derived from those of a human antibody. In some embodiments, specific residues of the framework region of the humanized antibody, particularly those that are touching or close to the CDR sequences, may be modified, for example replaced with the corresponding residues from the original rodent, subhuman primate, or other antibody.

By “human antibody” herein is meant an antibody obtained, for example, from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al, Nature Genet. 7: 13 (1994), Lonberg et al, Nature 368:856 (1994), and Taylor et al, Int. Immun. 6:579 (1994). A fully human antibody also can be constructed by genetic or chromosomal transfection methods, as well as phage display technology, all of which are known in the art. See for example, McCafferty et al, Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g. Johnson and Chiswell, Current Opinion in Structural Biology 3:5564-571 (1993). Human antibodies may also be generated by in vitro activated B cells. See U.S. Pat. Nos. 5,567,610 and 5,229,275, which are incorporated herein by reference in their entirety.

By “antibody fusion protein” herein is meant a recombinantly-produced antigen-binding molecule in which two or more of the same or different natural antibody, single-chain antibody or antibody fragment segments with the same or different specificities are linked. A fusion protein comprises at least one specific binding site. Valency of the fusion protein indicates the total number of binding arms or sites the fusion protein has to antigen(s) or epitope(s); i.e., monovalent, bivalent, trivalent or mutlivalent. The multivalency of the antibody fusion protein means that it can take advantage of multiple interactions in binding to an antigen, thus increasing the avidity of binding to the antigen, or to different antigens. Specificity indicates how many different types of antigen or epitope an antibody fusion protein is able to bind; i.e., monospecific, bispecific, trispecific, multispecific. Using these definitions, a natural antibody, e.g., an IgG, is bivalent because it has two binding arms but is monospecific because it binds to one type of antigen or epitope. A monospecific, multivalent fusion protein has more than one binding site for the same antigen or epitope. For example, a monospecific diabody is a fusion protein with two binding sites reactive with the same antigen. The fusion protein may comprise a multivalent or multispecific combination of different antibody components or multiple copies of the same antibody component. The fusion protein may additionally comprise a therapeutic agent.

By “target” or “marker” herein is meant any entity that is capable of specifically binding to a particular targeting moiety. In some embodiments, targets are specifically associated with one or more particular cell or tissue types. In some embodiments, targets are specifically associated with one or more particular disease states. In some embodiments, targets are specifically associated with one or more particular developmental stages. For example, a cell type specific marker is typically expressed at levels at least 2 fold greater in that cell type than in a reference population of cells. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1,000 fold greater than its average expression in a reference population. Detection or measurement of a cell type specific marker may make it possible to distinguish the cell type or types of interest from cells of many, most, or all other types. In some embodiments, a target can comprise a protein, a carbohydrate, a lipid, and/or a nucleic acid, as described herein.

A substance is considered to be “targeted” for the purposes described herein if it specifically binds to a nucleic acid targeting moiety. In some embodiments, a nucleic acid targeting moiety specifically binds to a target under stringent conditions. An inventive complex or compound comprising targeting moiety is considered to be “targeted” if the targeting moiety specifically binds to a target, thereby delivering the entire complex or compound composition to a specific organ, tissue, cell, extracellular matrix component, and/or intracellular compartment.

In certain embodiments, antibody in accordance with the present invention comprise a single domain antibody or fragment which specifically binds to one or more targets (e.g. antigens) associated with an organ, tissue, cell, extracellular matrix component, and/or intracellular compartment. In some embodiments, compounds comprise a targeting moiety which specifically binds to targets associated with a particular organ or organ system. In some embodiments, compounds in accordance with the present invention comprise a nuclei targeting moiety which specifically binds to one or more intracellular targets (e.g. organelle, intracellular protein). In some embodiments, compounds comprise a targeting moiety which specifically binds to targets associated with diseased organs, tissues, cells, extracellular matrix components, and/or intracellular compartments. In some embodiments, compounds comprise a targeting moiety which specifically binds to targets associated with particular cell types (e.g. endothelial cells, cancer cells, malignant cells, prostate cancer cells, etc.).

In some embodiments, antibodys in accordance with the present invention comprise a domain antibody or fragment which binds to a target that is specific for one or more particular tissue types (e.g. liver tissue vs. prostate tissue). In some embodiments, compounds in accordance with the present invention comprise a domain which binds to a target that is specific for one or more particular cell types (e.g. T cells vs. B cells). In some embodiments, antibodies in accordance with the present invention comprise a domain which binds to a target that is specific for one or more particular disease states (e.g. tumor cells vs. healthy cells). In some embodiments, compounds in accordance with the present invention comprise a targeting moiety which binds to a target that is specific for one or more particular developmental stages (e.g. stem cells vs. differentiated cells).

In some embodiments, a target may be a marker that is exclusively or primarily associated with one or a few cell types, with one or a few diseases, and/or with one or a few developmental stages. A cell type specific marker is typically expressed at levels at least 2 fold greater in that cell type than in a reference population of cells which may consist, for example, of a mixture containing cells from a plurality (e.g., 5-10 or more) of different tissues or organs in approximately equal amounts. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1000 fold greater than its average expression in a reference population. Detection or measurement of a cell type specific marker may make it possible to distinguish the cell type or types of interest from cells of many, most, or all other types.

In some embodiments, a target comprises a protein, a carbohydrate, a lipid, and/or a nucleic acid. In some embodiments, a target comprises a protein and/or characteristic portion thereof, such as a tumor-marker, integrin, cell surface receptor, transmembrane protein, intercellular protein, ion channel, membrane transporter protein, enzyme, antibody, chimeric protein, glycoprotein, etc. In some embodiments, a target comprises a carbohydrate and/or characteristic portion thereof, such as a glycoprotein, sugar (e.g., monosaccharide, disaccharide, polysaccharide), glycocalyx (i.e., the carbohydrate-rich peripheral zone on the outside surface of most eukaryotic cells) etc. In some embodiments, a target comprises a lipid and/or characteristic portion thereof, such as an oil, fatty acid, glyceride, hormone, steroid (e.g., cholesterol, bile acid), vitamin (e.g. vitamin E), phospholipid, sphingolipid, lipoprotein, etc. In some embodiments, a target comprises a nucleic acid and/or characteristic portion thereof, such as a DNA nucleic acid; RNA nucleic acid; modified DNA nucleic acid; modified RNA nucleic acid; nucleic acid that includes any combination of DNA, RNA, modified DNA, and modified RNA.

Numerous markers are known in the art. Typical markers include cell surface proteins, e.g., receptors. Exemplary receptors include, but are not limited to, the transferrin receptor; LDL receptor; growth factor receptors such as epidermal growth factor receptor family members (e.g., EGFR, Her2, Her3, Her4) or vascular endothelial growth factor receptors, cytokine receptors, cell adhesion molecules, integrins, selectins, and CD molecules. The marker can be a molecule that is present exclusively or in higher amounts on a malignant cell, e.g., a tumor antigen.

In some embodiments, the binding domain binds to a tumor cell specifically or preferably in comparison to a non-tumor cell.

The binding of target moiety to tumor cell can be measured using assays known in the art.

In some embodiments, the tumor cell is of a carcinoma, a sarcoma, a lymphoma, a myeloma, or a central nervous system cancer.

In some embodiments, the binding domain is capable of binding to a tumor antigen specifically or preferably in comparison to a non-tumor antigen.

In certain specific embodiments, a target is a tumor marker. In some embodiments, a tumor marker is an antigen that is present in a tumor that is not present in normal organs, tissues, and/or cells. In some embodiments, a tumor marker is an antigen that is more prevalent in a tumor than in normal organs, tissues, and/or cells. In some embodiments, a tumor marker is an antigen that is more prevalent in malignant cancer cells than in normal cells.

In some embodiments, the targeting moiety comprises folic acid or a derivative thereof.

In recent years, research on folic acid had made great progress. Folic acid is a small molecule vitamin that is necessary for cell division. Tumor cells divide abnormally and there is a high expression of folate receptor (FR) on tumor cell surface to capture enough folic acid to support cell division.

Data indicate FR expression in tumor cells is 20-200 times higher than normal cells. The expression rate of FR in various malignant tumors are: 82% in ovarian cancer, 66% in non-small cell lung cancer, 64% in kidney cancer, 34% in colon cancer, and 29% in breast cancer (Xia W, Low P S. Late-targeted therapies for cancer. J Med Chem. 2010; 14; 53 (19):6811-24). The expression rate of FA and the degree of malignancy of epithelial tumor invasion and metastasis is positively correlated. FA enters cell through FR mediated endocytosis, and FA through its carboxyl group forms FA complexes with drugs which enter the cells. Under acidic conditions (pH value of 5), FR separates from the FA, and FA releases drugs into the cytoplasm.

Clinically, the system can be used to deliver drugs selectively attack the tumor cells. Folic acid has small molecular weight, has non-immunogenicity and high stability, and is inexpensive to synthesis. More importantly, chemical coupling between the drug and the carrier is simple, and as such using FA as targeting molecule to construct drug delivery system has become a research hotspot for cancer treatment. Currently EC145 (FA chemotherapy drug conjugate compound) that is in clinical trials can effectively attack cancer cells (Pribble P and Edelman M J. EC145: a novel targeted agent for adenocarcinoma of the lung. Expert Opin. Investig. Drugs (2012) 21:755-761).

In some embodiments, the targeting moiety comprises extracellular domains (ECD) or soluble form of PD-1, PDL-1, CTLA4, CD47, BTLA, KIR, TIM3, 4-1BB, and LAG3, full length of partial of a surface ligand Amphiregulin, Betacellulin, EGF, Ephrin, Epigen, Epiregulin, IGF, Neuregulin, TGF, TRAIL, or VEGF.

In some embodiments, the targeting moiety comprises a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, or an antibody analogue comprising one or more CDRs.

In some embodiments, the targeting moiety is an antibody, or antibody fragment, that is selected based on its specificity for an antigen expressed on a target cell, or at a target site, of interest. A wide variety of tumor-specific or other disease-specific antigens have been identified and antibodies to those antigens have been used or proposed for use in the treatment of such tumors or other diseases. The antibodies that are known in the art can be used in the compounds of the invention, in particular for the treatment of the disease with which the target antigen is associated. Examples of target antigens (and their associated diseases) to which an antibody-linker-drug conjugate of the invention can be targeted include: CD2, CD19, CD20, CD22, CD27, CD33, CD37, CD38, CD40, CD44, CD47, CD52, CD56, CD70, CD79, CD137, 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin [symbol] F Integrin [symbol] , KIR, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3.

### F. Manufacturing the Antibodies

All of the antibody formats are based on heavy chain and light chain of an IgG antibody that can be manufactured using methods known in the art, which typically include steps of construction of expression cassette for the heavy and light chain genes, co-transfect the two genes into a suitable cell system to produce the recombinant antibody and to make a stable and high-productive cell clone, cell fermentation to produce cGMP final antibody product.

### III. PHARMACEUTICAL FORMULATIONS AND ADMINISTRATION

The present invention further relates to a pharmaceutical formulation comprising a compound of the invention or a pharmaceutically acceptable salt thereof, and one or more pharmaceutically acceptable carriers.

The compounds described herein including pharmaceutically acceptable carriers such as addition salts or hydrates thereof, can be delivered to a patient using a wide variety of routes or modes of administration. Suitable routes of administration include, but inhalation, transdermal, oral, rectal, transmucosal, intestinal and parenteral administration, including intramuscular, subcutaneous and intravenous injections. Preferably, the compounds of the invention comprising an antibody or antibody fragment as the targeting moiety are administered parenterally, more preferably intravenously.

As used herein, the terms “administering” or “administration” are intended to encompass all means for directly and indirectly delivering a compound to its intended site of action.

The compounds described herein, or pharmaceutically acceptable salts and/or hydrates thereof, may be administered singly, in combination with other compounds of the invention, and/or in cocktails combined with other therapeutic agents. Of course, the choice of therapeutic agents that can be co-administered with the compounds of the invention will depend, in part, on the condition being treated.

For example, when administered to patients suffering from a disease state caused by an organism that relies on an autoinducer, the compounds of the invention can be administered in cocktails containing agents used to treat the pain, infection and other symptoms and side effects commonly associated with the disease. Such agents include, e.g., analgesics, antibiotics, etc.

When administered to a patient undergoing cancer treatment, the compounds may be administered in cocktails containing anti-cancer agents and/or supplementary potentiating agents. The compounds may also be administered in cocktails containing agents that treat the side-effects of radiation therapy, such as anti-emetics, radiation protectants, etc.

Supplementary potentiating agents that can be co-administered with the compounds of the invention include, e.g., tricyclic anti-depressant drugs (e.g., imipramine, desipramine, amitriptyline, clomipramine, trimipramine, doxepin, nortriptyline, protriptyline, amoxapine and maprotiline); non-tricyclic and anti-depressant drugs (e.g., sertraline, trazodone and citalopram); Ca+2 antagonists (e.g., verapamil, nifedipine, nitrendipine and caroverine); amphotericin; triparanol analogues (e.g., tamoxifen); antiarrhythmic drugs (e.g., quinidine); antihypertensive drugs (e.g., reserpine); thiol depleters (e.g., buthionine and sulfoximine); and calcium leucovorin.

The active compound(s) of the invention are administered per se or in the form of a pharmaceutical composition wherein the active compound(s) is in admixture with one or more pharmaceutically acceptable carriers, excipients or diluents. Pharmaceutical compositions for use in accordance with the present invention are typically formulated in a conventional manner using one or more physiologically acceptable carriers comprising excipients and auxiliaries, which facilitate processing of the active compounds into preparations which, can be used pharmaceutically. Proper formulation is dependent upon the route of administration chosen.

For transmucosal administration, penetrants appropriate to the barrier to be permeated are used in the formulation. Such penetrants are generally known in the art.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, and suspensions for oral ingestion by a patient to be treated. Pharmaceutical preparations for oral use can be obtained solid excipient, optionally grinding a resulting mixture, and processing the mixture of granules, after adding suitable auxiliaries, if desired to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Injection is a preferred method of administration for the compositions of the current invention. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents may be added, such as the cross-linked polyvinyl pyrrolidone, agar, or alginic acid or a salt thereof such as sodium alginate.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions. For injection, the agents of the invention may be formulated in aqueous solutions, preferably in physiologically compatible buffers such as Hanks's solution, Ringer's solution, or physiological saline buffer.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include calcium carbonate, calcium phosate, various sugars, starches, cellulose derivatives, gelatin, and polymers such as polyethylene glycols.

A preferred pharmaceutical composition is a composition formulated for injection such as intravenous injection and includes about 0.01% to about 100% by weight of the compound of the present invention, based upon 100% weight of total pharmaceutical composition. The drug-ligand conjugate may be an antibody-cytotoxin conjugate where the antibody has been selected to target a particular cancer.

In some embodiments, the pharmaceutical composition of the present invention further comprises an additional therapeutic agent.

In some embodiments, the additional therapeutic agent is an anticancer agent.

In some embodiments, the additional anticancer agent is selected from an antimetabolite, an inhibitor of topoisomerase I and II, an alkylating agent, a microtubule inhibitor, an antiandrogen agent, a GNRh modulator or mixtures thereof.

In some embodiments, the additional therapeutic agent is a chemotherapeutic agent.

By “chemotherapeutic agent” herein is meant a chemical compound useful in the treatment of cancer. Examples are but not limited to: Gemcitabine, Irinotecan, Doxorubicin, 5-Fluorouracil, Cytosine arabinoside (“Ara-C”), Cyclophosphamide, Thiotepa, Busulfan, Cytoxin, TAXOL, Methotrexate, Cisplatin, Melphalan, Vinblastine and Carboplatin.

In some embodiments, the second chemotherapeutic agent is selected from the group consisting of tamoxifen, raloxifene, anastrozole, exemestane, letrozole, imatanib, paclitaxel, cyclophosphamide, lovastatin, minosine, gemcitabine, cytarabine, 5-fluorouracil, methotrexate, docetaxel, goserelin, vincristine, vinblastine, nocodazole, teniposide etoposide, gemcitabine, epothilone, vinorelbine, camptothecin, daunorubicin, actinomycin D, mitoxantrone, acridine, doxorubicin, epirubicin, or idarubicin.

### IV. KITS

In another aspect, the present invention provides kits containing the therapeutic combinations provided herein and directions for using the therapeutic combinations. The kit may also include a container and optionally one or more vial, test tube, flask, bottle, or syringe. Other formats for kits will be apparent to those of skill in the art and are within the scope of the present invention.

### V. MEDICAL USE

In another aspect, the present invention provides a method for treating a disease condition in a subject that is in need of such treatment, comprising: administering to the subject a therapeteutic combination or pharmaceutical composition comprising a therapeutically effective amount of the compound of the present invention or a pharmaceutically acceptable salt thereof, and a pharmaceutical acceptable carrier.

In addition to the compositions and constructs described above, the present invention also provides a number of uses of the combinations of the invention. Uses of the combinations of the current invention include: killing or inhibiting the growth, proliferation or replication of a tumor cell or cancer cell, treating cancer, treating a pre-cancerous condition, preventing the multiplication of a tumor cell or cancer cell, preventing cancer, preventing the multiplication of a cell that expresses an auto-immune antibody. These uses comprise administering to an animal such as a mammal or a human in need thereof an effective amount of a compound of the present invention.

The combination of the current invention is useful for treating diseases such as cancer in a subject, such as a human being. Combinations and uses for treating tumors by providing a subject the composition in a pharmaceutically acceptable manner, with a pharmaceutically effective amount of a composition of the present invention are provided.

By “cancer” herein is meant the pathological condition in humans that is characterized by unregulated cell proliferation. Examples include but are not limited to: carcinoma, lymphoma, blastoma, and leukemia. More particular examples of cancers include but are not limited to: lung (small cell and non-small cell), breast, prostate, carcinoid, bladder, gastric, pancreatic, liver (hepatocellular), hepatoblastoma, colorectal, head and neck squamous cell carcinoma, esophageal, ovarian, cervical, endometrial, mesothelioma, melanoma, sarcoma, osteosarcoma, liposarcoma, thyroid, desmoids, chronic myelocytic leukemia (AML), and chronic myelocytic leukemia (CML).

By “inhibiting” or “treating” or “treatment” herein is meant to reduction, therapeutic treatment and prophylactic or preventative treatment, wherein the objective is to reduce or prevent the aimed pathologic disorder or condition. In one example, following administering of a compound of the present invention, a cancer patient may experience a reduction in tumor size. “Treatment” or “treating” includes (1) inhibiting a disease in a subject experiencing or displaying the pathology or symptoms of the disease, (2) ameliorating a disease in a subject that is experiencing or displaying the pathology or symptoms of the disease, and/or (3) affecting any measurable decrease in a disease in a subject or patient that is experiencing or displaying the pathology or symptoms of the disease. To the extent a compound of the present invention may prevent growth and/or kill cancer cells, it may be cytostatic and/or cytotoxic.

By “therapeutically effective amount” herein is meant an amount of a compound provided herein effective to “treat” a disorder in a subject or mammal. In the case of cancer, the therapeutically effective amount of the drug may either reduce the number of cancer cells, reduce the tumor size, inhibit cancer cell infiltration into peripheral organs, inhibit tumor metastasis, inhibit tumor growth to certain extent, and/or relieve one or more of the symptoms associated with the cancer to some extent.

Administration “in combination with” one or more further therapeutic agents includes simultaneous (concurrent) and consecutive administration in any order. As used herein, the term “pharmaceutical combination” refers to a product obtained from mixing or combining active ingredients, and includes both fixed and non-fixed combinations of the active ingredients. The term “fixed combination” means that the active ingredients and a co-agent are both administered to a patient simultaneously in the form of a single entity or dosage. The term “non-fixed combination” means that the active ingredients and a co-agent are both administered to a patient as separate entities either simultaneously, concurrently or sequentially with no specific time limits, wherein such administration provides therapeutically effective levels of the active ingredients in the body of the patient. The latter also applies to cocktail therapy, e.g. the administration of three or more active ingredients.

In some embodiments, the diseases condition is tumor or cancer. In some embodiments, the cancer or tumor is selected from stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer or lymphoma.

In some embodiments, the disease condition comprises abnormal cell proliferation, such as a pre-cancerous lesion.

The current invention is particularly useful for the treatment of cancer and for the inhibition of the multiplication of a tumor cell or cancer cell in an animal. Cancer, or a precancerous condition, includes a tumor, metastasis, or any disease or disorder characterized by uncontrolled cell growth, can be treated or prevented by administration the drug-ligand complex of the current invention. The compound delivers the activating moiety to a tumor cell or cancer cell. In some embodiments, the targeting moiety specifically binds to or associates with a cancer-cell or a tumor-cell-associated antigen. Because of its close proximity to the ligand, after being internalized, the activating moiety can be taken up inside a tumor cell or cancer cell through, for example, receptor-mediated endocytosis. The antigen can be attached to a tumor cell or cancer cell or can be an extracellular matrix protein associated with the tumor cell or cancer cell. Once inside the cell, the linker is hydrolytically or enzymatically cleaved by a tumor-cell or cancer-cell-associated proteases, thereby releasing the activating moiety. The released activating moiety is then free to diffuse and induce or enhance immune activity of immune cells or tumor cells. In an alternative embodiment, the activating moiety is cleaved from the compound tumor microenvironment, and the drug subsequently penetrates the cell.

Representative examples of precancerous conditions that may be targeted by the compounds of the present invention, include: metaplasia, hyperplysia, dysplasia, colorectal polyps, actinic ketatosis, actinic cheilitis, human papillomaviruses, leukoplakia, lychen planus and Bowen's disease.

Representative examples of cancers or tumors that may be targeted by compounds of the present invention include: lung cancer, colon cancer, prostate cancer, lymphoma, melanoma, breast cancer, ovarian cancer, testicular cancer, CNS cancer, renal cancer, kidney cancer, pancreatic cancer, stomach cancer, oral cancer, nasal cancer, cervical cancer and leukemia. It will be readily apparent to the ordinarily skilled artisan that the particular targeting moiety used in the compound can be chosen such that it targets the activating moiety to the tumor tissue to be treated with the drug (i.e., a targeting agent specific for a tumor-specific antigen is chosen). Examples of such targeting moiety are well known in the art, examples of which include anti-Her2 for treatment of breast cancer, anti-CD20 for treatment of lymphoma, anti-PSMA for treatment of prostate cancer and anti-CD30 for treatment of lymphomas, including non-Hodgkin's lymphoma.

In some embodiments, the abnormal proliferation is of cancer cells.

In some embodiments, the cancer is selected from the group consisting of: breast cancer, colorectal cancer, diffuse large B-cell lymphoma, endometrial cancer, follicular lymphoma, gastric cancer, glioblastoma, head and neck cancer, hepatocellular cancer, lung cancer, melanoma, multiple myeloma, ovarian cancer, pancreatic cancer, prostate cancer, and renal cell carcinoma.

In some embodiments, the present invention provides a compound for use in killing a cell. The compound is administered to the cell in an amount sufficient to kill said cell. In an exemplary embodiment, the compound is administered to a subject bearing the cell. In a further exemplary embodiment, the administration serves to retard or stop the growth of a tumor that includes the cell (e.g., the cell can be a tumor cell). For the administration to retard the growth, the rate of growth of the cell should be at least 10% less than the rate of growth before administration. Preferably, the rate of growth will be retarded at least 20%, 30%, 40%, 50%, 60%, 70%, 80%, 90%, or completely stopped.

Additionally, the present invention provides a compound or a pharmaceutical composition of the present invention for use as a medicament. The present invention also provides a compound or a pharmaceutical composition for killing, inhibiting or delaying proliferation of a tumor or cancer cell.

### Effective Dosages

Pharmaceutical compositions suitable for use with the present invention include compositions wherein the active ingredient is contained in a therapeutically effective amount, i.e., in an amount effective to achieve its intended purpose. The actual amount effective for a particular application will depend, inter alia, on the condition being treated. Determination of an effective amount is well within the capabilities of those skilled in the art, especially in light of the detailed disclosure herein.

For any compound described herein, the therapeutically effective amount can be initially determined from cell culture assays. Target plasma concentrations will be those concentrations of active compound(s) that are capable of inhibition cell growth or division. In preferred embodiments, the cellular activity is at least 25% inhibited. Target plasma concentrations of active compound(s) that are capable of inducing at least about 30%, 50%, 75%, or even 90% or higher inhibition of cellular activity are presently preferred. The percentage of inhibition of cellular activity in the patient can be monitored to assess the appropriateness of the plasma drug concentration achieved, and the dosage can be adjusted upwards or downwards to achieve the desired percentage of inhibition.

As is well known in the art, therapeutically effective amounts for use in humans can also be determined from animal models. For example, a dose for humans can be formulated to achieve a circulating concentration that has been found to be effective in animals. The dosage in humans can be adjusted by monitoring cellular inhibition and adjusting the dosage upwards or downwards, as described above.

A therapeutically effective dose can also be determined from human data for compounds which are known to exhibit similar pharmacological activities. The applied dose can be adjusted based on the relative bioavailability and potency of the administered compound as compared with the known compound.

Adjusting the dose to achieve maximal efficacy in humans based on the methods described above and other methods as are well-known in the art is well within the capabilities of the ordinarily skilled artisan.

In the case of local administration, the systemic circulating concentration of administered compound will not be of particular importance. In such instances, the compound is administered so as to achieve a concentration at the local area effective to achieve the intended result.

Therapeutic amounts of specific antibodies disclosed herein can also be administered, as a component of the combination, with the immunotherperutics, either in a single mixure form, or separately. In some embodiments, therapeutic amounts are amounts which eliminate or reduce the patient's tumor burden, or which prevent or reduce the proliferation of metastatic cells. The dosage will depend on many parameters, including the nature of the tumor, patient history, patient condition, the possible co-use of other oncolytic agents, and methods of administration. Methods of administration include injection (e.g., parenteral, subcutaneous, intravenous, intraperitoneal, etc.) for which the antibodies are provided in a nontoxic pharmaceutically acceptable carrier such as water, saline, Ringer's solution, dextrose solution, 5% human serum albumin, fixed oils, ethyl oleate, or liposomes. Typical dosages may range from about 0.01 to about 20 mg/kg, such as from about 0.1 to about 10 mg/kg. Other effective methods of administration and dosages may be determined by routine experimentation and are within the scope of this invention.

The therapeutically effective amount of the agents (disclosed herein) administered, when it is used for combination therapy, can vary depending upon the desired effects and the subject to be treated. For example, the subject can receive at least 1 mg/kg (such as 1 mg/kg to 20 mg/kg, 2.5 mg/kg to 10 mg/kg, or 3.75 mg/kg to 5 mg/kg) intravenously of each antibody agent. The dosage can be administered in divided doses (such as 2, 3, or 4 divided doses per day), or in a single dosage.

In the method for combined administration, the agent may be simultaneously administered with the antibody used in the present invention, or the agent may be administered before or after the administration of the antibody used in the present invention.

For other modes of administration, dosage amount and interval can be adjusted individually to provide plasma levels of the administered compound effective for the particular clinical indication being treated. For example, in one embodiment, a compound according to the invention can be administered in relatively high concentrations multiple times per day. Alternatively, it may be more desirable to administer a compound of the invention at minimal effective concentrations and to use a less frequent administration regimen. This will provide a therapeutic regimen that is commensurate with the severity of the individual's disease.

Utilizing the teachings provided herein, an effective therapeutic treatment regimen can be planned which does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

[1] Holliger P, Prospero T, Winter G. “Diabodies”: small bivalent and bispecific antibody fragments. Proc Natl Acad Sci USA. 1993 Jul. 15; 90(14):6444-8.
[2] Whitlow M, Filpula D, Rollence M L, Feng S L, Wood J F. Multivalent Fvs: characterization of single-chain Fv oligomers and preparation of a bispecific Fv. Protein Eng. 1994 August; 7(8):1017-26.
[3] Zhu Z, Lewis G D, Carter P. Engineering high affinity humanized anti-p185HER2anti-CD3 bispecific F(ab′)2 for efficient lysis of p185HER2 overexpressing tumor cells. Int J Cancer. 1995 Jul. 28; 62(3):319-24.
[4] Lu D1, Zhang H, Koo H, etc. A fully human recombinant IgG-like bispecific antibody to both the epidermal growth factor receptor and the insulin-like growth factor receptor for enhanced antitumor activity. J Biol Chem. 2005 May 20; 280(20):19665-72.
[5] WO2006020258A2
[6]U.S. Pat. No. 7,838,637
[7] CN1234406A
[8] EP0952218
[9]Müller D1, Karle A, Meissburger B, etc. Improved pharmacokinetics of recombinant bispecific antibody molecules by fusion to human serum albumin. J Biol Chem. 2007 Apr. 27; 282(17):12650-60.
[10] Asano R1, Shimomura I, Konno S, etc. Rearranging the domain order of a diabody-based IgG-like bispecific antibody enhances its antitumor activity and improves its degradation resistance and pharmacokinetics. MAbs. 2014; 6(5):1243-54.
[11] U.S. Pat. No. 9,315,584
[12] Reiter, Y., Brinkmann, R. J., Kreitman, R. J., etc. Stabilization of the Fv Fragments in Recombinant Immunotoxins by Disulfide Bonds Engineered Into Conserved Framework Regions. (1994) Biochemistry, 33, 5451-5459.
[13] Jung, S. H., Pastan, I. and Lee, B. Design of interchain disulfide bonds in the framework region of the Fv fragment of the monoclonal antibody B3. (1994) Proteins, Struc. Func. Genet., 19, 35-47.
[14] Glockshuber, R., Malia, M., Pfitzinger, I. and Plu\ddot{ }ckthun, A. A comparison of strategies to stabilize immunoglobulin Fv-fragments. (1990) Biochemistry, 29, 1362-1367.
[15] Zhu, Z., Presta, L. G., Zapata, G. and Carter, P. Remodeling domain interfaces to enhance heterodimer formation. (1997) Prot. Sci., 6, 781-788.
[16] Philip H. Tan, Brenda M. Sandmaier, Patrick S. Stayton. Contributions of a Highly Conserved VH VL Hydrogen Bonding Interaction to scFv Folding Stability and Refolding Efficiency. Biophys J. 1998 September; 75(3): 1473-1482.
[17] Zhu Z, Carter P. Identification of heavy chain residues in a humanized anti-CD3 antibody important for efficient antigen binding and T cell activation. J Immunol. 1995 Aug. 15; 155(4):1903-10.
[18] U.S. Pat. No. 7,112,324B1
[19] Ulrich Brinkmann, Yoram Reiter, Sun-hee Jung, etc. A recombinant immunotoxin containing a disulfide-stabilized Fv fragment. PNAS. 1993 Vol. 90, pp. 7538-7542, August
[20] Glockshuber, R., Malia, M., Pfitzinger, I. and Pluckthun, A. A comparison of strategies to stabilize immunoglobulin Fv-fragments. (1990) Biochemistry, 29, 1362-1367.
[21] Igawa T, Tsunoda H, Kikuchi Y, etc. VH/VL interface engineering to promote selective expression and inhibit conformational isomerization of thrombopoietin receptor agonist single-chain diabody. Protein Eng Des Sel. 2010 August; 23(8):667-77.
[22] WO2006106905 (A1)
[23] Gunasekaran K, Pentony M, Shen M, etc. Enhancing antibody Fc heterodimer formation through electrostatic steering effects: applications to bispecific molecules and monovalent IgG. J Biol Chem. 2010 Jun. 18; 285(25):19637-46.
[24] Liu Z, Leng E C2, Gunasekaran K3, etc. A novel antibody engineering strategy for making monovalent bispecific heterodimeric IgG antibodies by electrostatic steering mechanism. J Biol Chem. 2015 Mar. 20; 290(12):7535-62.
[25] US20140154254A1

### EXAMPLES

The present invention is further exemplified, but not limited, by the following and Examples that illustrate the preparation of the compounds of the invention.

### Example 1

### Antibody Construction

In the construction of DICAD molecule CD19×CD3, Fv sequences for CD3 and CD19 are as below.

CD3: UCHT1. Zhu Z, Carter P. Identification of heavy chain residues in a humanized anti-CD3 antibody important for efficient antigen binding and T cell activation. J Immunol. 1995 Aug. 15; 155(4):1903-10, herein is incorporated by reference.

VH:
(SEQ ID NO.: 6)
EVQLVESGGGLVQPGGSLRLSCAASGYSFTGYTMNWVRQAPGKGLEWVAL
INPYKGVSTYNQKFKDRFTISVDKSKNTAYLQMNSLRAEDTAVYYCARSG
YYGDSDWYFDVWGQGTLVTVSS
VL:
(SEQ ID NO.: 7)
DIQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFGQ
GTKVEIK

CD19: HD37. U.S. Pat. No. 7,112,324,B1, herein is incorporated by reference.

VH:
(SEQ ID NO.: 8)
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTSVTVSS
VL:
(SEQ ID NO.: 9)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIK

Molecules were constructed as the following:
Peptide 1: CD19VL-linker-CD3VH-hinge-CH2-CH3;
Peptide 2: CD3VL-linker-CD19VH.

Point mutations were introduced at designated sites in FR of VH and VL domains, as listed in the Table 3 below. To be specific, for each sample (001-062) the upper line is for Peptide 1 and the lower line is for Peptide 2.

TABLE 3

| | Mutation List | | | | Mutation List | | |
|---|---|---|---|---|---|---|---|
| Sample | VL | VH | linker | Sample | VL | VH | linker |
| 001 | wt | wt | RTVAA | 016 | Q38D, | Q39D, G44C, | RTVAA |
| | | | SEQ ID | | Q100C, Y87W | L45A | SEQ ID |
| | | | NO: 1 | | | | NO: 1 |
| | wt | wt | RTVAA | | Q38K, | Q39K, G44C, | RTVAA |
| | | | SEQ ID | | Q100C, Y87W | L45A | SEQ ID |
| | | | NO: 1 | | | | NO: 1 |
| 002 | wt | L45A | RTVAA | 017 | Q38D, | Q39D, G44C, | RTVAA |
| | | | SEQ ID | | Q100C | L45A, Q105A | SEQ ID |
| | | | NO: 1 | | Y87W, A43Y | | NO: 1 |
| | Y87W | wt | RTVAA | | Q38K, | Q39K, G44C, | RTVAA |
| | | | SEQ ID | | Q100C, | L45A, Q105A | SEQ ID |
| | | | NO: 1 | | Y87W, A43Y | | NO: 1 |
| 003 | Q100C | L45A | RTVAA | 018 | Q38D, | Q39D, G44C, | RTVAA |
| | | | SEQ ID | | Q100C, | L45A, Q105K | SEQ ID |
| | | | NO: 1 | | Y87W, A43K | | NO: 1 |
| | Y87W | G44C | RTVAA | | Q38K, | Q39K, G44C, | RTVAA |
| | | | SEQ ID | | Q100C, | L45A, Q105D | SEQ ID |
| | | | NO: 1 | | Y87W, A43D | | NO: 1 |
| 004 | wt | G44C | RTVAA | 23 | Q38D, Q100C | Q39D | RTVAA |
| | | | SEQ ID | | | | SEQ ID |
| | | | NO: 1 | | | | NO: 1 |
| | Q100C | wt | RTVAA | | Q38K | Q39K, G44C | RTVAA |
| | | | SEQ ID | | | | SEQ ID |
| | | | NO: 1 | | | | NO: 1 |
| 005 | Q100C | G44C | RTVAA | 24 | Q38D | Q39D, G44C, | RTVAA |
| | | | SEQ ID | | | Q105D | SEQ ID |
| | | | NO: 1 | | | | NO: 1 |
| | Q100C | G44C | RTVAA | | Q38K, Q100C, | Q39K | RTVAA |
| | | | SEQ ID | | A43K | | SEQ ID |
| | | | NO: 1 | | | | NO: 1 |
| 006 | Q100C | G44C, | RTVAA | 25 | Q38D | Q39D, G44C | (G2S)3 |
| | | L45A | SEQ ID | | | | |
| | | | NO: 1 | | | | |
| | Q100C, Y87W | G44C | RTVAA | | Q38K, Q100C | Q39K | (G2S)3 |
| | | | SEQ ID | | | | |
| | | | NO: 1 | | | | |
| 007 | wt | Q39D | RTVAA | 26 | Q38D, Q100C | Q39D, L45A | (G2S)3 |
| | | | SEQ ID | | | | |
| | | | NO: 1 | | | | |
| | Q38K | wt | RTVAA | | Q38K, Y87W | Q39K, G44C | (G2S)3 |
| | | | SEQ ID | | | | |
| | | | NO: 1 | | | | |
| 008 | Q38D | Q39D | RTVAA | 37 | Q38D | Q39D, G44C, 62E | RTVAA |
| | | | SEQ ID | | | | SEQ ID |
| | | | NO: 1 | | | | NO: 1 |
| | Q38K | Q39K | RTVAA | | Q38K, | Q39K | RTVAA |
| | | | SEQ ID | | Q100C, D1R | | SEQ ID |
| | | | NO: 1 | | | | NO: 1 |
| 009 | Q38D | Q39D, | RTVAA | 38 | wt | Q39D, Q105C | RTVAA |
| | | G44C | SEQ ID | | | | SEQ ID |
| | | | NO: 1 | | | | NO: 1 |
| | Q38K, Q100C | Q39K | RTVAA | | Q38K, A43C | wt | RTVAA |
| | | | SEQ ID | | | | SEQ ID |
| | | | NO: 1 | | | | NO: 1 |
| 010 | Q38D, Q100C | Q39D, | RTVAA | 39 | wt | Q39D, L45A, Q105C | RTVAA |
| | | G44C | SEQ ID | | | | SEQ ID |
| | | | NO: 1 | | | | NO: 1 |
| | Q38K, Q100C | Q39K, | RTVAA | | Q38K, Y87W, | wt | RTVAA |
| | | G44C | SEQ ID | | A43C | | SEQ ID |
| | | | NO: 1 | | | | NO: 1 |
| 011 | Q38D | Q39D, | RTVAA | 40 | P44D | W103D, G44C | RTVAA |
| | | L45A | SEQ ID | | | | SEQ ID |
| | | | NO: 1 | | | | NO: 1 |
| | Q38K, Y87W | Q39K | RTVAA | | P44K, Q100C | W103K | RTVAA |
| | | | SEQ ID | | | | SEQ ID |
| | | | NO: 1 | | | | NO: 1 |
| 012 | Q38D, Q100C | Q39D, | RTVAA | 43 | wt | Q39D, G44C | (G2S)3 |
| | | L45A | SEQ ID | | | | |
| | | | NO: 1 | | | | |
| | Q38K, Y87W | Q39K, | RTVAA | | Q38K, Q100C | wt | (G2S)3 |
| | | G44C | SEQ ID | | | | |
| | | | NO: 1 | | | | |
| 013 | Q38D, Q100C | Q39D, | RTVAA | 44 | wt | W103D, G44C | (G2S)3 |
| | | G44C, | SEQ ID | | | | |
| | | L45A | NO: 1 | | | | |
| | Q38K, Q100C, | Q39K, | RTVAA | | P44K, Q100C | wt | (G2S)3 |
| | Y87W | G44C | SEQ ID | | | | |
| | | | NO: 1 | | | | |
| 014 | Q38D, A43C | Q39D, | RTVAA | 45 | wt | Q39D, 62E, Q105D, | (G2S)3 |
| | | Q105C, | SEQ ID | | | G44C | |
| | | L45A | NO: 1 | | | | |
| | Q38K, A43C, | Q39K, | RTVAA | | Q38K, D1R, | wt | (G2S)3 |
| | Y87W | Q105C | SEQ ID | | A43K, Q100C | | |
| | | | NO: 1 | | | | |
| 015 | Q38D, Q100C, | Q39D, | RTVAA | 46 | A43D, D1K | Q105D, G44C, 62K | (G2S)3 |
| | A43Y | G44C, | SEQ ID | | | | |
| | | Q105A | NO: 1 | | | | |
| | Q38K, Q100C, | Q39K, | RTVAA | | A43K, | Q105K, 62D | (G2S)3 |
| | A43Y | G44C, | SEQ ID | | Q100C, D1D | | |
| | | Q105A | NO: 1 | | | | |
| 61 | A43D | Q39C, | (G2S)3 | 60 | Q100D | Q39C, G44D | (G2S)3 |
| | | Q105D | | | | | |
| | Q38C, A43K | Q105K | (G2S)3 | | Q38C, Q100K | G44K | (G2S)3 |
| 62 | A43D, Q100K | Q39C, | (G2S)3 | | | | |
| | | Q105D, | | | | | |
| | | G44K | | | | | |
| | Q38C, A43K, | Q105K, | (G2S)3 | | | | |
| | Q100D | G44D | | | | | |

Sequences were optimized for codons using OptimumGene before being synthesized. Target genes were initially constructed in pUC57 vectors and then sub-cloned into pTGE5 vectors. DNAs for transfection were prepared by Maxipreps. CHO3E7 cells were cultured and passed at 0.3×10⁶ cell/ml. Transfection was conducted when cell density reached 1.8-2.5×10⁶ cells/ml. 300 μl of DNAs for heavy and light chains, respectively, were first added to 50 ml of Freestyle CHO culture medium and gently mixed. 3 mg of PEI transfection reagent was added later and gently mixed for 3 more minutes. The mixture was allowed to settle at 37 °C for 7 minutes before being added to 450 ml of cell suspension, making a total volume of 500 ml. 24 hours later, 25 ml of TN1 stock (200 g/L) was added to the mixture. 1 ml of suspension was taken for examination at days 1, 3 and 5 respectively post transfection. 50 μl of each sample was used for cell counting, and the rest was centrifuged for 5 minutes at 3000 rpm before the supernatant was kept at −20 °C. On day 6, the culture was harvested and centrifuged for 30 minutes at 5500 rpm. Supernatant was separated, filtered through a 0.22 m filter and further purified for proteins. Columns: 5 ml Monofinity A Resin (GenScript, Cat. No. L00433) column; Balancing Buffer A: 20 mM PB, 150 mM NaCl, pH7.2; Washing Buffer B: 50 mM citric acid, pH3.5; Neutralizing Buffer C: 1M Tris-HCl, pH9.0; Flow: 2 ml/min; Gradient: 100% gradient wash. Post fractionation, 0.155 ml of Neutralizing Buffer C was added to each 1 ml fraction. Collected protein solution was dialyzed in PBS, pH7.2 for 16 hours at 4 °C.

Antibody #1 was constructed combining sequences HD37 and UCHT1 using methods described above. Using Antibody #1 as a model system, different ways of modifications at the VH-VL interface were engaged during construction and their influences on characters and activity of antibodies were tested. Modifications experimented with included: cysteine mutations on FR of VH and VL to form disulfide bonds (VL43-VH105, VL100-VH44); A-W mutations on FR of VH and VL, respectively, to form the KIH structure (knob into hole, VL87-VH45); mutations for amino acids with paired charges on FR of VH and VL to establish electrostatic interaction in between (VL38-VH39).

Molecular engineering designs were as such: to introduce disulfide bonds (#4), charged residues in pair (#7), and KIH (#2) at the VH-VL interface; to combine mutations for KIH and disulfide bonds (#3), KIH and paired charged residues (#11, #12, #26, etc.), and paired charged residues and disulfide bonds (#43, #38, #9, #25, etc.), respectively, at the VH-VL interface; and to simultaneously introduce modifications for KIH, disulfide bonds, and paired charged residues (#39, etc.) at the VH-VL interface. Above examples were screened for designs that resulted in product with the best stability and purity.

To further determine site specificity of mutations for disulfide bonds and paired charged residues, more molecules were constructed and examined, including 43(L)-105(H) and 100(L)-44(H) mutated for KIH or paired charged residues, and 38(L)-39(H) mutated for disulfide bond or KIH.

SDS-Page: Samples were analyzed by SDS-PAGE followed by Commassie blue staining.

Parental antibodies of the CD19×CD3 bispecific antibody: HD37 (#21, anti-CD19) and UCHT1 (#20, anti-CD3) were expressed at the same time and used as controls.

Non-reduced SDS-PAGE revealed a 100 KD band and a 25 KD band for #1 which was without any mutations. Results remained the same when KIH (#2) or paired charged residues (#7) were introduced. When disulfide bonds were introduced (#3), however, a 155 KD band appeared, indicative of covalent interaction between Peptide 1 and Peptide 2; co-presence of a 25 KD band suggested that non-covalent interaction still existed. On the other hand, combination of mutations for paired charged residues and KIH (#11, #12, #26, etc.) failed to change properties of antibody #1. Combination of disulfide bonds and KIH mutations resulted in similar effect as with KIH or paired charged residues alone. When combination of mutations for paired charged residues and disulfide bonds (#9, #25, #43) were applied, only a 155 KD band appeared in non-reduced SDS-PAGE with high purity and no sign of non-covalent interaction. Both antibodies #9 and #25 showed similar purity and molecular weight as the parental antibodies #20 and #21, under either reduced or non-reduced conditions. Further modifications such as introduction of more paired charged residues or KIH did not improve purity; on the contrary, the modifications caused a decrease in expression level of the antibodies. Either 5-amino acid fragment RTVAA (SEQ ID NO: 1) or 9-amino acid fragment (G₂S)₃ as the linker did not significantly influence purity of the product, however, the linker region did affect expression level of the antibodies.

With disulfide bonds introduced to the interface of one VL-VH pair, further introduction of disulfide bonds to the interface of the second pair of VL-VH resulted in large quantities of polymers in the product, regardless of other modifications made to the second VL-VH pair. With disulfide bonds and paired charged residues introduced to the interface of one VL-VH pair, further introduction of paired charged residues to the interface of the second pair of VL-VH improved purity of the product while caused certain decrease in expression level of the product.

In all, the preferred method of molecule construction by DICAD is to covalently connect one pair of VL and VH with disulfide bonds facilitated by paired charged residues at the interface of the FR, and to link VL1 and VH2 regions and VL2 and VH1 regions both using peptides of 5-9 amino acids.

### Example 2

Stability: 12 samples were tested for stability. Stability at 2-8 °C: samples were let sit at 5 °C 3 °C for 10 days and examined at Day 0 and Day 10, respectively. Stability at 25 °C: samples were let sit at 25 °C±2 °C for 10 days and examined at Day 0 and Day 10, respectively. See Table 4.

TABLE 4

| Subject | Day 0 | 2-8 °C/10 days | 25 °C/10 days |
|---|---|---|---|
| SEC-HPLC | √ | √ | √ |
| IEC | √ | √ | √ |
| SDS-N | √ | √ | √ |
| SDS-R | √ | X | X |
| DSC | X | X | X |
| cIEF | √ | X | X |

### A. Stability at 2-8 °C

SDS-PAGE results: Commassie-stained SDS-PAGE gels were scanned using a GS-200 scanner and images were analyzed in Image Lab 5.2.1 for protein purity. The results are summarized in Table 5.

TABLE 5
Protein purity calculated from non-reduced SDS-PAGE results

| Sample | D 0 | D 10 (2-8 °C) |
|---|---|---|
| 3# | 87.9% | 81.9% |
| 7# | 73.3% | 62.2% |
| 9# | 99.4% | 96.9% |
| 11# | 61.4% | 67.9% |
| 20# | 97.3% | 98.0% |
| 21# | 100.0% | 100.0% |
| 25# | 100.0% | 99.7% |
| 38# | 95.9% | 95.7% |
| 39# | 96.7% | 96.2% |
| 40# | 100.0% | 99.8% |
| 43# | 99.4% | 99.2% |
| 44# | 100.0% | 99.5% |

### SEC-HPLC (Size Exclusion-High Performance Liquid Chromatography) Results

Protein purity calculated from SEC-HPLC results are shown in Table 6.

TABLE 6

| | D 0 | | | D 10 (2-8 °C) | | |
|---|---|---|---|---|---|---|
| | | AUC | % | | AUC | % |
| | Retention | (main | (main | Retention | (main | (main |
| Sample | (min) | peak) | peak) | (min) | peak) | peak) |
| 3# | 6.93 | 3804.8 | 49.21i | 7.032 | 3723.8 | 49.337 |
| 7# | 6.86 | 5276.7 | 67.35 | 6.98 | 5507.2 | 71.343 |
| 9# | 6.87 | 7671.8 | 93.91 | 7.002 | 7644.5 | 92.485 |
| 11# | 6.94 | 8026.1 | 90.5 | 7.063 | 7966.2 | 90.061 |
| 20# | 6.87 | 4922.9 | 79.85 | 6.994 | 5036 | 81.352 |
| 21# | 6.7 | 4996.6 | 80.44 | 6.811 | 5749.7 | 90.183 |
| 25# | 6.86 | 6815.7 | 96.31 | 6.985 | 6626.3 | 96.322 |
| 38# | 6.83 | 5833 | 87.52 | 6.964 | 5800.2 | 88.025 |
| 39# | 6.88 | 7516.5 | 89.56 | 7.026 | 7555.6 | 90.292 |
| 40# | 6.79 | 6084.6 | 88.96 | 6.94 | 6160.3 | 90.135 |
| 43# | 6.79 | 6254.7 | 91.9 | 6.946 | 6171.8 | 91.908 |
| 44# | 6.78 | 7225.4 | 92.62 | 6.929 | 7104.8 | 93.159 |

IEC results are shown in Table 7.

TABLE 7

| | |
|---|---|
| D 0 | D 10 (2-8 °C) |

| Sample | acidic | main | basic | acidic | main | basic |
|---|---|---|---|---|---|---|
| 3# | 18.95 | 28.65 | 52.38 | 21.14 | 33.82 | 45.06 |
| 7# | 30.88 | 21.75 | 47.36 | 40.23 | 29.99 | 29.79 |
| 9# | 36.62 | 28.38 | 34.98 | 37.85 | 33.06 | 29.1 |
| 11# | 25.97 | 27.05 | 46.98 | 30.37 | 31.11 | 38.51 |
| 20# | 45.66 | 28.68 | 25.65 | 48.24 | 30.31 | 21.46 |
| 21# | 33.62 | 26.85 | 39.51 | 40.47 | 31.59 | 27.95 |
| 25# | 51.72 | 31.64 | 16.63 | 53.26 | 29.24 | 17.5 |
| 38# | 38.06 | 30.71 | 31.22 | 39.04 | 31.24 | 29.73 |
| 39# | 36.16 | 30.78 | 31.47 | 37.47 | 32.05 | 30.49 |
| 40# | 39.65 | 32.68 | 27.67 | 39.13 | 34.64 | 26.23 |
| 43# | 40.55 | 31.06 | 28.37 | 38.2 | 30.34 | 31.46 |
| 44# | 47.27 | 30.89 | 21.81 | 43.68 | 30.75 | 25.57 |

### Stability at 25 °C;

SDS-PAGE results are shown in Table 8.

TABLE 8
Protein purity calculated from non-reduced SDS-PAGE results

| Sample | D 0 | D 10 (25 °C) |
|---|---|---|
| 3# | 87.9% | 83.8% |
| 7# | 73.3% | 67.3% |
| 9# | 99.4% | 99.3% |
| 11# | 61.4% | 46.9% |
| 20# | 97.3% | 94.3% |
| 21# | 100.0% | 99.4% |
| 25# | 100.0% | 99.4% |
| 38# | 95.9% | 94.3% |
| 39# | 96.7% | 94.0% |
| 40# | 100.0% | 97.8% |
| 43# | 99.4% | 98.1% |
| 44# | 100.0% | 98.9% |

SEC-HPLC results are summarized in Table 9.

TABLE 9
Protein purity calculate from SEC-HPLC results

| | D 0 | | | D 10 (25 °C) | | |
|---|---|---|---|---|---|---|
| | | AUC | % | | AUC | % |
| | Retention | (main | (main | Retention | (main | (main |
| Sample | (min) | peak) | peak) | (min) | peak) | peak) |
| 3# | 6.93 | 3804.8 | 49.21 | 7.039 | 3913 | 50.29 |
| 7# | 6.86 | 5276.7 | 67.35 | 6.991 | 7228.7 | 93.412 |
| 9# | 6.87 | 7671.8 | 93.91 | 7.014 | 7077.9 | 92.256 |
| 11# | 6.94 | 8026.1 | 90.5 | 7.086 | 5853.2 | 90.495 |
| 20# | 6.87 | 4922.9 | 79.85 | 6.999 | 5614.6 | 82.506 |
| 21# | 6.7 | 4996.6 | 80.44 | 6.82 | 6266.1 | 98.343 |
| 25# | 6.86 | 6815.7 | 96.31 | 6.985 | 6591.5 | 97.221 |
| 38# | 6.83 | 5833 | 87.52 | 6.961 | 6174.6 | 94.913 |
| 39# | 6.88 | 7516.5 | 89.56 | 7.019 | 7814.3 | 94.878 |
| 40# | 6.79 | 6084.6 | 88.96 | 6.925 | 6485.9 | 96.438 |
| 43# | 6.79 | 6254.7 | 91.9 | 6.938 | 6297.9 | 95.007 |
| 44# | 6.78 | 7225.4 | 92.62 | 6.919 | 7349.8 | 96.666 |

Stability of 12 samples was analyzed by SEC as described below. Samples were centrifuged for 5 minutes at 10000 rpm, 15 °C. Supernatants were removed and analyzed. The SEC parameters are: Phase A: 100 mM PBS pH6.7; Phase B: double-distilled water; Flow velocity: 0.35 ml/min; Wavelength: 280 nm; Temperature: room temperature; Sample wash-off time: 20 minutes; Loading amount: 20 g.

TABLE 10
Protein purity calculated from SEC-HPLC results

| | | | D 10 | | D 10 |
|---|---|---|---|---|---|
| | | | (2-8 °C) | | (25 °C) |
| | | D 10 | Purity | D 10 | Purity |
| Samlpe | D 0 | (2-8 °C) | change | (25 °C) | change |
| 3# | 49.20% | 49.34% | −0.14% | 50.29% | −1.09% |
| 7# | 67.35% | 71.34% | −3.99% | 93.41% | −26.06% |
| | 76.81%* | 77.62%* | — | 96.12%* | — |
| 9# | 93.91% | 92.48% | 1.43% | 92.26% | 1.65% |
| 11# | 90.49% | 90.06% | 0.43% | 90.49% | 0.00% |
| 20# | 79.85% | 81.35% | −1.50% | 82.51% | −2.66% |
| 21# | 80.44% | 90.18% | −9.74% | 98.34% | −17.9% |
| 25# | 96.31% | 96.32% | −0.01% | 97.22% | −0.91% |
| 38# | 87.52% | 88.02% | −0.50% | 94.91% | −7.39% |
| 39# | 89.56% | 90.29% | −0.73% | 94.88% | −5.32% |
| 40# | 88.96% | 90.14% | −1.18% | 96.44% | −7.48% |
| 43# | 91.90% | 91.91% | −0.01% | 95.01% | −3.11% |
| 44# | 92.62% | 93.16% | −0.54% | 96.67% | −4.05% |

Note:
*samples were first examined on Day 10, let sit for 6 days at 4 °C and re-examined.

The SEC-HPLC results (stability assay). 12 samples were let sit for 10 days at 2-8 °C or 25 °C and no significant change in protein purity was observed (significant: >1%). Sample 47 was re-examined later; purity increased/aggregates decreased as sitting time and temperature increased, suggesting a role of temperature in dissociation of aggregates. A 25 KID band (20-30% of total mass) of a light chain was observed in non-reduced SDS-PADE for samples 3, 7 and 11 respectively but failed to be detected by SEC-HPLC, suggesting that the light chain could be associated with full-length molecules and was only dissociated during SDS-PAGE.

### CEX-HPLC (Cation Exchange-High Performance Liquid Chromatography)

Samples were centrifuged for 5 minutes at 10000 rpm, 15 °C. Supernatants were removed and analyzed. The CEX-HPLC parameters are: Phase A: 20 mM MES, 20 mM NaCl pH5.6; Phase B: 20 mM MES, 300 mM NaCl pH5.6; Gradient: 20-60%; Flow velocity: 1.0 ml/min; Wavelength: 280 nm; Temperature: room temperature; Sample wash-off time: 110 minutes; Loading amount: 20 μL.

Samples were let sit for 10 days at 2-8 °C or 25 °C before being processed and examined. Under both conditions samples #3, #7, #9, #20, #21 and #25 showed increased proportion of acidic variants and decrease proportion of basic variants, and the change was not significantly influenced by temperature. Sample #11 showed both an increase in acidic variants and a decrease in basic variants under 2-8 °C, while no significant change was observed in either under 25 °C. Sample #25 showed slight increase in both acidic and basic variants, as well as a decrease in proportion of the main peak. Sample #44 showed decrease acidic variants and increased basic variants under both conditions. No significant change was observed for sample #38, #39, #40 and #43 under either condition.

IEC results are shown in Table 11.

TABLE 11
IEC results

| | |
|---|---|
| D 0 | D 10 (25 °C) |

| Samples | acidic | main | basic | acidic | main | basic |
|---|---|---|---|---|---|---|
| 3# | 18.95 | 28.65 | 52.38 | 22.4 | 32.77 | 44.9 |
| 7# | 30.88 | 21.75 | 47.36 | 41.6 | 30.18 | 28.3 |
| 9# | 36.62 | 28.38 | 34.98 | 40 | 31.94 | 28.1 |
| 11# | 25.97 | 27.05 | 46.98 | 26.7 | 27.25 | 46.1 |
| 20# | 45.66 | 28.68 | 25.65 | 49 | 30.23 | 20.8 |
| 21# | 33.62 | 26.85 | 39.51 | 42.4 | 32.23 | 25.4 |
| 25# | 51.72 | 31.64 | 16.63 | 52.7 | 29.1 | 18.3 |
| 38# | 38.06 | 30.71 | 31.22 | 39.1 | 30.19 | 30.7 |
| 39# | 36.16 | 30.78 | 31.47 | 37.7 | 31.5 | 30.8 |
| 40# | 39.65 | 32.68 | 27.67 | 39.9 | 33.94 | 26.2 |
| 43# | 40.55 | 31.06 | 28.37 | 38.9 | 29.93 | 31.1 |
| 44# | 47.27 | 30.89 | 21.81 | 43.7 | 30.75 | 25.6 |

cIEF: Samples were dialyzed into 100 mM Tris before being examined as described. Briefly, loading reagent: 200 μL 3M Urea-cIEF Gel, 12 μL Ampholyte solution, 20 μL negative electrode buffer, 2.0 μL positive electrode buffer, pI Marker standards (pI 10.0, 9.5, 5.5, 4.1) 2.0 μL each, mix. De-salted samples were added to the above mix and mix again thoroughly before being loaded. Results were analyzed in 32 karat and shown in Table 12.

TABLE 12
pI Markers

| # | pI Markers |
|---|---|
| 1 | 10.0 |
| 2 | 9.5 |
| 3 | 5.5 |
| 4 | 4.1 |

The cIEF results are shown in Table 13.

TABLE 13
cIEF results

| | pI | | Main Peak 1 | Main Peak 2 |
|---|---|---|---|---|
| Sample | value | pI Range | (Corrected Area)/% | (Corrected Area)/% |
| 3# | NA | 7.82-7.95 | 24.26 | 17.32 |
| 7# | NA | 7.50-7.67 | 38.89 | 38.24 |
| 9# | NA | 7.50-7.67 | 41.50 | 31.03 |
| 11# | NA | 7.55-7.68 | 35.87 | 34.50 |
| 20# | NA | 8.56-8.74 | 36.60 | 36.02 |
| 21# | NA | 7.09-7.19 | 41.64 | 34.20 |
| 25# | 7.35 | NA | 64.70 | 19.91 |
| 38# | NA | 7.54-7.69 | 44.08 | 39.07 |
| 39# | NA | 7.54-7.68 | 38.53 | 38.24 |
| 40# | 7.46 | NA | 50.43 | 17.27 |
| 43# | NA | 7.51-7.68 | 45.69 | 35.49 |
| 44# | 7.25 | NA | 52.39 | 28.09 |

### Example 3

### Differential Scanning Calorimetry (DSC) Results

Differential Scanning Calorimetry (DSC) results are shown in Table 14.

TABLE 14

| | T½ | ΔH | Tonset | Tm1 | Tm2 | Tm3 | Tm4 |
|---|---|---|---|---|---|---|---|
| # | (°C) | (cal/mol) | (°C) | (°C) | (°C) | (°C) | (°C) |
| Sample01 | 15.2 | 1.12E6 | 45.37 | 59.37 | 68.18 | 76.59 | 84.99 |
| Sample03 | 11.2 | 8.71E5 | 50.12 | 66.54 | 84.92 | | |
| Sample07 | 6.41 | 9.64E5 | 47.61 | 59.62 | 68.03 | 74.41 | 84.81 |
| Sample25 | 25.6 | 9.26E5 | 45.08 | 58.28 | 67.48 | 79.08 | |
| Sample43 | 6.8 | 6.88E5 | 49.87 | 59.87 | 67.87 | 77.87 | 85.07 |
| Sample48 | 21.2 | 6.1E5 | 47.27 | 58.88 | 73.27 | 87.27 | |

### Example 4

### Affinity

### Affinity Kinetics Studies Based on Human CD19-Binding Assays

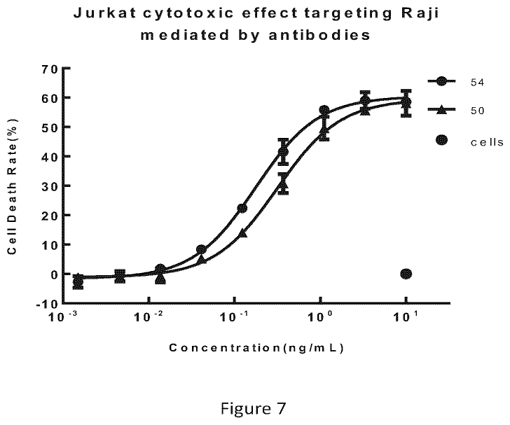

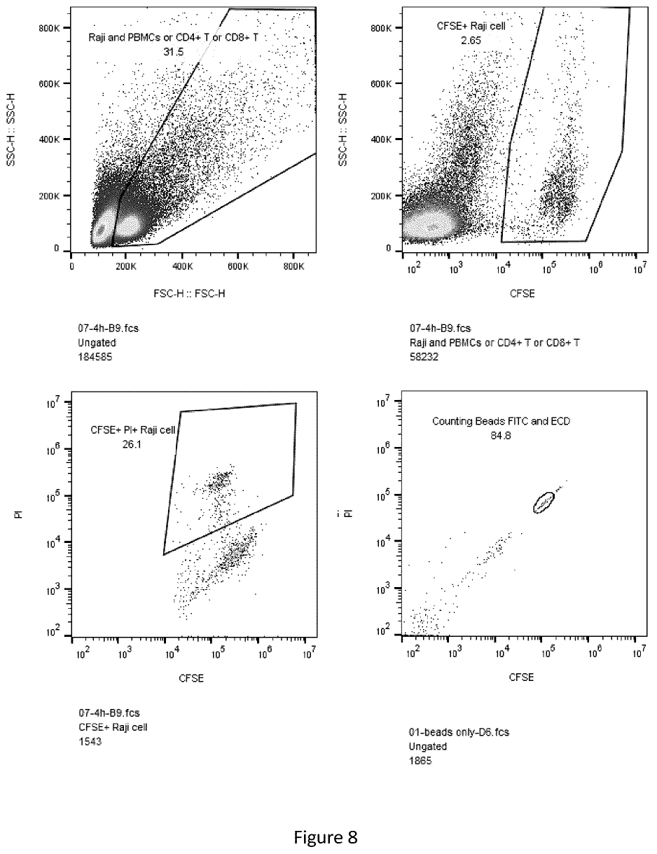

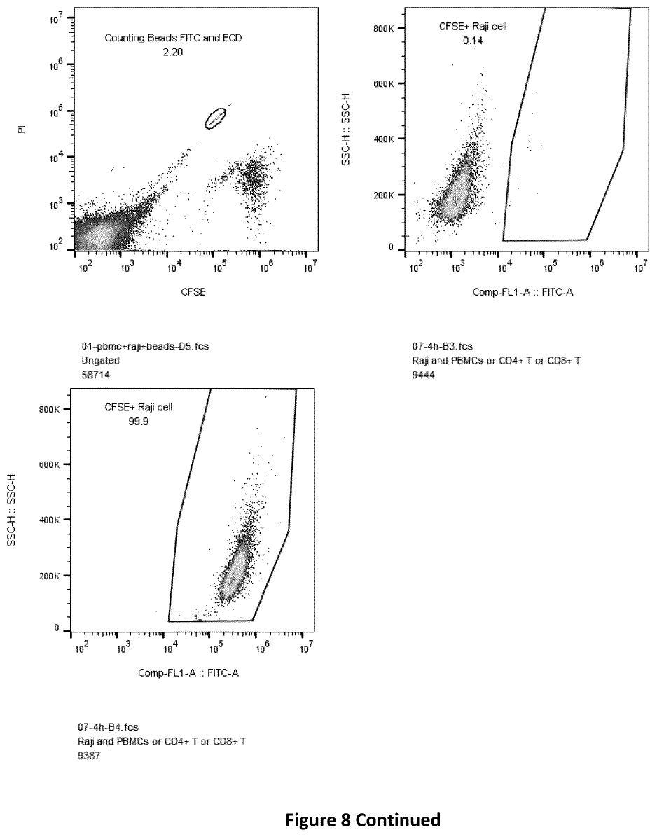

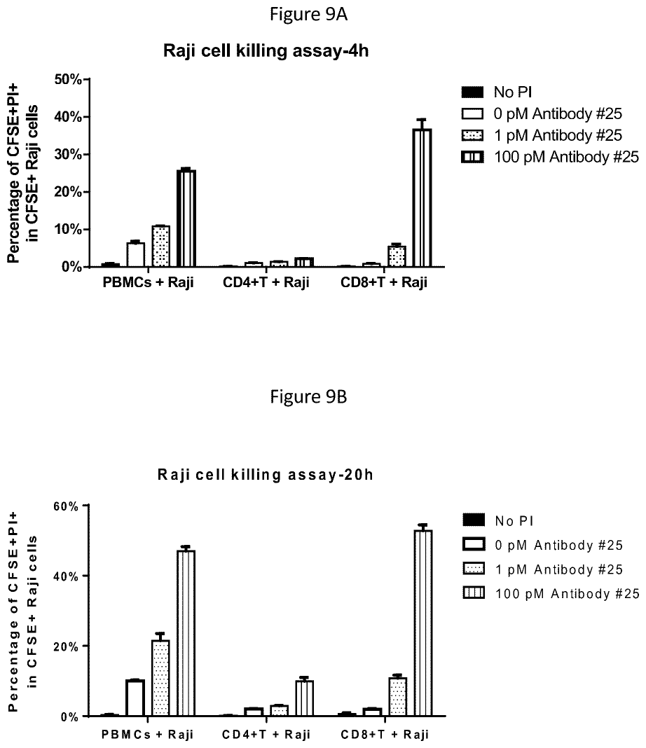

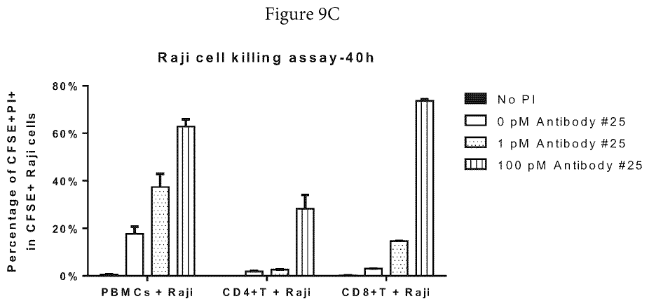

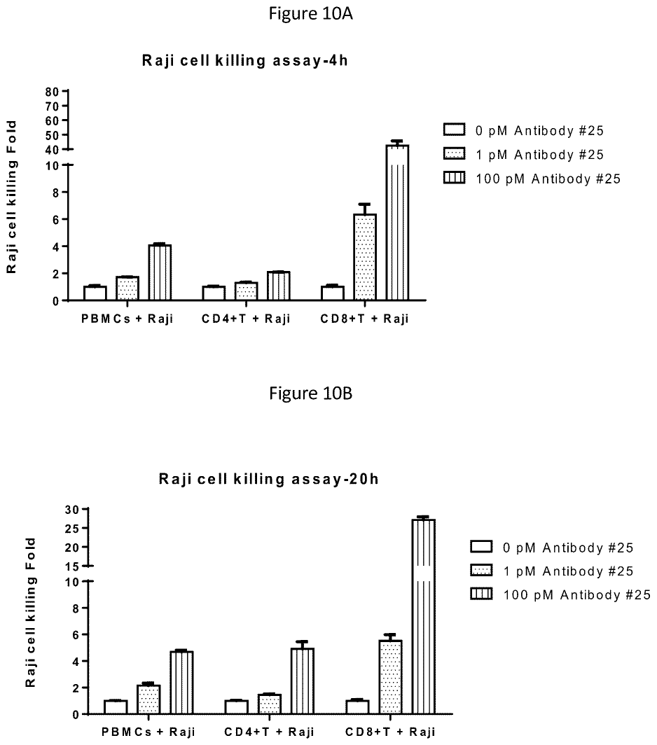

A series of sample antibodies were assayed for SPR (Surface Plasmon Resonance) signals upon human CD19 binding on the Biacore platform. Kₐ, K_{d} and K_{D} were calculated from the experimental results and used to evaluate affinity of the antibodies for human CD19. CD19 molecules as ligands were immobilized to chips conjugated with anti-histine antibodies. Sample antibodies, at 5 different concentrations, were injected to the system to be analyzed. Anti-CD3 antibody UCHT1 and anti-CD19 antibody HD37 were also examined as #20 and #21, respectively. The results are shown in FIG. 7 and Table 15.

TABLE 15

| Antibodies | Ka | Kd | K_{D} |
|---|---|---|---|
| 001 | 2.144E+5 | 1.649E−4 | 7.692E−10 |
| 009 | 7.390E+5 | 1.920E−4 | 2.599E−10 |
| 020 | No binding | | |
| 021 | 3.653E+5 | 1.963E−4 | 5.373E−10 |
| 025 | 8.176E+5 | 8.210E−4 | 1.004E−9 |
| 026 | 2.620E+5 | 5.096E−4 | 1.945E−9 |
| 043 | 3.823E+5 | 2.055E−4 | 5.374E−10 |

### Affinity Kinetics Studies Based on Human CD3-Binding Assays

A series of sample antibodies were assayed for SPR (Surface Plasmon Resonance) signals upon human CD3 binding on the Biacore platform. Ka, Kd and KD were calculated from the experimental results and used to evaluate affinity of the antibodies for human CD3. CD3 molecules as ligands were immobilized to chips conjugated with anti-histine antibodies. Sample antibodies, at 5 different concentrations, were injected to the system to be analyzed.

TABLE 16

| antibodies | Ka | Kd | K_{D} |
|---|---|---|---|
| 009 | 3935 | 6.759E−4 | 1.718E−7 |
| 020 | 911.7 | 3.537E−4 | 3.880E−7 |
| 021 | No-binding | | |
| 025 | 5790 | 2.019E−4 | 3.486E−8 |
| 026 | 5905 | 2.314E−4 | 3.918E−8 |
| 043 | 887.2 | 2.391E−4 | 2.694E−7 |

### Example 5

### Cell Killing Assays

Antibody-mediated killing effect on target cells (Raji cells) was assayed using Jurkat as effector cells. Protocol as described below.

Preparation of effector cells: Jurkat cells were passed at 2×10⁵ cells/mL and used for experiments 4 days after passage. Proper amount of cell suspension was transferred to 50 ml tubes and centrifuged for 5 minutes at 200 g, room temperature. Cells were re-suspended with cell culture medium and examined for cell density and cell survival rate. Cell culture medium was used to adjust cell density to 2×10⁶ live cells/mL before 100 μL per well of cell suspension was added to flat-bottom 96-well plates. Effector cells to target cells ratio (E/T) was 10:1 for the experiments.

Preparation of target cells: Raji cells were passed at 2×10⁵ cells/mL and used for experiments 4 days after passage. Proper amount of cell suspension was transferred to 50 ml tubes and centrifuged for 5 minutes at 200 g, room temperature. Cells were re-suspended with cell culture medium and examined for cell density and cell survival rate. Cell culture medium was used to adjust cell density to 2×10⁵ live cells/mL before 100 μL per well of cell suspension was added to flat-bottom 96-well plates with Raji cells already in.

Preparation of antibodies: stocks for sample antibodies #4, #9, #25 and #49 were diluted in cell culture medium to a starting concentration of 10 ng/mL. The samples were further diluted in series at 1:3 for 10 times (10 concentrations) and 10 μL per well of each working dilution was added to flat-bottom 96-well plates (with Jurkat cells and Raji cells added in advance). Sample #49 was specifically designed for better comparison with Blinatumomab. It was constructed on basis of the BITE structure, linking Fv regions of HD37 and UCHT1 (VLCD19-VHCD19-VHCD3-VLCD3) by linkers that were identical as in Blinatumomab. As a result, sample #49 had a molecular weight of 54 kDa, while the rest all had the same molecular weight of 156 kDa.

The flat-bottom 96-well plates with antibodies, target cells and effector cell were left in the incubator at 37 °C, 5% CO2 for 24 hours before supernatant from each well was collected and assayed for LDH by ELISA.

EC50 of samples #4, #9, #25 and #49 fell in between 0.06-0.16 ng/ml, which after calculation turned to be 6.54×10⁻¹⁰ M, 9.95×10⁻¹⁰ M, 6.00×10⁻¹⁰ M, 1.25×10⁻⁹M, respectively. The samples displayed similar killing effect, suggesting that antibodies with DICAD structure were similar or superior to the ones with BITE structure in terms of their killing effect.

### In Vivo Effect

Antibodies were tested for their in vivo anti-tumor effect in the Jeko-1/NCG Mixeno model. On the starting day (Day 0), 5×10⁶ Jeko-1 cells suspended in 100 μL 1:1 PBS/gel was inoculated in the right flank of animals. 3 days after inoculation (Day 3) 1×10⁷/0.1 mL PBMC were injected into the abdomen of animals. Sample antibodies were administrated when average tumor size reached 100 mm³. 3 antibodies were tested (#1@0.5 mg/kg, #025@0.5 mg/kg, #49@0.5 mg/kg) together with one pH6.0 PBS control group; 6 animals per group. All samples were administrated by intravenous injection into the caudal vein. #1, #25 and vehicle were administrated twice a week for 3 weeks consecutively, while #49 was administrated daily for 10 days. Effect was evaluated based on relative tumor inhibition (TGIRTV), and safety was evaluated on animal weight change and animal death. FIG. 7.

### Example 6

### Raji Cell Killing Mediated by DICAD Antibody #25

Antibody-mediated killing effect on target cells (Raji cells) was assayed using lymphocytes as effector cells. Protocol as described below.

Preparation of effector cells: PBMC were freshly isolated from blood by density gradient centrifugation. CD4+ and CD8+ T cells were further isolated from PBMC respectively using Stemcell isolation kits. PBMC, CD4+ T cells and CD8+ T cells were re-suspended in cell culture medium respectively and examined for cell density and cell survival rate. Cell culture medium was used to adjust cell density to 6×10⁶ live cells/mL before 50 μL per well of cell suspension was added to flat-bottom 96-well plates. Effector cells to target cells ratio (E/T) was 20:1 for the experiments. Cell culture medium: RPMI 1640 (Ginco™, Cat. No. 11875093) suspended with 10% HI-FBS and 1% penicillin-streptomycin.

Preparation of target cells: Raji cells were passed at 2×10⁵ cells/mL and used for experiments 4 days after passage. Proper amount of cell suspension was transferred to 50 ml tubes and centrifuged for 5 minutes at 200 g, room temperature. For flow analysis, cells were stained with 1 pM CSFE in PBS in the dark for 20 minutes and washed twice in PBS+5% HI-PBS. Cells were re-suspended with cell culture medium and examined for cell density and cell survival rate. Cell culture medium was used to adjust cell density to 3×10⁵ live cells/mL before 50 μL per well of cell suspension was added to flat-bottom 96-well plates.

Preparation of antibodies: stock for sample antibody #25 was diluted in cell culture medium to various concentrations. 50 μL of cell culture medium or diluted solution was added to indicated wells to make the final concentrations of 0 pM, 1 pM or 100 pM.

The flat-bottom 96-well plates with antibodies, target cells and effector cells (total volume 150 μL/well) were left in the incubator at 37 °C, 5% CO₂. Samples were taken at 4 h, 20 h and 40 h for examination. Briefly, for the LDH assay samples were centrifuged at 350 g for 5 minutes and supernatant from each well was collected and assayed for LDH by ELISA. For flow analysis after above centrifugation cells were re-suspended and stained with PI. 10 μL of counting beads were added for each well before the samples were analyzed by flow cytometry.

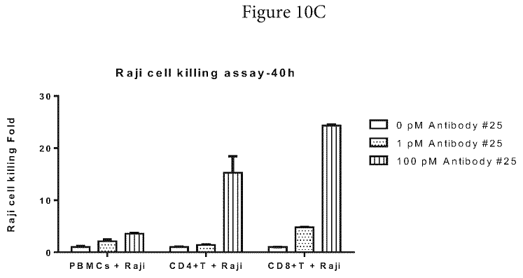

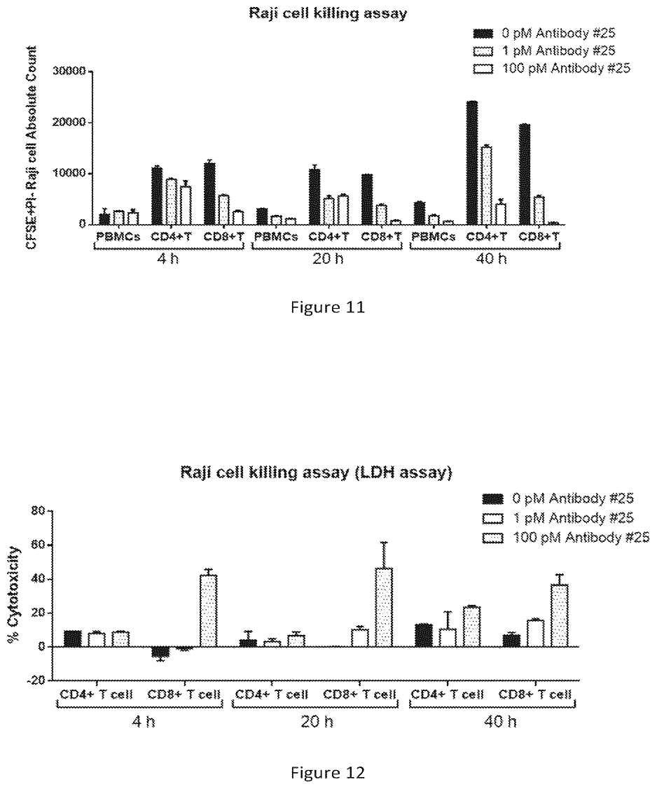

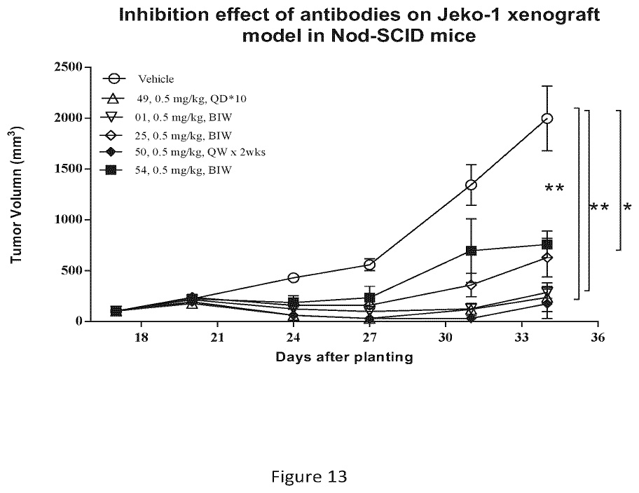

Antibody #25 showed substantial Raji killing effect for all three cell types, e.g. PBMC, CD4+ and CD8+, in a time-dependent and dose-dependent manner; CD8+ T cells resulted the most significant cell death (Figures A&B). In the LDH assay the killing effect seemed to be decreasing towards 40 h. This was likely due to the fact that as the culture last, cell death not-related to antibodies (the noise) was on the rise and affected the accuracy of the assay. FIG. 11 and FIG. 12.

### Example 7

### Antibody Construction

In construction of TRIAD molecule CD19×CD3, Fv sequences for CD3 and CD19 are as below:

CD3: UCHT1
VH(VH3):
(SEQ ID NO.: 6)
EVQLVESGGGLVQPGGSLRLSCAASGYSFTGYTMNWVRQAPGKGLEWVAL
INPYKGVSTYNQKFKDRFTISVDKSKNTAYLQMNSLRAEDTAVYYCARSG
YYGDSDWYFDVWGQGTLVTVSS
VL(VL3):
(SEQ ID NO.: 7)
DIQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFGQ
GTKVEIK
CD19: HD37
VH(VH19):
(SEQ ID NO.: 8)
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTSVTVSS
VL(VL19):
(SEQ ID NO.: 9)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIK

Molecules were constructed as described below in Table 17

TABLE 17

| | Polypeptide | Mutation List | | | |
|---|---|---|---|---|---|
| | Chain | Domain Order | Domain 1 | Domain 2 | CH3 |
| 50 | 1 | VL3-linker-VH19 | Q38K, | Q39K | |
| | | | Q100C | | |
| | 2 | VL19-linker-VH3- | Q38D | Q39D, | T366W, |
| | | hinge-CH2—CH3 | | G44C | S354C |
| | 3 | VH19-CH1-hinge- | wt | wt | Y407V, |
| | | CH2—CH3 | | | T366S, |
| | | | | | L368A, |
| | | | | | Y349C |
| | 4 | VL19-CL | wt | wt | |
| 54 | 1 | VL19-linker-VH3 | Q38K, | Q39K | |
| | | | Q100C | | |
| | 2 | VL3-linker-VH19- | Q38D | Q39D, | T366W, |
| | | hinge-CH2—CH3 | | G44C | S354C |
| | 3 | VH19-CH1-hinge- | wt | wt | Y407V, |
| | | CH2—CH3 | | | T366S, |
| | | | | | L368A, |
| | | | | | Y349C |
| | 4 | VL19-CL | wt | wt | |

The Fab end of Antibody #50 recognized the second antigen (same as that was recognized by VL2-VH2). The Fab end of Antibody #54 recognized the first antigen (same as that was recognized by VL1-VH1). Sequences were optimized for codons using OptimumGene before being synthesized. Target genes were initially constructed in pUC57 vectors and then sub-cloned into pTGE5 vectors. DNAs for transfection were prepared by maxipreps. CHO3E7 cells were cultured and passed at 0.3×10⁶ cell/ml. Transfection was conducted when cell density reached 1.8-2.5×10⁶ cells/ml. 300 ul of DNAs for heavy and light chains, respectively, were first added to 50 ml of Freestyle CHO culture medium and gently mixed. 3 mg of PEI transfection reagent was added later and gently mixed for 3 more minutes. The mixture was allowed to settle at 37 °C for 7 minutes before being added to 450 ml of cell suspension, making a total volume of 500 ml. 24 hours later, 25 ml of TN1 stock (200 g/L) was added to the mixture. 1 ml of suspension was taken for examination at days 1, 3 and 5 respectively post transfection. 50 ul of each sample was used for cell counting, and the rest was centrifuged for 5 minutes at 3000 rpm before the supernatant was kept at −20 °C. On day 6, the culture was harvested and centrifuged for 30 minutes at 5500 rpm. Supernatant was separated, filtered through a 0.22 m filter and further purified for proteins. Columns: 5 ml Monofinity A Resin (GenScript, Cat. No. L00433) column; Balancing Buffer A: 20 mM PB, 150 mM NaCl, pH7.2; Washing Buffer B: 50 mM citric acid, pH3.5; Neutralizing Buffer C: 1M Tris-HCl, pH9.0; Flow: 2 ml/min; Gradient: 100% gradient wash. Post fractionation, 0.155 ml of Neutralizing Buffer C was added to each 1 ml fraction. Collected protein solution was dialyzed in PBS, pH7.2 for 16 hours at 4 °C.

### SDS-Page and Western Blot

The above samples were analyzed by SDS-PAGE followed by Western blot. SDS-PAGE results showed that small amount of aggregates occurred in samples #50 and #54 post Protein A purification. The samples were further purified by SEC and evaluated for their properties and activity.

### Purity Analysis

Samples #50 and #54 were SEC-purified and analyzed for their purity: 99.14% and 99.24% respectively.

### Stability

12 samples were tested for stability: 3,7,9,11,20,21,25,38,39,40,43,44. Stability at 2-8 °C: samples were let sit at 5 °C±3 °C for 10 days and examined at Day 0 and Day 10, respectively. Stability at 25 °C: samples were let sit at 25 °C±2 °C for 10 days and examined at Day 0 and Day 10, respectively.

SDS-PAGE results: Commassie-stained SDS-PAGE gels were scanned using a GS-200 scanner and images were analyzed in Image Lab 5.2.1 to calculate for protein purity. The SDS-PAGE results showed that purity of #54 decreased by 7.4% after sitting 25 °C for 10 days and bands from protein degradation could be observed, while no obvious difference in purity was observed for #50 after sitting for 10 days under either condition. Table 18.

TABLE 18
protein purity calculated from non-reduced SDS-PAGE results

| | | | D 10 | | D 10 |
|---|---|---|---|---|---|
| | | | (2-8 °C) | | (25 °C) |
| | | D 10 | Change of | D 0 | Change of |
| Sample | D 0 | (2-8 °C) | purity | (25 °C) | purity |
| 50# | 96.5% | 95.4% | −1.1% | 94.1% | −2.4% |
| 54# | 97.1% | 96.1% | −1.0% | 89.7% | −7.4% |

SEC-HPLC (Size Exclusion-High Performance Liquid Chromatography) results. SEC-HPLC results showed that after sitting at 2-8 °C for 10 days, purity of #50 slightly decreased (Δ<3%), while purity of #54 increased a little (Δ<2%); after sitting at 25 °C for 10 days, purity of #50 slightly decreased (Δ<4%), while purity of #54 showed a little increase (Δ<2%). Table 19.

TABLE 19
protein purity calculated from SEC-HPLC results

| | | | D 10 | | D 10 |
|---|---|---|---|---|---|
| | | D 10 | (2-8 °C) | D 10 | (25 °C) |
| Sample | D 0 | (2-8 °C) | Δpurity | (25 °C) | Δpurity |
| 50# | 99.847% | 96.854% | 2.993% | 96.536% | 3.311% |
| 54# | 98.412% | 99.708% | −1.296% | 99.726% | −1.314% |

CEX-HPLC (Cation Exchange-High Performance Liquid Chromatography). CEX-HPLC separates molecules based on their net surface charge, using a negatively charged ion exchange resin with an affinity for positive charges. Samples were dialyzed into a buffer before being centrifuged for 5 minutes at 10000 rpm, 15 °C. Supernatants were removed and analyzed.

CEX-HPLC parameters: Phase A: 20 mM MES, 20 mM NaCl pH5.6; Phase B: 20 mM MES, 300 mM NaCl pH5.6; Gradient: 20-60%; Flow velocity: 1.0 ml/min; Wavelength: 280 nm; Temperature: room temperature; Sample wash-off time: 110 minutes; Loading amount: 20 μL.

TABLE 20
IEC results

| | D 0 | | | D 10 (2-8 °C) | | | D 10 (25 °C) | | |
|---|---|---|---|---|---|---|---|---|---|
| | acidic | main | basic | acidic | main | basic | acidic | main | basic |
| 50# | 18.46% | 55.35% | 26.19% | 28.69% | 51.76% | 19.56% | 28.49% | 54.66% | 16.85% |
| 54# | 23.62% | 53.84% | 22.54% | 24.13% | 54.72% | 21.15% | 24.73% | 55.8% | 19.67% |

#50 was allowed to sit at 2-8 °C for 10 days and IEC results showed an increase in acidic frequency (Δ=10.23%), with a decrease in main frequency (Δ=3.59%) and basic frequency (Δ=6.63%). After sitting at 25 °C for 10 days, IEC results of the samples showed an increase in acidic frequency (A=10.03%), with a slight decrease in main frequency (Δ=0.69%) and a decrease in basic frequency (A=9.34%).

#54 was allowed to sit at 2-8 °C for 10 days and IEC results showed a slight increase in acidic frequency (Δ=0.51%), with a slight decrease in main frequency (Δ=0.88%) and basic frequency (A=1.39%). After sitting at 25 °C for 10 days, IEC results of the samples showed a slight increase in acidic frequency (Δ=1.11%), with a slight decrease in main frequency (Δ=1.96%) and some decrease in basic frequency (Δ=2.87%).

cIEF. Samples were dialyzed into 100 mM Tris before being examined as described. Briefly, loading reagent: 200 μL 3M Urea-cIEF Gel, 12 μL Ampholyte solution, 20 μL negative electrode buffer, 2.0 μL positive electrode buffer, pI Marker standards (pI 10.0, 9.5, 5.5, 4.1) 2.0 μL each, mix. De-salted samples were added to the above mix and mix again thoroughly before being loaded. Results were analyzed in 32 karat.

TABLE 21
cIEF results

| Sample | pI value | Main Peak (Corrected Area)/% |
|---|---|---|
| 50# | 7.14 | 34.37 |
| 54# | 7.11 | 45.47 |

Affinity: Affinity kinetics studies based on human CD19-binding assays. A series of sample antibodies were assayed for SPR (Surface Plasmon Resonance) signals upon human CD19 binding on the Biacore platform. Kₐ, K_{d} and K_{D} were calculated from the experimental results and used to evaluate affinity of the antibodies for human CD19. CD19 molecules as ligands were immobilized to chips conjugated with anti-histine antibodies. Sample antibodies, at 5 different concentrations, were injected to the system to be analyzed.

TABLE 22

| | Ka | Kd | K_{D} |
|---|---|---|---|
| HD37 | 1.149E+5 | 4.385E−4 | 3.815E−9 |
| 050 | 2.225E+5 | 3.845E−4 | 1.728E−9 |
| 054 | 2.207E+5 | 1.780E−4 | 8.062E−10 |

### Cell Killing Assays

Antibody-mediated killing effect on target cells (Raji cells) was assayed using Jurkat as effector cells. Protocol as described below:

Preparation of effector cells: Jurkat cells were passed at 2×10⁵ cells/mL and used for experiments 4 days after passage. Proper amount of cell suspension was transferred to 50 ml tubes and centrifuged for 5 minutes at 200 g, room temperature. Cells were re-suspended with cell culture medium and examined for cell density and cell survival rate. Cell culture medium was used to adjust cell density to 2×10⁶ live cells/mL before 100 μL per well of cell suspension was added to flat-bottom 96-well plates. Effector cells to target cells ratio (E/T) was 10:1 for the experiments.

Preparation of target cells: Raji cells were passed at 2×10⁵ cells/mL and used for experiments 4 days after passage. Proper amount of cell suspension was transferred to 50 ml tubes and centrifuged for 5 minutes at 200 g, room temperature. Cells were re-suspended with cell culture medium and examined for cell density and cell survival rate. Cell culture medium was used to adjust cell density to 2×10⁵ live cells/mL before 100 μL per well of cell suspension was added to flat-bottom 96-well plates with Raji cells already in.

Preparation of antibodies: stocks for sample antibodies #50 and #54 were diluted in cell culture medium to a starting concentration of 10 ng/mL. The samples were further diluted in series at 1:3 for 10 times (10 concentrations) and 10p L per well of each working dilution was added to flat-bottom 96-well plates (with Jurkat cells and Raji cells added in advance).

Flat-bottom 96-well plates with antibodies, target cells and effector cell were left in the incubator at 37 °C, 5% CO2 for 24 hours before supernatant from each well was collected and assayed for LDH by ELISA. Results of the assays revealed that EC50 of #50 and #54 were 0.3164 ng/ml and 0.1769 ng/ml, respectively. The two tri-specific antibodies had similar killing effect on target cells.

### In Vivo Effect

Antibodies were tested for their in vivo anti-tumor effect in the Jeko-1/NCG Mixeno model. On the starting day (Day 0), 5×106 Jeko-1 cells suspended in 100 μL 1:1 PBS/gel was inoculated in the right flank of animals. 3 days after inoculation (Day 3) 1×107/0.1 mL PBMC were injected into the abdomen of animals. Sample antibodies were administrated when average tumor size reached 100 mm3. 5 antibodies were tested (#1@0.5 mg/kg, #25@0.5 mg/kg, #50@0.5 mg/kg, #54@0.5 mg/kg, and #49@0.5 mg/kg as control) together with one pH6.0 PBS control group; 6 animals per group. All antibodies tested in the experiment were CD3×CD19 antibodies constructed on different platforms, among which #1 was Di-Diabody, #25 was DICAD, #49 was BITE, #50 and #54 were TRIAD (The Fab end of Antibody #50 recognized the second antigen (same as that was recognized by VL2-VH2). The Fab end of Antibody #54 recognized the first antigen (same as that was recognized by VL1-VH1). All samples were administrated by intravenous injection into the caudal vein. All antibodies and the vehicle were administrated twice a week for 3 weeks consecutively, while #49 (control) was administrated daily for 10 days. Effect was evaluated based on relative tumor inhibition (TGIRTV), and safety was evaluated on animal weight change and animal death.

TGIRTV=1−TRTV/CRTV (%). Relative tumor growth inhibition rate TGIRTV (%):

TRTV/CRTV (%) is relative tumor growth rate, i.e. at a certain time point, the ratio between the tumor volume of the group that received treatment to the tumor volume of the control group that received PBS. TRTV and CRTV are tumor volume (TV) of the treatment group and the control group at a certain time point, respectively.

The experiment ended 34 days after inoculation. All treatment (antibody) groups showed significant inhibition on tumor growth. #50 had a TGIRTV (%) of 92%, which had a clear advantage over all the other molecules being tested including #25 that was constructed on DICAD.

### Example 8

### Antibody Construction

Anti-CD3 antibodies or fragments are commonly used as the binding agent for effector cells in the construction of T cell-engaged cytotoxic antibodies, to induce target-dependent killing activity of T cells. However, anti-CD3 antibodies or fragments can bind to certain functional areas of CD3 (e.g. anti-CD3εγ/εδ by OKT3) or bind in a bivalent mode which result in target-independent T cell activation and subsequent activation of dendritic cells and macrophages in addition to T cell apoptosis, and lead to severe CRS (cytokine release syndrome). Thus anti-CD3 used for the construction of T cell-engaged cytotoxic antibodies should be able to: 1) specifically bind to membrane CD3 and 2) bind to CD3 in a monovalent manner. We constructed DICAD molecule CD19×CD3 (1:1) as the following:
Polypeptide A (SEQ ID NO.: 10): CD19 VL-linker-CD3 VH
Polypeptide B (SEQ ID NO.: 11): CD3 VL-linker-CD19 VH-hinge-CH2-CH3
Polypeptide C (SEQ ID NO.: 12): Hinge-CH2-CH3

DNAs for three polypeptides (sequences for polypeptides A, B and C were as below) were included in vectors and transfected into CHO3E7 cells for transient expression. Expressed protein was purified using Protein A and purity of the product was over 95%.

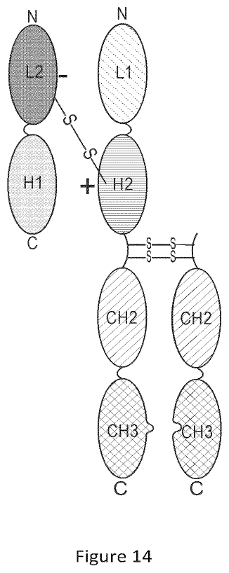

Mutations on CD19 VL-linker-CD3 VH of Polypeptide A and CD3 VL-linker-CD19 VH of Polypeptide B are made according to previous examples to form covalent connection between the two chains (A&B). CH3 of Polypeptide B and CH3 of Polypeptide C are connected using the KIH (knobs-into-holes) structure to increase yield and proportion of heterodimers. FIG. 14 depicts diagrammatically the structure of DICAD as constructed according to the present example. Fv sequences for CD3 and CD19 were listed in sequence listing (CD19 VL: SEQ ID NO.: 13; CD19 VH: SEQ ID NO.: 14; CD3 VL: SEQ ID NO.: 15; CD3 VH: SEQ ID NO.: 16).

Point mutations were introduced at designated sites in FR of VH and VL domains of CD3 and CD19, as listed in the Table 23 below. To be specific, the upper line is for Peptide A and the lower line is for Peptide B.

TABLE 23

| | Mutation List | | |
|---|---|---|---|
| Sample | VL | VH | linker |
| 063 | Q38K, G100C | Q39K | RTVAA |
| | | | (SEQ ID |
| | | | NO: 1) |
| | Q38D, G44C | Q39D | RTVAA |
| | | | (SEQ ID |
| | | | NO: 1) |

### Example 9

### Affinity

Binding affinity for human CD3ε or human CD19 of the CD3×CD19 bispecific antibody was tested by BIAcore, and Kₐ, K_{d} and K_{D} values of the antibody were calculated based on the results. His-tagged human CD19 was used as ligand and captured on chips conjugated with anti-histine antibodies. Candidate molecules in five different concentrations were analyzed for their affinity for CD19. Affinity for human CD3ε was examined using the same method. Results are listed in Table 24 below.

TABLE 24

| Analyst | Kₐ (1/Ms) | K_{d} (1/s) | K_{D} (M) |
|---|---|---|---|
| CD3 | 4.876E+5 | 0.01530 | 3.137E−8 |
| CD19 | 3.653E+5 | 6.686E−4 | 1.830E−9 |

### CD3×CD19 Bispecific Antibody #63 Mediated Killing of Raji Cells by PBMC In Vitro in a Dose-Dependent Manner

Lymphocytes were used as effector cells to assess the antibody-mediated killing of Raji cells (target cells). Protocols are as below:

Preparation of effector cells: PBMCs were freshly isolated from human blood by density gradient centrifugation. CD4+ T cells and CD8+ T cells were future isolated and enriched using the Stemcell cell separation kits. PBMCs, CD4+ T cells and CD8+ T cells were re-suspended in cell culture medium respectively and examined for cell density and viability. Cell culture medium was used to adjust cell density to 2×10⁶ live cells/mL before 50 μL per well of cell suspension was added to flat-bottom 96-well plates. Effector cell to target cell ratio (E/T) was 20:1 for the experiments. Cell culture medium: RPMI1640 supplemented with 10% HI-FBS plus 10% Penicillin/Streptavidin.

Preparation of target cells: Raji cells were passed at 2×10⁵ cells/mL and used for experiments 4 days after passage. Proper amount of cell suspension was transferred to 50 ml tubes and centrifuged for 5 minutes at 200 g, room temperature. For flow cytometry, cells were stained by 1 μM CFSE for 20 minutes in darkness before being washed twice in PBS+5% HI-FBS. Cells were then re-suspended with cell culture medium and examined for cell density and cell viability. Cell culture medium was used to adjust cell density to 3×10⁵ live cells/mL before 50 μL per well of cell suspension was added to flat-bottom 96-well plates.

Preparation of antibodies: stocks for sample antibody #63—CD3×CD19 bispecific, Blinatumomab, MGD011 and RG6026 were diluted in cell culture medium to various concentrations. 50 l of cell culture medium or antibody dilutions were added to designated wells to reach a final antibody concentration of 0 pM, 1 pM or 100 pM.

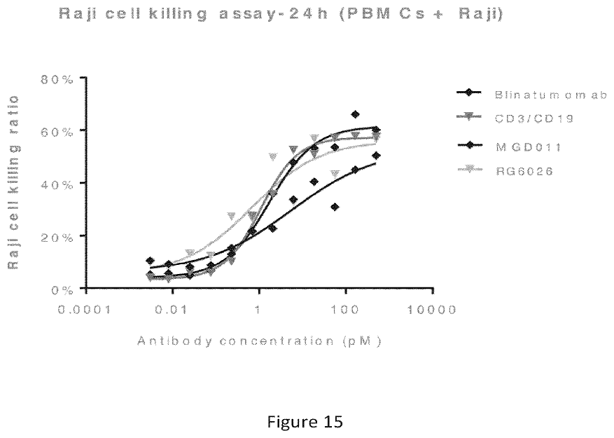

The flat-bottom 96-well plates with antibodies, target cells and effector cell (150 μL per well) were left in the incubator at 37 °C and 5% CO₂. Samples were collected at 24 hours. 10 μL of counting beads were added to each well when harvesting. The samples were centrifuged for 5 minutes at 350 g, re-suspended and stained by PI before being analyzed by flow cytometry. From results of the assays, EC50 values of sample antibody #63—CD3×CD19 bispecific, Blinatumomab, MGD011 and RG6026 were listed in Table 25. The killing effect of sample antibody #63—CD3×CD19 bispecific, Blinatumomab, MGD011 and RG6026 on target cells was shown in FIG. 15.

TABLE 25

| | Blinatumomab | CD3/CD19 | MGD011 | RG6026 |
|---|---|---|---|---|
| EC50(pM) | 5.476 | 1.086 | 1.721 | 0.6701 |

### In Vivo Effect

Antibodies were tested for their in vivo anti-tumor activity in the Jeko-1/NCG Mixeno model. On the starting day (Day 0), 5×10⁶ Jeko-1 cells suspended in 100 μL 1:1 PBS/gel was inoculated in the right flank of animals. 3 days after inoculation (Day 3) 1×10⁷/0.1 mL PBMC were injected into the abdomen of animals. Sample antibodies were administrated when average tumor size reached 100 mm³. 4 antibodies were tested (Blinatumomab@0.5 mg/kg, CD3×CD19-#63@0.3 mg/kg, MGD011@0.3 mg/kg, and RG6026@0.7 mg/kg) together with one pH6.0 PBS control group; 6 animals per group. All samples were administrated by intravenous injection into the caudal vein. All antibodies and the vehicle were administrated twice a week for 3 weeks consecutively, except for Blinatumomab which was administrated daily for 10 days. Activity was assessed based on relative tumor inhibition (TGIRTV), and safety was assessed based on animal weight change and animal death.

TGIRTV=1−TRTV/CRTV (%). Relative tumor growth inhibition rate TGIRTV (%):

TRTV/CRTV (%) is relative tumor growth rate, i.e. at a certain time point, the ratio between the tumor volume of the group that received treatment to the tumor volume of the control group that received PBS. TRTV and CRTV are tumor volume (TV) of the treatment group and the control group at a certain time point, respectively.

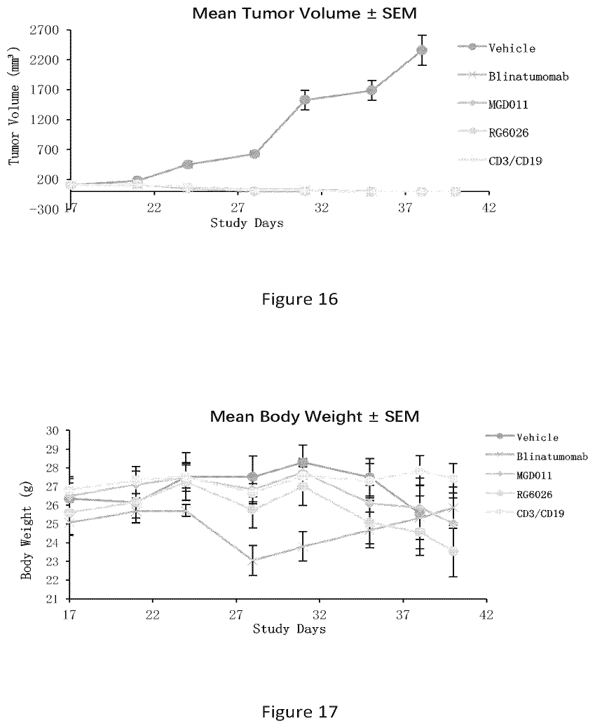

The experiment ended 39 days after inoculation. As shown in FIG. 16, all treatment (antibody) groups showed significant inhibition on tumor growth. And as shown in FIG. 17, no significant weight loss was observed in CD3×CD19 treatment group.

### Example 10

### Antibody Construction

CD3×CD19×CD8 tri-specific antibody was constructed on the basis of Example 8. Polypeptides A and B in Examples 8 were retained and polypeptides D and E were added to complete the structure. DNAs for four polypeptides (sequences for polypeptides A, B, D and E were as below) were included in vectors and transfected into CHO3E7 cells for transient expression. Expressed protein was purified using Protein A and purity of the product was over 90%. CD3×CD19×CD8 tri-specific antibody was constructed as the following.
Polypeptide A (SEQ ID NO.: 10): CD19 VL-linker-CD3 VH
Polypeptide B (SEQ ID NO.: 11): CD3 VL-linker-CD19 VH-hinge-CH2-CH3
Polypeptide D (SEQ ID NO.: 17): CD8VH-CH1-Hinge-CH2-CH3
Polypeptide E (SEQ ID NO.: 18): CD8VL-CL

Fv sequences for CD3, CD19 and CD8 were listed in sequence listing (CD19 VL: SEQ ID NO.: 13; CD19 VH: SEQ ID NO.: 14; CD3 VL: SEQ ID NO.: 15; CD3 VH: SEQ ID NO.: 16; CD8 VL: SEQ ID NO: 19; CD8 VH: SEQ ID NO.: 20). Point mutations were introduced at designated sites in FR of VH and VL domains of CD3, CD19 and CH3, as listed in the Table 26 below.

TABLE 26

| | Polypeptide | Mutation List | | | |
|---|---|---|---|---|---|
| | Chain | Domain Order | Domain 1 | Domain 2 | CH3 |
| #55 | A | CD19 VL-linker*- | Q38K, | Q39K | |
| | | CD3 VH | G100C | | |
| | B | CD3 VL-linker*- | Q38D, | Q39D | T366W |
| | | CD19 VH-hinge- | G44C | | |
| | | CH2—CH3 | | | |
| | D | CD8VH-CH1- | | | Y407V, |
| | | Hinge-CH2—CH3 | | | T366S, |
| | | | | | L368A |
| | E | CD8VL-CL | | | |

*Linker: RTVAA

### Example 11

### Affinity

Binding affinity for human CD3ε or human CD19 of the CD3×CD19×CD8 tri-specific antibody was tested by BIAcore. Kₐ, K_{d} and K_{D} were calculated from the experimental results and used to evaluate affinity of the antibodies for human CD3ε and CD19. CD3ε and CD19 molecules as ligands were immobilized to chips conjugated with anti-histine antibodies. Antibody molecules in five different concentrations were analyzed for their affinity for CD19. Affinity for human CD3c was examined using the same method. Results are listed in Table 27 below.

TABLE 27

| Target | Ka | Kd | K_{D} |
|---|---|---|---|
| CD3 | 1.41E+5 | 0.004411 | 3.13E−8 |
| CD19 | 1.15E+5 | 9.93E−4 | 8.63E−9 |

### Cell Killing Assays

Antibody-mediated killing effect on target cells (Raji cells) was assayed using lymphocytes as effector cells. Protocol as described below:

Preparation of effector cells: PBMCs were freshly isolated from human blood by density gradient centrifugation. CD4+ T cells and CD8+ T cells were further isolated and enriched using the Stemcell cell separation kits. PBMCs, CD4+ T cells and CD8+ T cells were re-suspended in cell culture medium respectively and examined for cell density and viability. Cell culture medium was used to adjust cell density to 2×10⁶ live cells/mL before 50 μL per well of cell suspension was added to flat-bottom 96-well plates. Effector cell to target cell ratio (E/T) was 20:1 for the experiments. Cell culture medium: RPMI1640 supplemented with 10% H1-FBS plus 1% Penicillin/Streptavidin.

Preparation of target cells: Raji cells were passed at 2×10⁵ cells/mL and used for experiments 4 days after passage. Proper amount of cell suspension was transferred to 50 ml tubes and centrifuged for 5 minutes at 200 g, room temperature. For flow cytometry, cells were stained by 1 pM CFSE for 20 minutes in darkness before being washed twice in PBS+5% HI-FBS. Cells were re-suspended with cell culture medium and examined for cell density and cell survival rate. Cell culture medium was used to adjust cell density to 3×10⁵ live cells/mL before 50 μL per well of cell suspension was added to flat-bottom 96-well plates with Raji cells already in.

Preparation of antibodies: stocks for sample antibody #55, Blinatumomab, MGD011 and RG6025 were diluted in cell culture medium to various concentrations. 50 μl of cell culture medium or antibody dilutions were added to designated wells to reach a final antibody concentration of 0 pM, 1 pM or 100 pM.

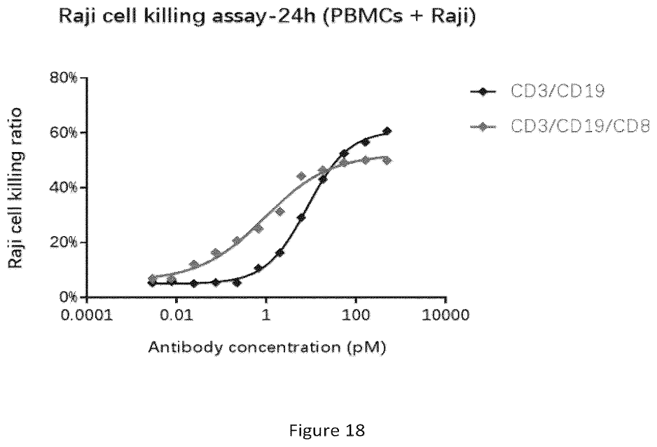

Flat-bottom 96-well plates with antibodies, target cells and effector cell (150 μL per well) were left in the incubator at 37 °C, 5% CO₂ for 24 hours. Samples were collected at 24 hours. 10 μL of counting beads were added to each well when harvesting. The samples were centrifuged for 5 minutes at 350 g, re-suspended and stained by PI before being analyzed by flow cytometry. Results of the assays revealed that EC50 of #55 was 0.948 pM. The killing effect of sample #55 on target cells was shown in FIG. 18.

### In Vivo Effect

Antibodies were tested for their in vivo anti-tumor effect in the Jeko-1/NCG Mixeno model. On the starting day (Day 0), 5×10⁶ Jeko-1 cells suspended in 100 μL 1:1 PBS/gel was inoculated in the right flank of animals. 3 days after inoculation (Day 3) 1×10⁷/0.1 mL PBMC were injected into the abdomen of animals. Sample antibodies were administrated when average tumor size reached 100 mm³. 2 antibodies at various dosages were tested (CD3×CD19@0.5 mg/kg, #55@0.1 mg/kg, #55@0.5 mg/kg, and #55@3 mg/kg) together with one pH6.0 PBS control group; 6 animals per group. All samples were administrated by intravenous injection into the caudal vein. All antibodies and the vehicle were administrated twice a week for 3 weeks consecutively. Effect was evaluated based on relative tumor inhibition (TGIRTV), and safety was evaluated on animal weight change and animal death.

TGIRTV=1−TRTV/CRTV (%). Relative tumor growth inhibition rate TGIRTV (%):

TRTV/CRTV (%) is relative tumor growth rate, i.e. at a certain time point, the ratio between the tumor volume of the group that received treatment to the tumor volume of the control group that received PBS. TRTV and CRTV are tumor volume (TV) of the treatment group and the control group at a certain time point, respectively.

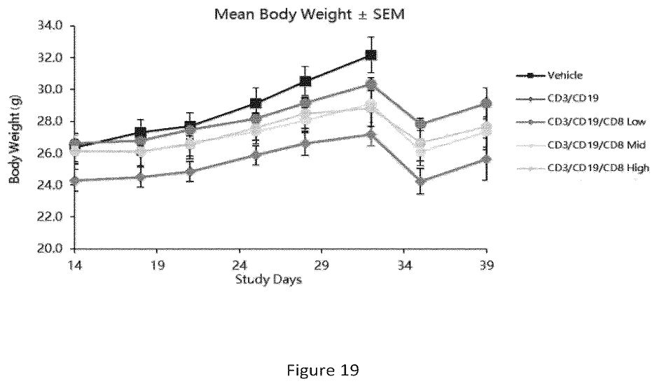

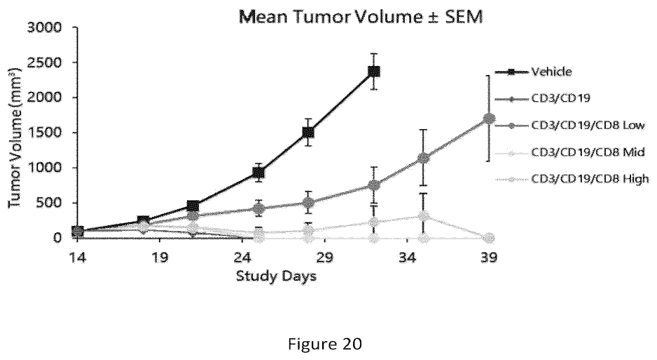

The experiment ended 39 days after inoculation. From FIG. 20, all treatment (antibody) groups showed significant inhibition on tumor growth. And as shown in FIG. 19, no significant weight loss was observed in all treatment (antibody) groups.

What is claimed is:

1. A method of manufacturing an engineered antibody comprising the steps of co-transfecting four constructs that encode the engineered antibody into a cell system, expressing the engineered antibody in a cell culture system, and
purifying the engineered antibody,
wherein the engineered antibody has a purity of over 95%,
wherein each of the four constructs encode,
(i) a first polypeptide comprising a second light chain variable domain (VL2) and a first heavy chain variable domain (VH1), wherein said VL2 is linked to said VH1;
(ii) a second polypeptide comprising a first light chain variable domain (VL1), a second heavy chain variable domain (VH2), a first hinge domain, and a first CH2-CH3 domain of IgG, wherein said VL1 is linked to said VH2;
(iii) a third polypeptide comprising a third heavy chain variable domain (VH3), a CH1 domain, a second hinge domain, and a second CH2-CH3 domain of IgG; and
(iv) a fourth polypeptide comprising a third light chain variable domain (VL3) and a CL domain; respectively,
wherein the VL2 and VH2 are covalently linked via a disulfide bond; and
wherein the VH2 and VL2 are associated via electrostatic interaction formed between a pair of oppositely charged amino acid residues in VH2 and VL2, respectively.

2. The method of claim 1, wherein the disulfide bond is formed between cysteine substitutions at position 100 of the VL2 and position 44 of the VH2, or alternatively at position 43 of the VL2 and position 105 of the VH2 according to Kabat EU index.

3. The method of claim 2, wherein the pair of oppositely charged amino acid residues is at position 38 of the VL2 and position 39 of the VH2, or alternatively at position 44 of the VL2 and position 103 of the VH2 according to Kabat EU index.

4. The method of claim 3, wherein the pair of oppositely charged amino acid residues comprise a negatively charged amino acid residue selected from aspartic acid (D) or glutamic acid (E), and a positively charged amino acid residue selected from lysine (K), histidine (H) or arginine (R).

5. The method of claim 3, wherein the pair of oppositely charged amino acid residues comprise:
a) 38D in the VL2 and 39K in the VH2, respectively;
b) 38K in the VL2 and 39D in the VH2, respectively,
c) 44K in the VL2 and 103D in the VH2, respectively; or
d) 44D in the VL2 and 103K in the VH2, respectively;
wherein numbering is according to the Kabat index.

6. The method of claim 1, wherein:
a) the VL2 comprises 100C and 38D, and VH2 comprises 44C and 39K;
b) the VL2 comprises 100C and 38K, and VH2 comprises 44C and 39D;
c) the VL2 comprises 43C and 38D, and VH2 comprises 105C and 39K;
d) the VL2 comprises 43C and 38K, and VH2 comprises 105C and 39D;
e) the VL2 comprises 100C and 44K, and VH2 comprises 44C and 103D;
f) the VL2 comprises 100C and 44D, and VH2 comprises 44C and 103K;
g) the VL2 comprises 43C and 44K, and VH2 comprises 105C and 103D; or
h) the VL2 comprises 43C and 44D, and VH2 comprises 105C and 103K.

7. The method of claim 1, wherein the VL2 and VH1 domains are linked via a first peptide linker and wherein the VL1 and VH2 domains are linked via a second peptide linker.

8. The method of claim 7, wherein the first and second peptide linker each comprises 5-9 amino acids.

9. The method of claim 1, wherein the first CH2-CH3 region and the second CH2-CH3 region are different.

10. The method of claim 9, wherein
(a) one of the CH3 domains comprises a replacement of Thr366 with Trp, and the other CH3 domain comprises a replacement of Thr366, Leu368, Tyr407 with Ser, Ala and Val, respectively;
(b) one of the CH3 domains comprises a replacement of Asp399 and Glu356 with Lys, and the other CH3 domain comprises a replacement of Lys392 and Lys409 with Asp;
(c) one of the CH3 domains comprises a replacement of Glu356, Glu357 and Asp399 with Lys, and the other CH3 domain comprises a replacement of Lys370, Lys409 and Lys439 with Glu, Asp and Glu, respectively;
(d) one of the CH3 domains comprises a replacement of Ser364 and Phe405 with His and Ala respectively, and the other CH3 domain comprises a replacement of Tyr349 and Thr394 with Thr and Phe, respectively; or
(e) one of the CH3 domains comprises a replacement of Lys370 and Lys409 with Asp, and the other CH3 domain comprises a replacement of Glu357 and Asp399 with Lys; or
(f) one of the CH3 domains comprises a replacement of Leu351 and Leu368 with Asp and Glu respectively, and the other CH3 domain comprises a replacement of Leu361 and Thr366 with Lys.

11. The method of claim 1, wherein the engineered antibody is a multi-specific antibody.

12. The method of claim 1, wherein the VL1 and VH1 domain, the VL2 and VH2 domain, and the VL3 and VH3 domain target a first target, a second target and a third target, respectively.

13. The method of claim 12, wherein the first target and the second target, or the first target and the third target, or the second target and the third target, recognize the same target.

14. The method of claim 12, wherein the first, second and third target each is different.

15. The method of claim 13, wherein one of the three targets is CD3.

16. The method of claim 15, wherein one of the three targets is CD19.

17. The method of claim 1, wherein the engineered antibody has a purity of over 96%.

18. The method of claim 1, wherein the engineered antibody has a purity of over 99%.

19. The method of claim 1, wherein the cell system comprises stable cells that are transfected to express the engineered antibody.

20. The method of claim 19, wherein the stable cells are mammalian cells.

21. An engineered antibody that is produced by the method of claim 1.

22. The engineered antibody of claim 21, wherein the engineered antibody is stable for at least 10 days at 4 °C or 25 °C.

23. The engineered antibody of claim 22, wherein the antibody has a purity of over 95%.
